(19) United States
(12) Patent Application Publication
(10) Pub. No.: US 2026/0170410 A1
(43) Pub. Date: Jun. 18, 2026

(54) CLOSED-LOOP OPTIMIZATION OF GENERAL REACTION CONDITIONS FOR HETEROARYL SUZUKI-MIYUARA COUPLING

(71) Applicants: The Board of Trustees of the University of Illinois, Urbana, IL (US); Allchemy, Inc., Highland, IN (US)

(72) Inventors: Martin D. Burke, Champaign, IL (US); Nicholas H. Angello, Champaign, IL (US); Vandana Rathore, Urbana, IL (US); Wiktor Beker, Wroclaw (PL); Agnieszka Wolos, Gdynia (PL); Rafal Roszak, Wroclaw (PL); Sara Szymkuc, Warsaw (PL)

(21) Appl. No.: 19/124,566

(22) PCT Filed: Oct. 25, 2023
(86) PCT No.: PCT/US23/35923

§ 371 Date: Apr. 25, 2025

Related U.S. Application Data
(60) Provisional application No. 63/419,702, filed on Oct. 26, 2022.

(51) Int. Cl. G06N 20/00; C07D 401/12; G06Q 10/04

(52) U.S. Cl. CPC G06N 20/00; C07D 401/12; G06Q 10/04

(57) ABSTRACT

Disclosed are systems and methods for rapidly generating general reaction conditions using a closed-loop workflow leveraging matrix down-selection, machine learning, and robotic experimentation.

### RELATED APPLICATIONS

This application claims the benefit of priority to U.S. Provisional Application No. 63/419,702, filed Oct. 26, 2022.

### STATEMENT OF GOVERNMENT SUPPORT

This invention was made with government support under HR00111920027 awarded by the U.S. Defense Advance Research Projects Agency (DARPA). The government has certain rights in the invention.

### BACKGROUND

General conditions for organic reactions are important but rare, and efforts to find them usually consider only narrow regions of chemical space. Discovering more general reaction conditions requires considering vast regions of chemical space derived from a large matrix of substrates crossed with a high-dimensional matrix of reaction conditions, rendering exhaustive experimentation impractical.

Embodiments of the present disclosure use a simple closed-loop workflow that leverages data-guided matrix down-selection, uncertainty-minimizing machine learning, and robotic experimentation to discover general reaction conditions. Application to the challenging and important problem of heteroaryl Suzuki-Miyaura cross-coupling identified conditions that double the average yield relative to a widely used benchmark that was previously developed using traditional approaches. This disclosure provides a practical roadmap for solving multidimensional chemical optimization problems with large search spaces.

### SUMMARY OF THE INVENTION

In certain aspects, provided is a method, comprising:
selecting a reaction pair comprising a first molecule and a second molecule;
wherein the first molecule is selected from a first matrix and the second molecule is selected from a second matrix;
selecting one or more conditions for a reaction of the selected reaction pair, the selection based on historic use of the condition and a structural and functional diversity of the selected reaction pair;
automatically performing, by a robotic system, an initial round of reactions between the selected reaction pair under the selected one or more conditions;
optimizing, using a machine learning model, one or more reaction conditions associated with the initial round of reactions based on a yield of each reaction in the initial round of reactions;
determining an optimized series of reactions to be performed;
performing a reaction of the optimized series of reactions, thereby forming a product, and predicting yields thereof; and
outputting an optimized set of general reaction conditions for each optimized reaction between the selected reaction pair.

In certain aspects, provided is a system, comprising:
a robotic system; and
   a computing node comprising a computer readable storage medium having program instructions embodied therewith, the program instructions executable by a processor of the computing node to cause the processor to perform a method comprising the method described herein.

### BRIEF DESCRIPTION OF THE DRAWINGS

FIG. 1 is a flowchart of an exemplary process for generating general reaction conditions, according to techniques presented herein.

FIG. 2 depicts a general workflow for the discovery of general reaction conditions.

FIG. 3A depicts T-distributed stochastic neighbor embedding (t-SNE) mapping of substrate combinations.

FIG. 3B illustrates t-SNE mapping of the product space synthesized during the training and test sets compared to the overall reaction space.

FIG. 3C illustrates a reaction scheme and chemical structures of an initial training set.

FIG. 3D illustrates a robotic system for the automatic performance of all reactions.

FIG. 3E is a table illustrating all couplings performed between pairs of substrates, each under conditions corresponding to the JACS 2009 benchmark.

FIG. 3F is a graphic illustrating the Spearman rank matrix.

FIG. 4A illustrates convergence of the model's uncertainty.

FIG. 4B illustrates a comparison of ML-guided vs. random searches for general conditions.

FIG. 4C illustrates a comparison of yield distribution between literature-reported reactions and those performed in the present disclosure.

FIG. 4D illustrates the model gaining the ability to accurately categorize these conditions into high, medium, and low overall average yield, and, in the subsequent rounds, establishes the correct ranking within these categories.

FIG. 4E illustrates the ranking per general condition per round is illustrated as perceived by the ML model.

FIG. 4F shows the model's ranking uncertainty decreasing between rounds and is especially apparent for the top conditions.

FIG. 4G illustrates the model's choice to test a few substrates per round across many conditions for the first 3 rounds, followed by primarily filling in the top conditions in the latter rounds.

FIG. 4H shows that by the fifth round, the model explored nearly all of the top 7 conditions, which corresponds to every condition with >50% overall average yield as estimated by the model.

FIG. 4I shows the yields of reactions the model requested analyzed in order to gain more information about the reaction-condition space.

FIG. 5A shows a set of 20 diverse compounds from outside of the training set selected to test whether the discovered general reaction conditions translate to other diverse heteroaryl product classes. JACS 2009:5:1 Dioxane:water, 60° C., K₃PO₄, Pd SPhos G4. ML General condition 1:5:1 Dioxane:water, 100° C., Na₂CO₃, Pd XPhos G4. ML General condition 2:5:1 Dioxane:water, 100° C., Na₂CO₃, Pd SPhos G4. ML General condition 3:5:1 dioxane:water, 100° C., Na₂CO₃, Pd(PPh₃)₄.

FIG. 5B shows a jitter plot of the performance of the top ML conditions versus the benchmark. Brackets indicate 95% confidence interval.

FIG. 5C shows a jitter plot of the relative performance in change of yield of the top ML conditions versus the benchmark. Brackets indicate 95% confidence interval.

FIG. 5D shows the number of products per general condition with >10% yield measured.

FIG. 5E shows the relative protodeboronation per condition as measured by integrated UV peak area (UV_{PDB}) standardized to the internal standard (UV_{STD}).

FIG. 5F shows the relative remaining halide per condition as measured by integrated UV peak area (UV_{HAL}) standardized to the internal standard (UV_{STD}).

FIG. 5G Relative product formation per condition (UV_{PDT}) relative to byproduct formation (UV_{BYPDT}).

FIG. 6 depicts an exemplary computing node.

FIG. 7 depicts a schematic of the automated synthesis machine.

FIG. 8 depicts a photograph of the automated synthesis machine

FIG. 9 depicts valve tubing connection diagram. 1-A to 5-D refer to reaction vial connections from 1-36 sequentially. S1 to S20 refer to solvent reservoirs.

FIG. 10 depicts a schematic of the Argon/vacuum manifold. Sole=solenoid valve. Opening and closing solenoid valves connected to Argon and a vacuum pump allowed for an automated Schlenk line vacuum/backfill process.

FIG. 11 depicts 4 Channel Solenoid Driver schematic.

FIG. 12 depicts top: Circular heating block design, units are in inches. Bottom: In-situ measured reaction temperature per vial between two 12-reaction vial heating block configurations. Each vial was filled with 8 mL of dimethylsulfoxide (DMSO) and the hotplate was turned on and set to 85° C. and allowed to equilibrate (˜30 min). Temperature was measured by submerging a thermometer in the heated DMSO solution of each vial until it read a constant temperature (˜1 min.). “X” denotes heating probe placement.

FIG. 13 depicts Lab VIEW code ‘front panel.’ Code is started by clicking the arrow in the top left.

FIG. 14 depicts automated test reactions probing boron speciation. Experiments were performed in 8 parallel replicates. Data represented in a box and whisker plot showing maximum and minimum values observed. Solid components were weighed into reaction vials in an argon filled glovebox (due to air sensitivity of the Pd catalyst) and sealed under argon prior to loading onto the automated synthesizer. Reactions were conducted on 0.1 mmol scale of halide with 3 equiv. of boron species, 1 equiv. of phenanthrene internal standard, 7.5 equiv. of Na₂CO₃, and 5 mol % of Pd(PPh₃)₄ catalyst in 8 mL of argon-sparged 5:1 dioxane:water solvent.

FIG. 15 depicts manual test reactions probing inert environment. Reactions were conducted on 0.1 mmol scale of halide with 3 equiv. of boron species, 1 equiv of phenanthrene internal standard, 7.5 equiv. of K₃PO₄, and 5 mol % of Pd SPhos G4 catalyst in 8 mL of 5:1 dioxane:water solvent.

FIG. 16 depicts performance of the Automated Schlenk line. An Auto Schlenk cycle consisted of 5-minute vacuum followed by 1-minute argon. Reactions were conducted on 0.1 mmol scale of halide with 3 equiv of boron species, 1 equiv of phenanthrene internal standard, 7.5 equiv of K₃PO₄, and 5 mol % of Pd SPhos G4 catalyst in 8 mL of 5:1 dioxane:water solvent. Reactions were conducted in parallel by alternating between introducing a reaction vial onto the instrument and running a Schlenk cycle.

FIG. 17 depicts schematic architecture of neural components used in GP models.

FIG. 18A depicts dependence of models' (NNE and GPE) prediction errors and uncertainties on the relative size of the dataset used for training. For each size of the training set, the training data were randomly selected from the whole Santanilla (16) dataset. The remaining portion of the dataset was used as a test set. The dataset selection and model training were repeated 60 times, and the average values are depicted as points and lines, whereas standard deviations from these averages are denoted by shaded areas. We assign orange color to GPE and blue to NNE. A) Mean absolute error (MAE), solid lines=test set, dotted lines=training set.

FIG. 18B depicts Kendall t measuring rank correlation between model's absolute errors and uncertainties.

FIG. 18C depicts model's uncertainty (solid lines=test set, dotted lines=training set).

FIG. 18D depicts z-score: absolute error divided by the model's uncertainty.

FIG. 19 depicts selection of the next reaction.

FIG. 20 depicts comparison of GP(NN), NNE and GPE(NN) using different acquisition functions and substrate selection strategies. Vertical axis represents actual/experimental yield for conditions predicted by a given model to be the best.

FIG. 21 depicts comparison of GP(NN), NNE and GPE(NN) using different acquisition functions and substrate selection strategies. Rank represents the position at which the actual best conditions are classified by a given method (see text for details)

FIG. 22 depicts probability that a GPE(NN) model of a given size will make the same choice as the reference GPE(NN) with 2000 models. For each acquisition function (EI and PI), two probabilities are considered: probability of selecting the same conditions as the reference model (EI_cond and PI_cond) and probability of simultaneously selecting the same conditions and substrates (PI_maxUnc and EI_maxUnc).

FIG. 23 depicts comparison of GPE(NN) with different number of models and different substrate selection schemes. Rank represents the position at which actual/experimental best conditions are classified by a given method (see text for details).

FIG. 24 depicts comparison of GPE(NN) against different models and different substrate selection schemes. Vertical axis represents actual/experimental yield for conditions predicted by a given model as being the best.

FIG. 25A depicts Simulations of the information gap, σ̅-σ̅ₜᵣₐᵢₙ·(A) Evolution of the information gap as the simulation of the closed-loop experiments proceeds. Solid line=mean value from 100 replicas, shaded area=region within one standard deviation from the mean value.

FIG. 25B depicts information gap as the stop criterion. For each value of the threshold (X axis), simulation replicas were stopped as soon as they reached the threshold. Next, the results were used to compute the probability that true general conditions are within top-3 (orange) and top-5 (blue) predictions. The shaded areas denote the region within one standard deviation (computed as a square root of variance from the corresponding Beta distribution) from the estimate (solid lines).

FIG. 26 depicts stratified clusterization strategy for selecting representative halide building blocks.

FIG. 27 depicts 54 diverse halide centroids representing 5354 purchasable halides.

FIG. 28 depicts 54 (hetero)aryl MIDA boronates selected and purchased.

FIG. 29 depicts heteroaryl substrate scope.

FIG. 30 depicts T-distributed stochastic neighbor embedding (t-SNE) mapping of the product space explored in reference (14) (‘JACS 2009’) and this work. Blue circles, products belonging to the reported search space; green triangles, products belonging to the training set; yellow stars, products belonging to the test set. Red x marks, products belonging to the substrate scope of JACS 2009.

FIG. 31 depicts a citation report for JACS 2009, showing sustained and perhaps increased relevance of this publication over time.

FIG. 32 depicts time course of reaction condition testing per product during closed-loop experiments. Each row of the matrix corresponds to one of the 11 products in the training set, whereas columns describe the reaction conditions in the considered reaction space, sorted according to the average yield computed after all rounds (with blanks filled with the final model's predictions). The color of each tile represents the round in which the experiment was suggested by the ML model and measured, where black indicates data that was not measured in any round.

FIG. 33 depicts evolution of the average experimental yield of the predicted top-k (k=1,2,3) conditions during closed-loop experiments.

FIG. 34A depicts comparison of the measured yield of the training set under the JACS 2009 benchmark general reaction condition (mean=63.9%) vs the top ML-discovered general reaction condition at the 5^{th} round of closed-loop optimization (mean=72.4%). Brackets indicate 95% confidence interval.

FIG. 34B depicts percent change in yield relative to the benchmark condition when employing the top ML-discovered general reaction condition at the 5^{th} round of closed-loop optimization (mean=58.9% but this number is inflated due to the pair of substrates for which the yield increased >500%). Bracket indicates 95% confidence interval.

FIG. 35A-FIG. 35D depicts simulations of a protocol aiming to find general conditions by optimization of individual reactions. The experiments were performed on two datasets: our calibration dataset (Buchwald-Hartwig, BH, reactions from (16) and the hetSMC data collected in our experiments (with missing values filled with last model's predictions; panels C and D). Results for each dataset are quantified by two heatmaps: one describing the top-1 statistics depending on the values of N_{subs} and N_{wait} (panels A and C) and the other showing the corresponding maximum number of experiments ‘performed’ in these simulations.

FIG. 36 depicts example LCMS trace. TIC=total ion count (all MS signal), TWC=total wavelength count (sum of UV signals), EIC=extracted ion count (MS signal corresponding to selected ion).

FIG. 37 depicts an example MS spectrum. Full mass spectrum associated with the EIC peak of FIG. 36.

FIG. 38 depicts % yield (y-axis) versus product: internal standard ratios for Suzuki-Miyaura cross coupling product 1. Product: internal standard ratios for cross coupling products 2-30 were determined similarly.

FIG. 39A-FIG. 39E depict time course reactions for test set molecules under the Benchmark general condition.

FIG. 40 depicts chromatograms of byproduct distributions for test set reaction 12. Byproduct distributions for remaining test set reactions were determined similarly.

FIG. 41 depicts relative protodehalogenation per condition as measured by integrated UV peak area (UV_{PDH}) standardized to the internal standard (UV_{STD}).

FIG. 42 depicts P value as a function of sample size. P-values of main text FIG. 4B (comparing percent yield) calculated as a function of sample size by pMoSS: p-value Model using the Sample Size. This method models the Mann-Whitney U-test p-value as a sample size dependent function using Monte Carlo cross-validation.

### DETAILED DESCRIPTION OF THE INVENTION

In certain aspects, provided is a method, comprising:
selecting a reaction pair comprising a first molecule and a second molecule;
wherein the first molecule is selected from a first matrix and the second molecule is selected from a second matrix;
selecting one or more reaction conditions for the reaction pair, the selection based on historic use of the one or more reaction conditions and a structural and functional diversity of the selected reaction pair;
automatically performing, by a robotic system, an initial round of reactions between the selected reaction pair under the selected one or more reaction conditions;
optimizing, using a machine learning model, the one or more reaction conditions associated with the initial round of reactions based on a yield of each reaction in the initial round of reactions;
determining an optimized series of reactions to be performed;
performing a reaction of the optimized series of reactions, thereby forming a product, and predicting yields thereof; and
outputting an optimized set of general reaction conditions for each optimized reaction between the selected reaction pair.

In certain embodiments, the method further comprises minimizing uncertainty in the machine learning model.

In further embodiments, minimizing uncertainty comprises:
constructing a surrogate model to predict one or more reaction yields from the reaction pair;
estimating an objective function of a performed reaction based on a predicted output from the surrogate model; and
estimating the objective function for an unperformed reaction.

In certain embodiments, selecting a reaction pair comprises:
clustering the first matrix by a common ring structure and pendant functionality, thereby generating a first centroid; and wherein the first centroid comprises the closest representatives of the first matrix;
selecting the second molecule from the second matrix;
identifying all combinations comprising the first centroid and the second molecule, thereby generating a chemical space; and
comparing the chemical space to a corpus of chemical products, thereby generating a product space.

In further embodiments, the method further comprises:
applying a greedy algorithm to the product space; and
identifying a set of first centroid and second molecule pairs to maximize mutual dissimilarity of a resulting product of the reaction pair.

In certain embodiments, selecting the one or more reaction conditions further comprises:
considering at least one of a solvent, a base, a catalyst, and a temperature as a variable for the one or more reaction conditions; and
determining, based on analysis of prior use of the one or more reaction conditions, a set of initial conditions.

In further embodiments, the solvent is 5:1 dioxane: water.

In yet further embodiments, the base is K₃PO₄ or Na₂CO₃.

In still further embodiments, the catalyst is a palladium catalyst, and optionally further comprises a ligand.

In certain embodiments, the ligand is selected from the group consisting of SPhos, XPhos, and triphenylphosphine (PPh₃).

In further embodiments, the catalyst is selected from the group consisting of Pd(SPhos) G4, Pd(PPh₃)₄ and Pd(XPhos) G4.

In yet further embodiments, the temperature is about 50° C. to about 150° C.

In still further embodiments, the temperature is about 60° C. or about 100° C.

In certain embodiments, optimizing the one or more reaction conditions comprises:
performing a reaction under the one or more reaction conditions, thereby forming a product; and
determining parameters for the one or more reaction conditions outputting the highest yield.

In further embodiments, optimizing the one or more reaction conditions comprises:
identifying one or more catalysts with similar yields for different substrates; and
removing the identified one or more catalysts from possible reaction conditions, thereby decreasing redundancy.

In certain embodiments, the method further comprises:
iteratively repeating the optimizing of the one or more reaction conditions, thereby forming a product;
measuring a yield of the product; and
determining a threshold yield is met.

In certain embodiments, the method further comprises:
generating, by the machine learning model, small datasets for optimization,
wherein the small datasets include negative data.

In certain embodiments, the first molecule comprises a halo-substituted aryl or heteroaryl.

In further embodiments, the first molecule comprises a compound of formula (Ia):

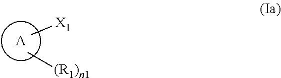

wherein:
A is aryl or heteroaryl;
each R₁ is independently selected from the group consisting of alkyl, alkoxyl, alkenyl, alkynyl, aralkyl, heteroaryl(alkyl), aryl, heteroaryl, halo, haloalkyl, hydroxyl, carboxyl, acyl, ester, amino, amido, cyano, cycloalkyl, and heterocycloalkyl;
n1 is 0, 1, 2, 3, 4, or 5; and
X₁ is halo.

In yet further embodiments, X₁ is bromo.

In certain embodiments, the first molecule is selected from the group consisting of:

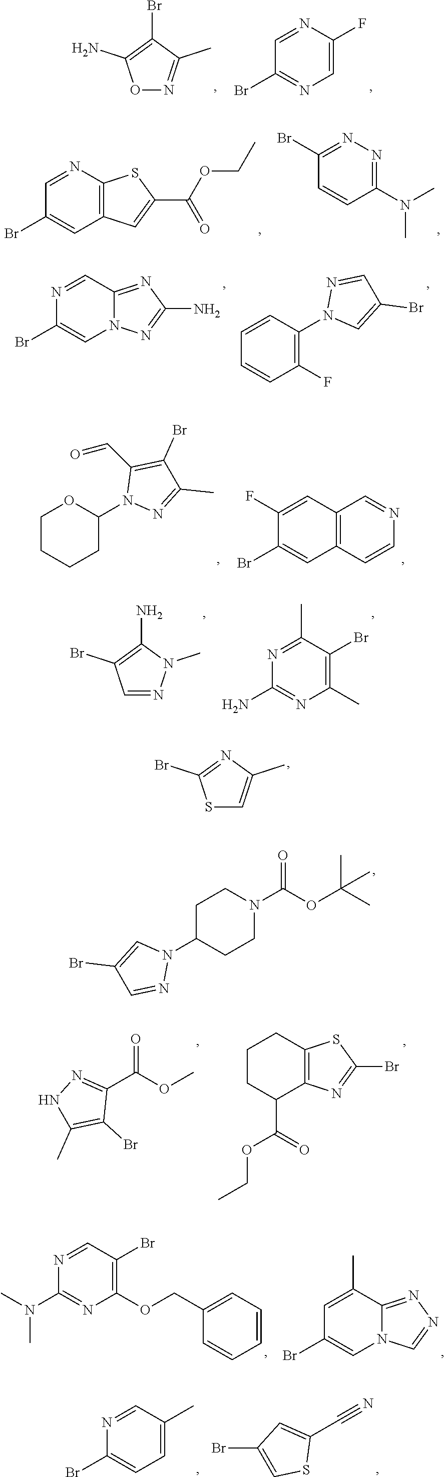

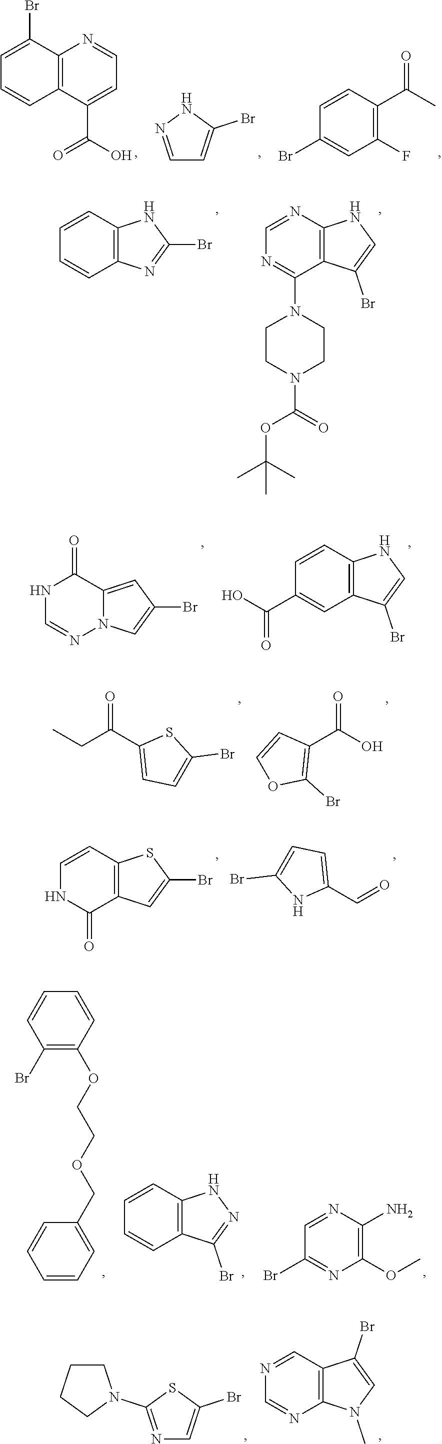

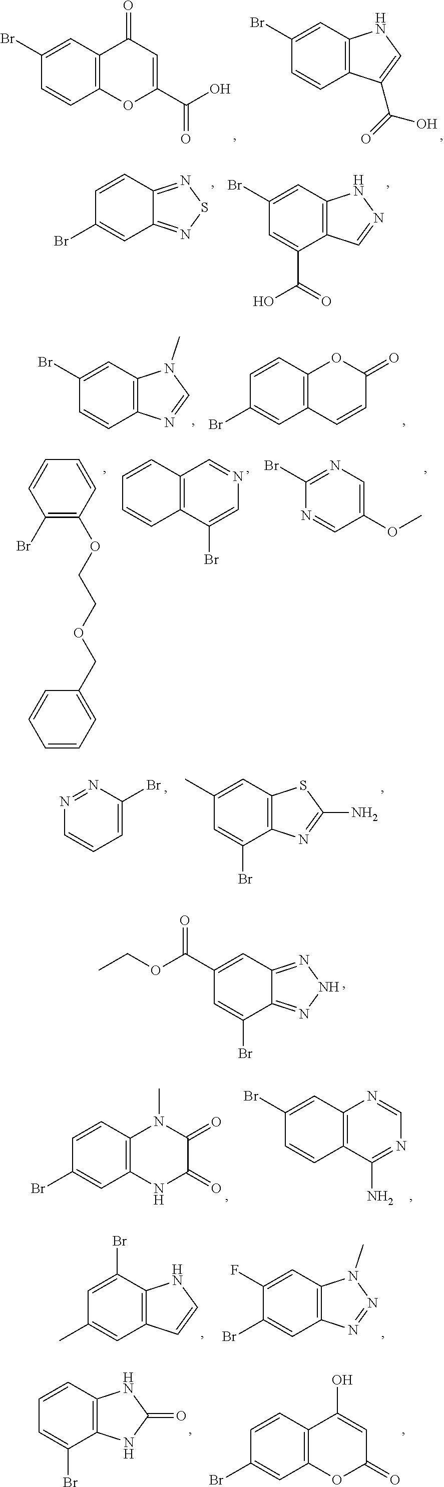

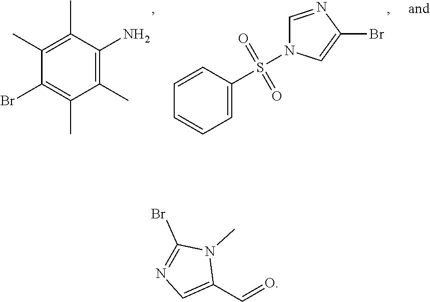

In certain embodiments, the second molecule comprises an aryl or heteroaryl further comprising a boronic acid, a boronic acid ester, or a tetrafluoroborate salt.

In further embodiments, the second molecule comprises a compound of formula (Ib):

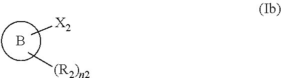

wherein:
B is aryl or heteroaryl;
each R₂ is independently selected from the group consisting of alkyl, alkoxyl, alkenyl, alkynyl, aralkyl, heteroaryl(alkyl), aryl, heteroaryl, halo, haloalkyl, hydroxyl, carboxyl, acyl, ester, amino, amido, cyano, cycloalkyl, and heterocycloalkyl;
n2 is 0, 1, 2, 3, 4, or 5; and
X₂ is selected from the group consisting of N-methylimidodiacetic acid boronic acid ester, tetramethyl N-methyliminodiacetic acid boronic acid ester, pinacol boronic acid ester, boronic acid, or a tetrafluoroborate salt.

In yet further embodiments, the second molecule is selected from the group consisting of:

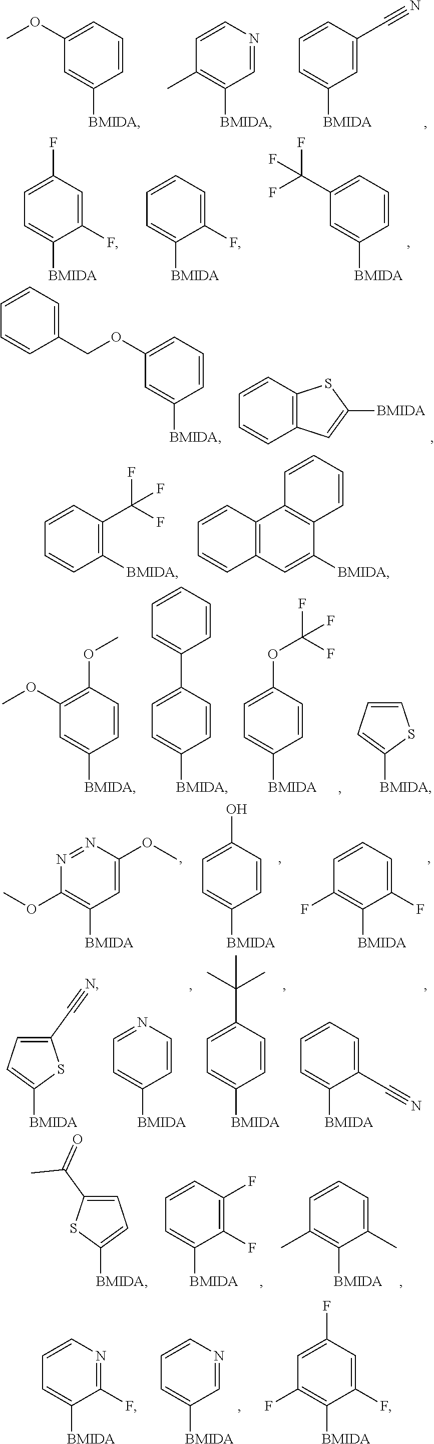

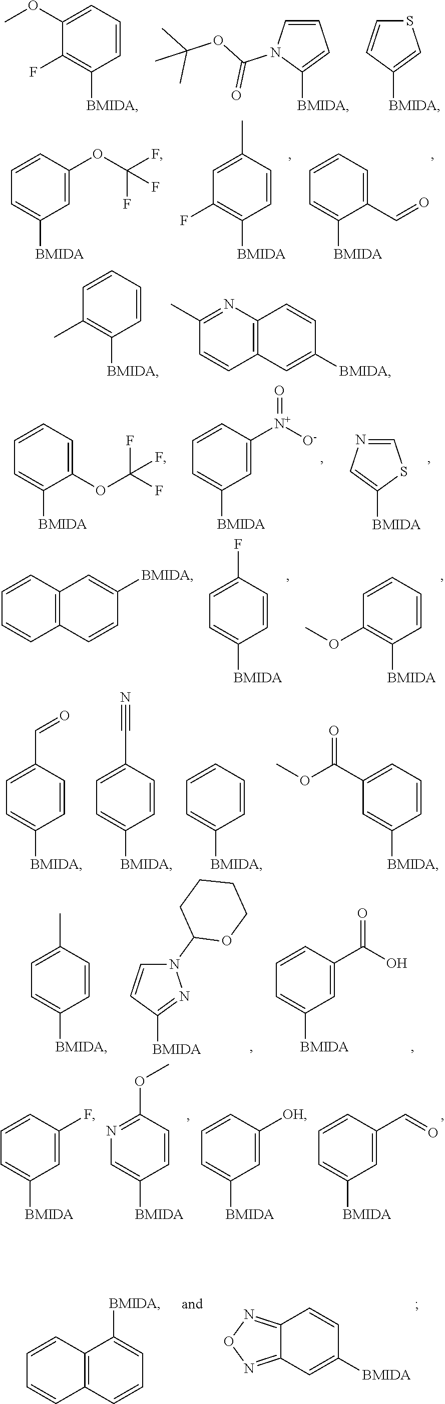

wherein BMIDA is N-methylimidodiacetic acid boronic acid ester.

In certain aspects, provided is a system, comprising:
a robotic system; and
a computing node comprising a computer readable storage medium having program instructions embodied therewith, the program instructions executable by a processor of the computing node to cause the processor to perform a method described herein.

The development of automated synthesis methods for peptides, nucleic acids, and polysaccharides requires discovery of highly general reaction conditions applicable to a wide range of building block combinations. In contrast, in the synthesis of small organic molecules, bespoke reaction conditions are usually developed to maximize the yield of each target molecule, minimize side products, and/or minimize the cost of the corresponding process. This is often necessary because synthetic methods are typically optimized on only one or a few pairs of substrates, and then applied to a wider range of substrate combinations with the rarely fulfilled hope that the same conditions will generally lead to high yields. Even the application of machine learning to optimization protocols does not ensure generality, which is critical for automating, accelerating, and ultimately democratizing the small molecule making process. Identification of such general conditions is difficult because the search space—spanning all possible combinations of substrates multiplied by all possible combinations of reaction conditions—is enormous, and impractical to navigate using standard approaches.

Heteroaryl molecular fragments are ubiquitous in many industrially relevant functional molecules, including pharmaceuticals, materials, catalysts, dyes, and natural products. In all of these spaces, synthesis remains a key bottleneck. Finding general conditions for (hetero)aryl Suzuki-Miyaura cross-coupling (SMC) is thus an important problem. It is also a challenging and largely unsolved problem, due primarily to variable degrees of both desired and undesired reactivities across the very large and diverse range of potential heteroaryl and aryl substrates. Embodiments of the present disclosure use machine learning (ML) to discover general reaction conditions by mining the extensive chemical literature on (hetero)aryl SMC.

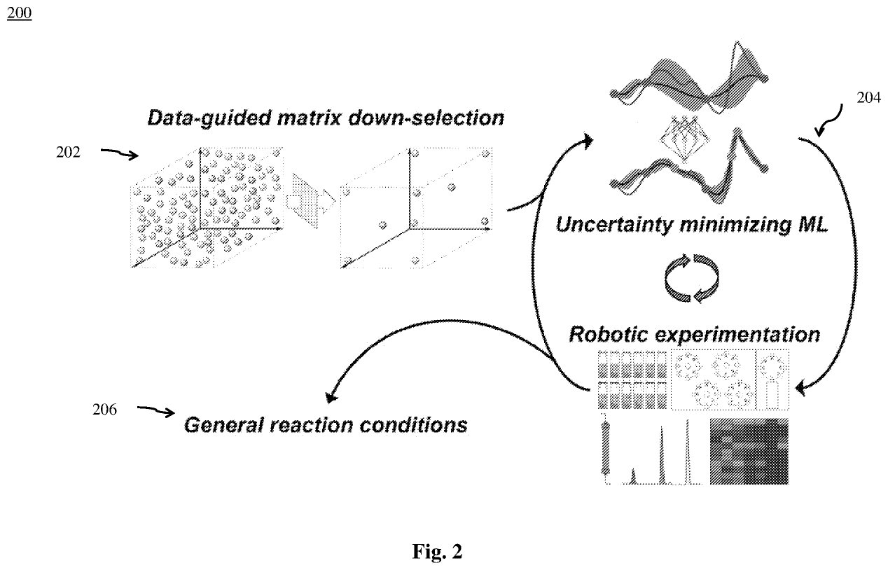

Here, a simple closed-loop workflow that can efficiently navigate vast substrate-condition space to discover general reaction conditions is reported. The approach leverages: i) data-guided matrix down-selection to render the vast search space tractable while retaining validity to the whole; ii) uncertainty-minimizing ML to efficiently drive prediction optimization; and iii) robotic experimentation to increase throughput, precision, and reproducibility of data sets recursively generated on demand (FIG. 2). This workflow succeeds in identifying general reaction conditions for the (hetero)aryl SMC reaction. The solution doubled the average yield compared to benchmark general conditions that had previously been developed through traditional human-guided experimentation, and which have since been used extensively in academic and industrial laboratories worldwide (cited in >590 papers and patent applications). This approach can thus find impactful solutions that lie in vast multidimensional search spaces, and stands to accelerate the field of organic chemistry's march towards automated and democratized small molecule synthesis, which critically requires more general reaction conditions.

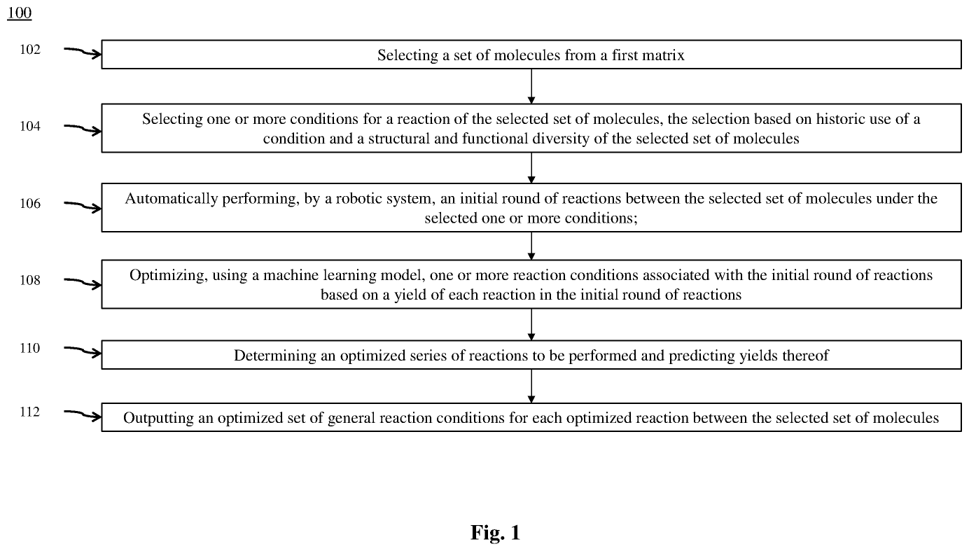

FIG. 1 is a flowchart of an exemplary process 100 for generating general reaction conditions. For example, process 100 (i.e., steps 102-112) can be automatic or initiated in response to a user input. In step 102, the process 100 may include selecting a set of molecules from a first matrix. The molecules may be strategically down-selected to define a chemical space

In step 104, the process 100 may include selecting one or more conditions for a reaction of the selected set of molecules, the selection based on historic use of a condition and a structural and functional diversity of the selected set of molecules. In step 106, the process 100 may include automatically performing, by a robotic system, an initial round of reactions between the selected set of molecules under the selected one or more conditions. The robotic system may be the automated synthesis described herein.

In step 108, the process 100 may include optimizing, using a machine learning model, one or more reaction conditions associated with the initial round of reactions based on a yield of each reaction in the initial round of reactions. Optimizing the conditions may include minimizing uncertainty within the machine learning model. In step 110, the process 100 may include determining an optimized series of reactions to be performed and predicting yields thereof. In some embodiments, the process may include minimizing uncertainty in the machine learning model.

In step 112, the process 100 may include outputting an optimized set of general reaction conditions for each optimized reaction between the selected set of molecules.

FIG. 2 illustrates an embodiment of a simple closed-loop workflow that can efficiently navigate vast substrate-condition space to discover general reaction conditions. In step 202, data-guided matrix down-selection occurs, creating a chemical space for the generation of general reaction conditions. In step 204, a closed loop is created between an uncertainty-minimizing machine learning model and a robotic experimentation. In step 206, general reaction conditions are determined and output from the iterative closed loop process.

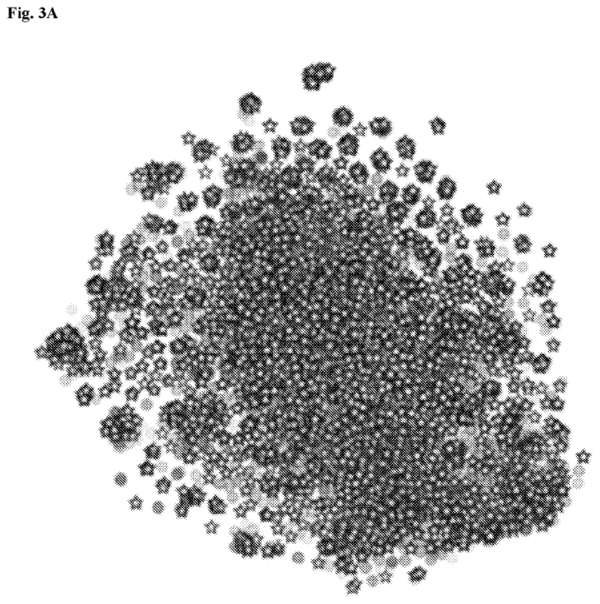

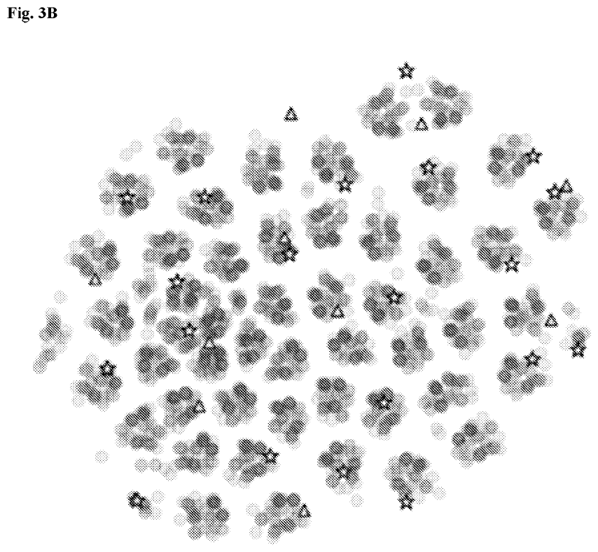

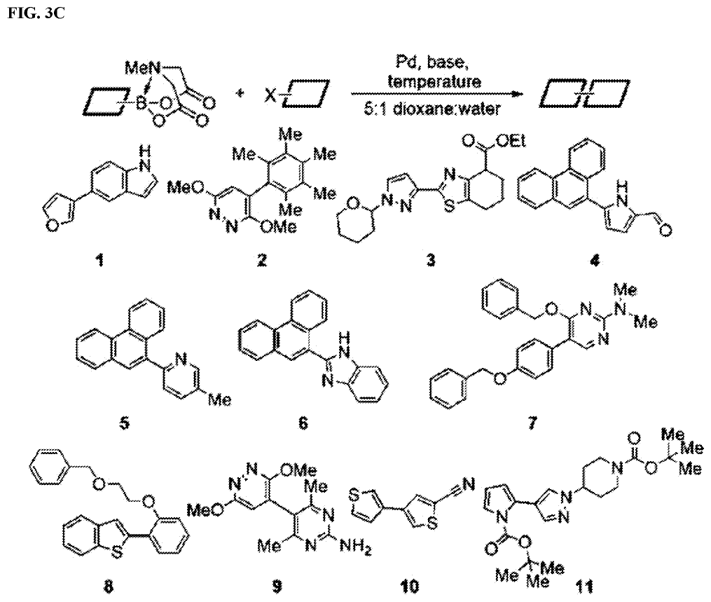

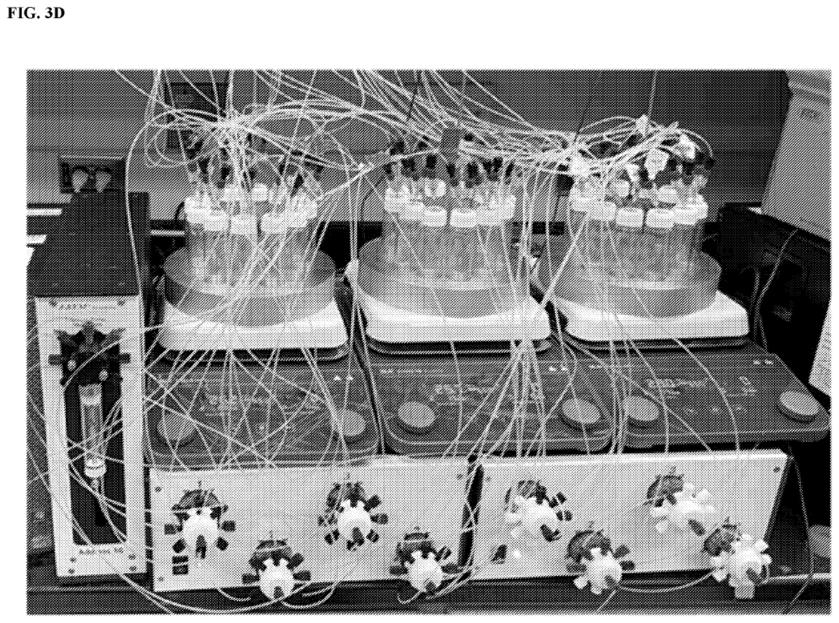

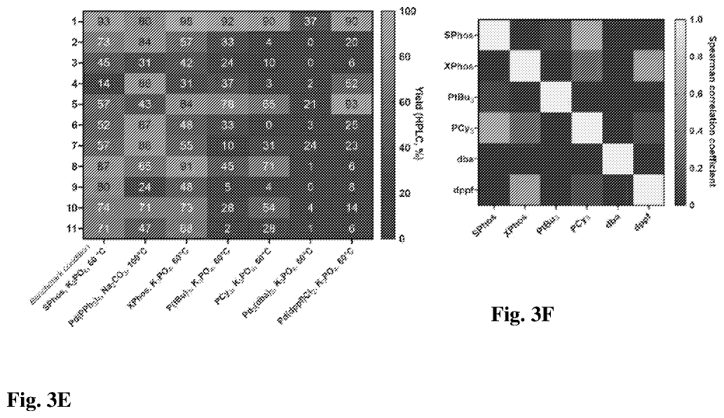

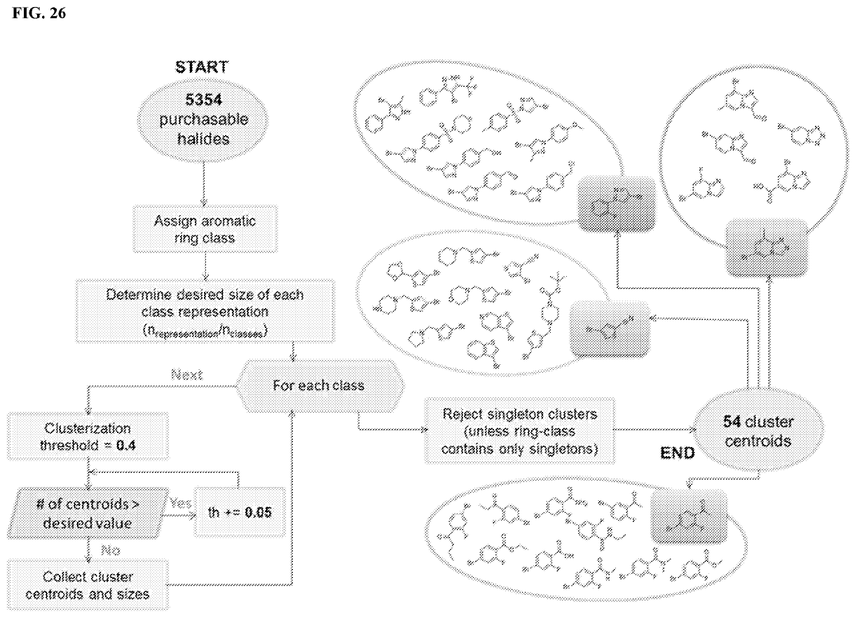

Data-guided down-selection of substrates. To enable practical pursuit of general hetero (aryl) SMC reaction conditions, strategic down-selecting of both the matrix of possible building block combinations and the matrix of possible reaction conditions occurs in a way that preserved relevance of the subsets to their wholes (as shown in step 202 of FIG. 2). Specifically, the inventories of common fine chemical suppliers were datamined and a list of ˜5400 (hetero)aryl halide building blocks were assembled that were practically purchasable and therefore accessible. To define a representative subset of this chemical space, a stratified clusterization strategy was applied (FIG. 26) to algorithmically cluster the building blocks by their common (hetero) aromatic ring substructures and pendant functionalities, down-selecting 54 “centroid” molecules most representative of each section of the available chemical space. Combining these molecules with a selection of 54 commercially available (hetero)aryl MIDA boronates defined a down-selected substrate scope composed of 2688 representative cross-coupling products (Figure S22 and S23). Mapping this potential product space and comparing it to all previously reported heteroaryl products in the literature reveals substantial overlap between both sets, suggesting that it is representative of heteroaryl chemical space as a whole (FIG. 3A). FIG. 3A illustrates a T-distributed stochastic neighbor embedding (t-SNE) mapping of the substrate combinations (here, 2688 heteroaryl products) examined, as compared to all (hetero)aryl products previously reported. Blue circles represent literature reported products; yellow stars represent products exclusively belonging in the reported search space; green triangles represent products present in both sets.

However, testing even this initially down-selected collection of cross-coupling products against many possible reaction conditions is technically unfeasible. Accordingly, a second layer of down-selection was pursued. Specifically, a greedy algorithm based on the Tanimoto similarity was used to identify from this larger collection a set of 11 representative substrate pairs that maximize mutual dissimilarity of the resulting products (FIG. 3B). For all of these products it was determined LCMS-UV/Vis response factor curves, which enabled the automatic determination the yields of automatically performed reactions. FIG. 3B illustrates t-SNE mapping of the product space synthesized during the training and test sets compared to the overall reaction space. Blue circles represent products belonging to the reported search space; yellow stars represent products belonging to the test set; green triangles represent products belonging to the training set.

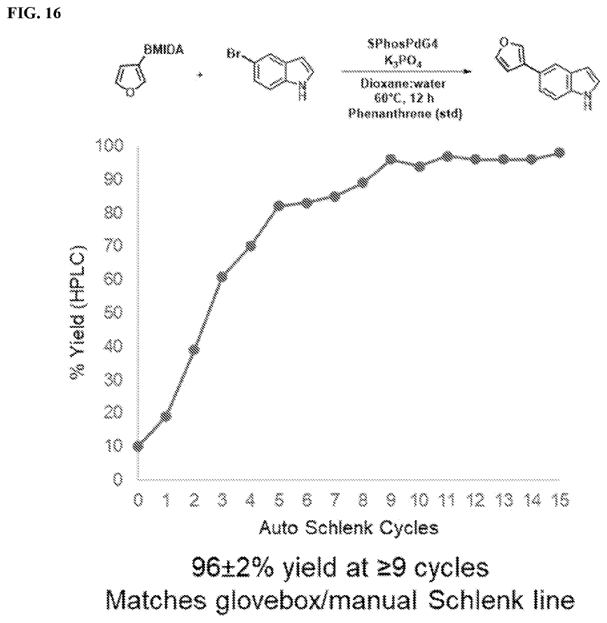

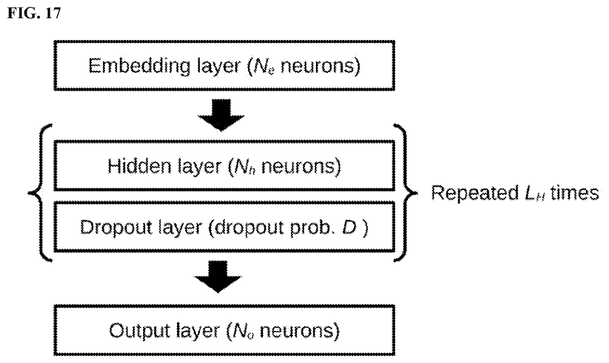

Data-guided down-selection of conditions. Regarding conditions, four variables were considered-solvent, base, catalyst/ligand and temperature. As the aim was to test a broad range of conditions, representatives of condition classes were initially down-selected based not only on their extent of prior use from our prior comprehensive literature analysis, but also structural and functional diversity. For instance, whereas two most commonly used solvents in the literature are dioxane and dimethoxyethane, they both belong to the same solvent class of ethers, and so only one was selected, dioxane. Similar reasoning led to keeping only one carbonate base. The temperature of 100° C. was as the most frequently used temperature in the literature, as well as 60° C. which was used in the previously developed benchmark protocol. In the end, three solvents were selected (dioxane, toluene, dimethylformamide, all used in 5:1 mixture with water), two bases (sodium carbonate, potassium phosphate), two temperatures (60° C. and 100° C.) and seven catalysts (Pd SPhos G4, Pd(PPh₃)₄, Pd XPhos G4, Pd P(tBu)₃ G4, Pd PCy3 G4, Pd₂ (dba)₃, and Pd(dppf)Cl₂. G4 refers to the fourth generation Buchwald precatalyst) to evaluate. The down-selected 11 building block combinations described above were tested under initial set of conditions to “seed” the ML optimization (FIG. 3C, illustrating a reaction scheme and chemical structures of an initial training set), and then iteratively under a much broader set of conditions during the ML-guided-optimization phase.
Seeding experiments, reaction standardization and conditions space. All reactions were performed automatically on a robotic system illustrated in FIG. 3D. Prior to solvent addition, heating, and stirring, reaction mixtures were purged with 10 automated vacuum/argon cycles, which led to highly reproducible reaction yields (FIG. 16). This automated Schlenk process was necessary—even when using air-stable precatalysts and building blocks—for reproducibility. To “seed” the optimization procedure, all couplings were performed between the aforementioned 11 pairs of substrates, each under seven different conditions: those corresponding to the JACS 2009 benchmark (5:1 Dioxane: water, 60° C., K₃PO₄, Pd SPhos G4), same base and solvents but with the other selected palladium catalysts (Pd XPhos G4, Pd P(tBu)₃ G4, Pd PCy3 G4, Pd₂(dba)₃, and Pd(dppf)Cl₂. G4, fourth generation Buchwald precatalyst), and a condition with the most common catalyst (Pd(PPh₃)₄), base (Na₂CO₃), temperature (100° C.), and solvent (dioxane: water) used in the literature (FIG. 3E). When each reaction was repeated twice, the yields exhibited only±2% deviation, underscoring one of the key advantages of automated experimentation (it has been reported that repetition of the same reaction even by the same human experts entails variability of ˜10-15%).

This initial round of experiments also allowed for the identification of catalysts that, for different substrate pairs, systematically gave similar yields and could thus be redundant. Such functional rather than structural similarity is quantified by the Spearman rank matrix shown in FIG. 3F and correlating yields obtained for all 11 substrate pairs using two different catalyst ligands—in this representation, redundant catalysts correspond to high-correlation, off-diagonal elements (e.g., XPhos and dppf, PCy3 and SPhos). Based on this analysis, PCy3 and dppf were eliminated from the pool of ligands, in order to decrease redundancy, and Pd₂ (dba) 3 was eliminated due to poor performance (<5% yield for 8/11 substrates), yielding a full space to be explored of 528 reactions (11 substrates×2 temperatures×2 bases×3 solvents×4 catalysts).

Uncertainty-minimizing ML for generality. A reaction condition can be considered maximally general when it provides the highest average yield across the widest range of chemical space. Optimization for generality is an unsolved and underexplored challenge in the evolving field of ML. An alternative approach was considered, where small sets of highly-reproducible data are generated on-demand during ML-guided closed-loop optimization, including negative data vastly underrepresented in existing datasets. The ML algorithm was also strategically focused on decreasing model uncertainty to thereby maximize the efficiency of the learning process.

Denoting the set of possible reaction conditions as C={c}, a set of substrate pairs as S={s} and reaction yield as y(s, c), the aim is to maximize the objective function given by:

$$f ( c ) = \frac{1}{\vert S \vert} {\sum }_{s ∈ S} y ( s , c ) \qquad ( 1 )$$

Then, the general conditions cgeneral are given as:

$${c}_{general} = arg {max}_{c ∈ C} f ( c ) \qquad ( 2 )$$

At first glance, the problem of identifying c_{general} in the least number of experiments resembles standard Bayesian optimization (BO). However, there is a substantial difference: in all BO algorithms, each experiment/measurement performed immediately provides information about the objective function desired for optimization. In contrast, experimental evaluation of f(c) in the given problem requires multiple experiments (because summation in eq. 1 runs over the entire set S)—that is, determination of f(c) for given conditions requires experiments with every pair of substrates in the S set. In order to address this problem, the standard BO approach was modified by constructing a surrogate model for predicting reaction yields, ŷ(s, c). Its predictions were then used to estimate \hat{f}(c) according to equation (1) and using the model's prediction for the yet-unperformed reactions. Note that in standard BO, f(c) would have been observed for the “seen” conditions and estimated for the “unseen” ones; in the present case f(c) is estimated even for the already tested conditions, unless the entire substrate space S had already been tested. Based on these considerations, the optimization over C (selection of the next conditions to examine) is performed with standard BO techniques, whereas sampling of S is achieved using an active learning approach. In particular, substrate pairs were chosen based on the model's prediction of uncertainty for given substrates under given reaction conditions: the highly uncertain (low-confidence) predictions indicate missing information, and providing the model with the corresponding experimental data decreases its uncertainty the most.

For uncertainty estimation, a model offering prediction uncertainty commensurate with prediction error was used—for instance, highly confident predictions with high error are undesirable. Based on the analyses of numerous neural-network, NN, as well as Gaussian Process, GP, models (SM Section 3), an ensemble of GP supplemented with a NN kernel component, GPE(NN) was chosen. Such a model is particularly appealing because of its flexibility (the similarity metric between different conditions will be learned from the data) and the reliability of the prediction uncertainties (e.g., it is guaranteed that the prediction of a test sample will not be more confident than a training sample). For the selection of conditions to be tested, the probability of improvement, PI, was chosen as an acquisition function.

Closed-loop, ML-driven optimization with robotic experimentation. The GPE(NN)/PI model guided the automated experiments over the down-selected search space. Multiple experiments were performed before the theoretical model was updated, creating “work batches.” Within each batch, the algorithm formed a “priority queue” of unexplored reactions by sorting and selecting conditions according to the computed PI and substrate pairs according to the prediction uncertainty. The batch size for rounds 1-2 of optimization was 36 duplicated reactions followed by 72 and 84 reactions for rounds 3-4 and round 5, respectively, not duplicated.

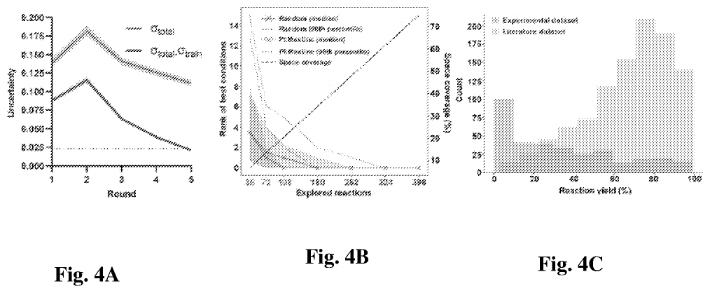

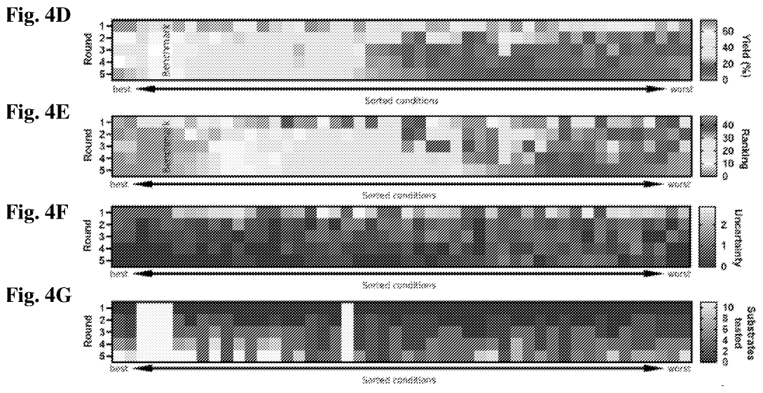

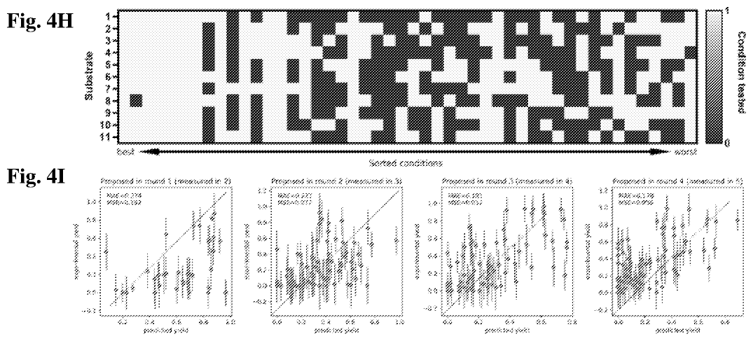

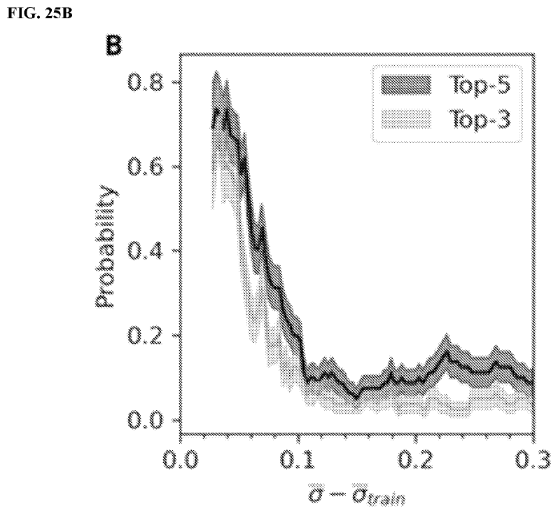

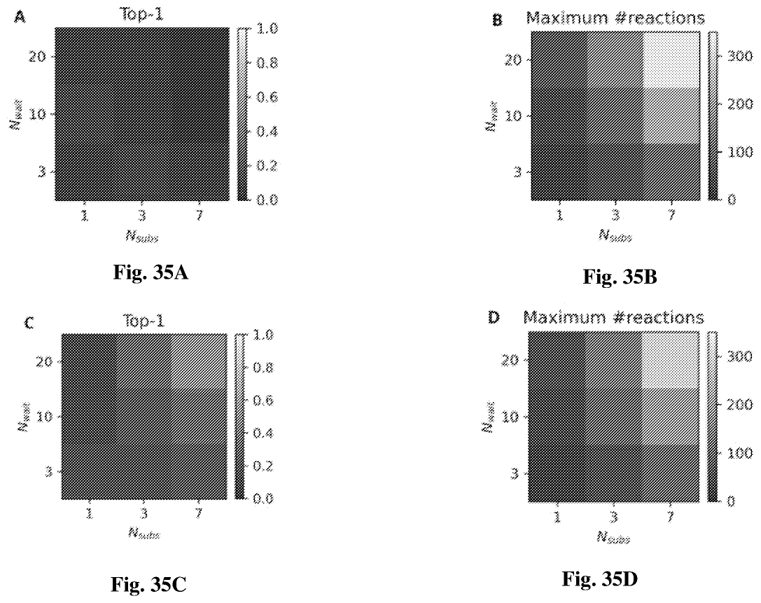

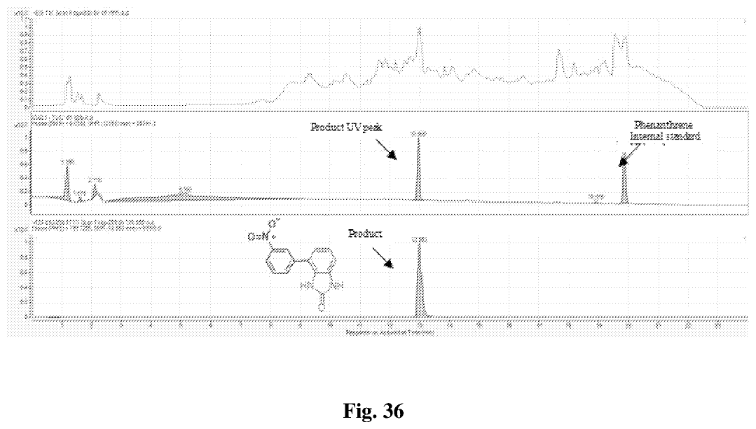

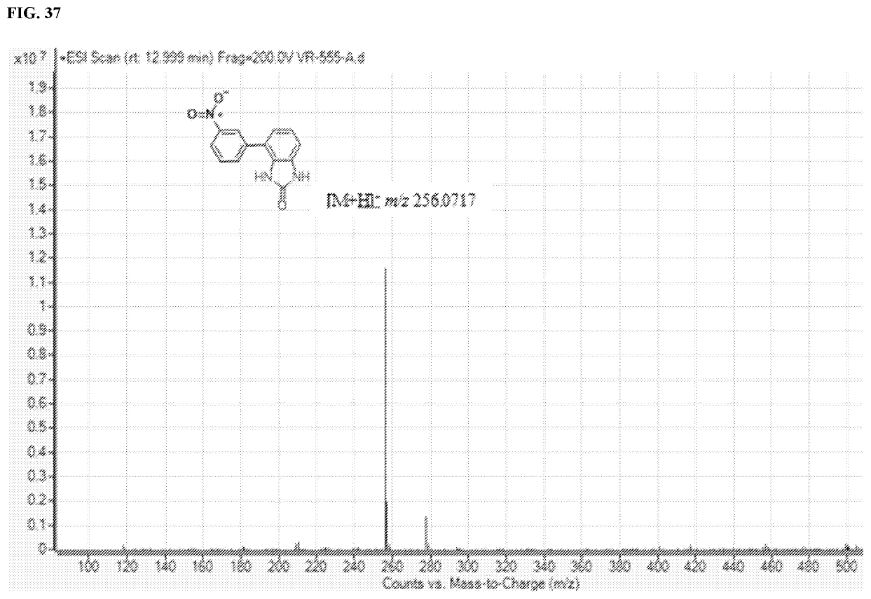

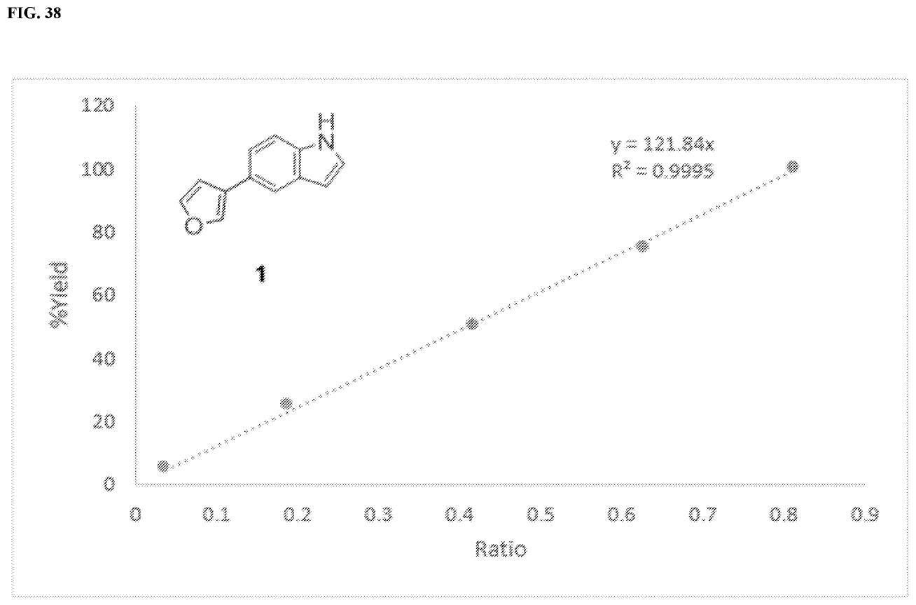

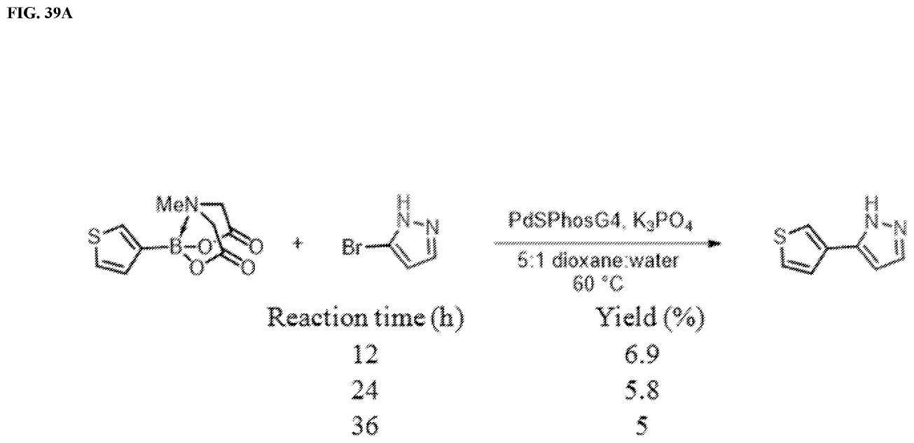

Over the closed-loop rounds, the model's uncertainty decreased and converged at the fifth round to the threshold obtained during calibration simulations (FIG. 4A, FIG. 25A, and FIG. 25B), suggesting that the model gained sufficient knowledge about the whole space, and at which point the optimization was terminated. FIG. 4A illustrates convergence of the model's uncertainty. Dashed horizontal line depicts threshold for σtotal-σtrain obtained in calibration simulations. The shaded areas correspond to 95% confidence interval computed by repeatedly training the model 10 times. This strategy converges to this optimum in about half as many reactions as random sampling (FIG. 4B), and with a higher likelihood of success compared to typical BO strategies (FIG. 35A-FIG. 35D). FIG. 4B illustrates a comparison of ML-guided vs. random searches for general conditions. Simulations with both model selection policy (Probability of Improvement, PI, in conditions space and Maximum Uncertainty, MaxUnc in substrate space for given conditions; abbreviated as PI: MaxUnc and corresponding to green lines) and random selection of the next reactions (red lines) were repeated 100 times in order to evaluate the random factor in the algorithm (random initialization of neural network weights as well as selection of the next step in the random baseline). The shaded areas mark the interquartile range.

As the algorithm explored the reaction-condition space, reaction yields for our dataset were distributed more or less uniformly over the range of possible values (FIG. 4C, comparing yield distribution between literature-reported reactions and those performed in the present disclosure). In other words, the protocol learned by probing both low- and high-yielding conditions. This should be contrasted with the distribution of yields in published reaction sets, where such yields are heavily skewed towards positive outcomes which limits the usefulness of approaches aiming to learn from published datasets.

In this dataset, the discovered top-1 condition conferred 72% average yield across all 11 substrates whereas the benchmark condition (found to also be the top-5 [fifth best] condition) conferred 64% average yield. To understand how the model arrived at this optimum, the model's perception of the average yield and ranking of each general condition per round was examined, as shown in FIGS. 4D-E. As shown in FIG. 4D, within the first two rounds, the model gains the ability to accurately categorize these conditions into high, medium, and low overall average yield, and, in the subsequent rounds, establishes the correct ranking within these categories. In FIG. 4E the ranking per general condition per round is illustrated as perceived by the ML model. The increasing accuracy of the model over the course of the experiment is recapitulated in FIG. 4F which shows the model's ranking uncertainty decreasing between rounds and is especially apparent for the top conditions. The model chose to test a few substrates per round across many conditions for the first 3 rounds, followed by primarily filling in the top conditions in the latter rounds (FIG. 4G). By the fifth round, the model explored nearly all of the top 7 conditions, which corresponds to every condition with >50% overall average yield as estimated by the model (FIG. 4H).

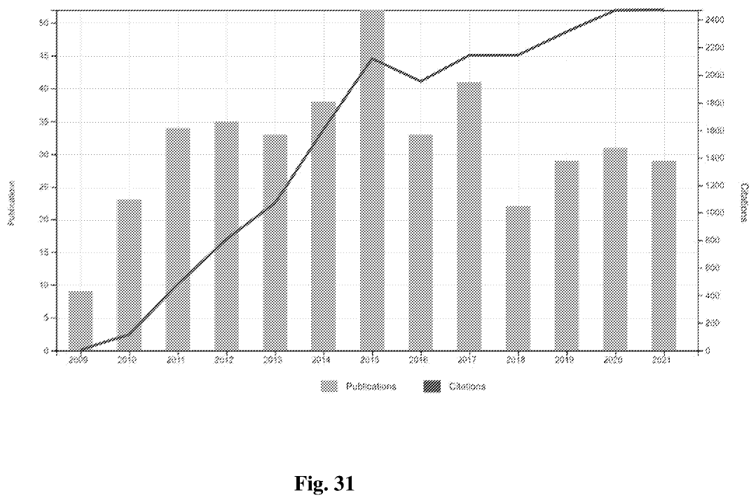

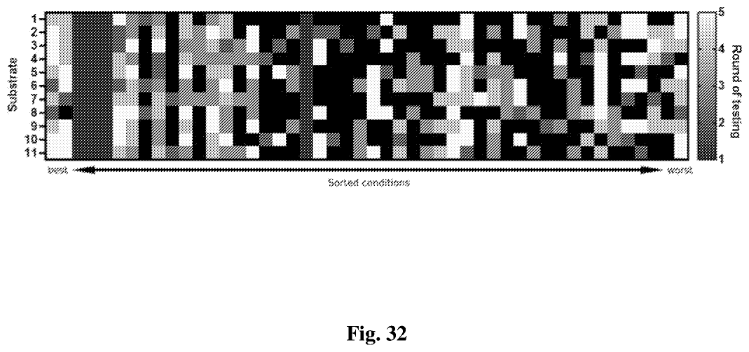

Finally, the yields of reactions the model requested were analyzed in order to gain more information about the reaction-condition space (FIG. 31). These values are not expected to increase as the optimization progresses, since the yield of a single experiment is not our objective. Given the uncertainty-guided selection of substrates, the opposite could be expected t: once a set of suitable conditions is identified, further exploitation should involve lower-yielding reactions in order to verify that the found candidate conditions are indeed better—as well as to increase confidence of the estimate of f(c). The results shown in FIG. 3I indicate that after exploring good reactions in the second iteration, the model gradually shifted its attention towards these parts of the reaction-condition space that can be considered as ‘negative examples’ (and in doing so, improved its prediction accuracy). From these results, it appears that i) relatively good candidate solutions were identified early, ii) the model initially tried to look for better yielding reactions (to find better candidates) and iii) more and more attention was dedicated to decreasing the uncertainty of its estimates as the ‘loop’ progressed.

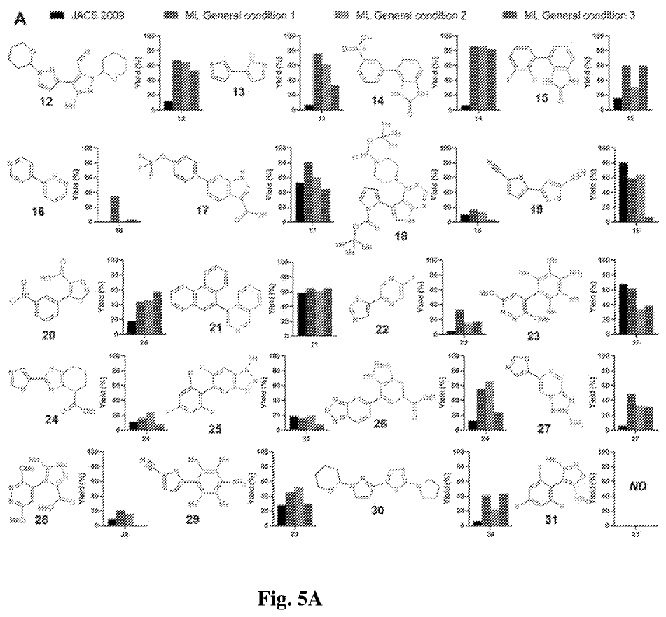

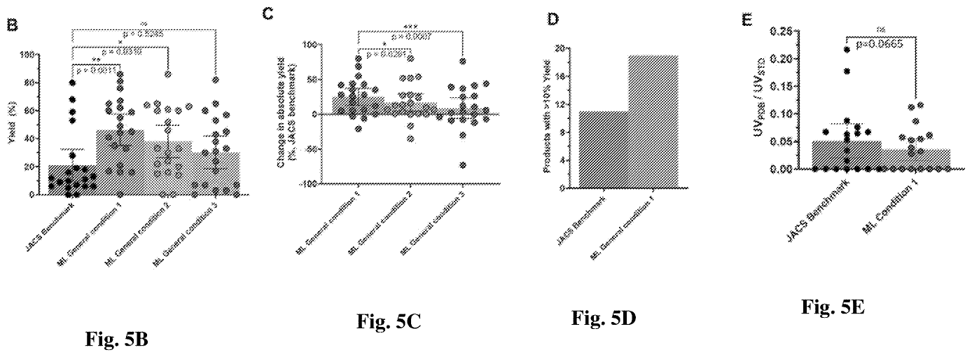

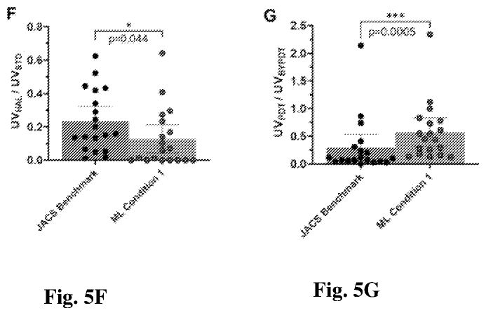

Quantifying generality. Following the discovery of higher-yielding general conditions within the training set, we next sought to interrogate whether the learning would transfer to substrates outside of the optimization—specifically, over 20 substrate pairs chosen (by the Butina (37) algorithm) to maximize dissimilarity to the training set while ensuring coverage of the heterocyclic substructure and functional group space (FIG. 3B). We then committed to synthesizing and purifying all of the computer's suggestions and tested them against the benchmark condition and the top 3 highest yielding general reaction conditions discovered during the closed-loop optimization (FIG. 5A), as ranked by the model after the completion of round 5. Despite including some very challenging building block combinations, this process was 95% successful with only one product having no measurable yield under all 4 conditions.

The ML-discovered general reaction conditions performed remarkably better than the previously reported and widely used benchmark condition (14). The top 2 conditions provided statistically significant increases in average yield compared to the benchmark, with the top condition doubling the overall average yield from 21% to 46% (FIG. 4B). Comparing the relative increase in yield reveals statistically significant differences between the top-1 and both the top-2 and top-3 conditions (FIG. 5C). Remarkably, the experimental yields correlate with the predicted ranking of the conditions such that the yield for the top-1 is higher than the top-2, which, in turn, is higher than the top-3. In the context of functional discovery efforts, the binary capacity to isolate or not isolate testable quantities of purified targeted compounds is arguably even more important than the specific percent yield. We estimate the practical limit for isolating purified products is 10% yield. For the benchmark condition only 11/20 targeted products cleared this bar, whereas this fraction rose to 19/20 for the top-1 condition (FIG. 4D).

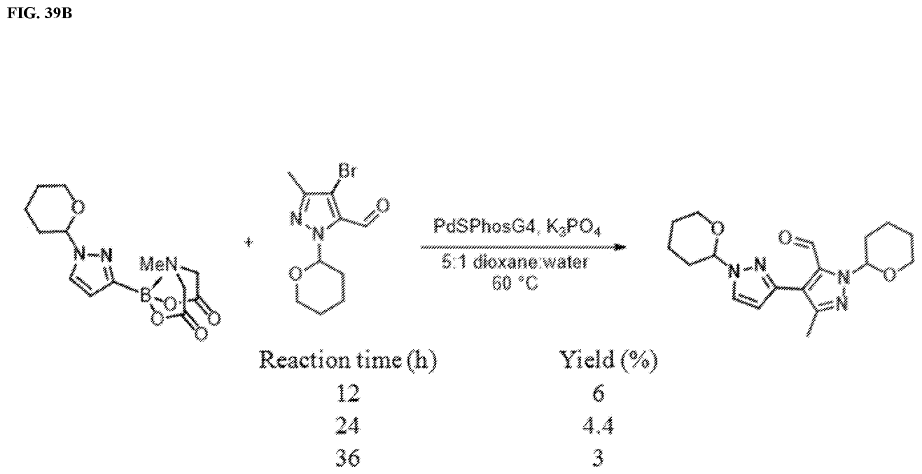

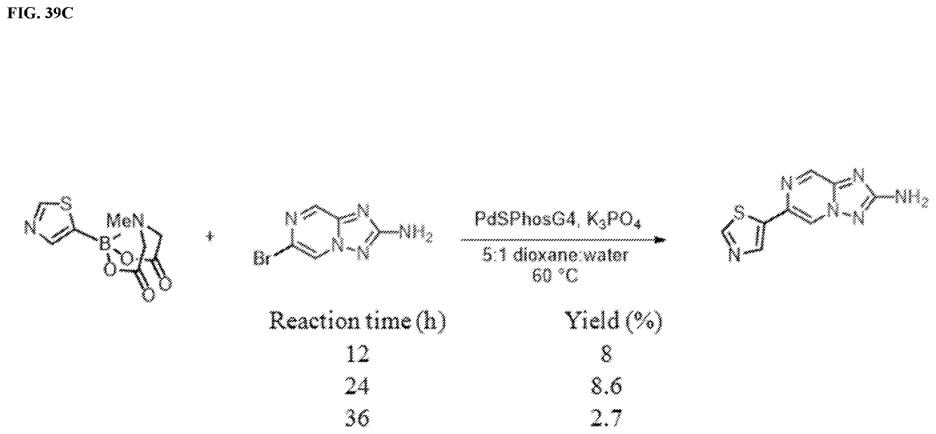

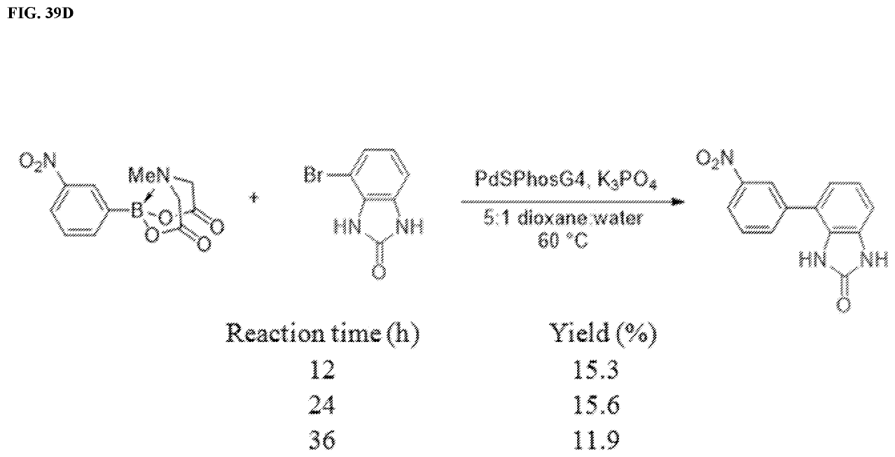

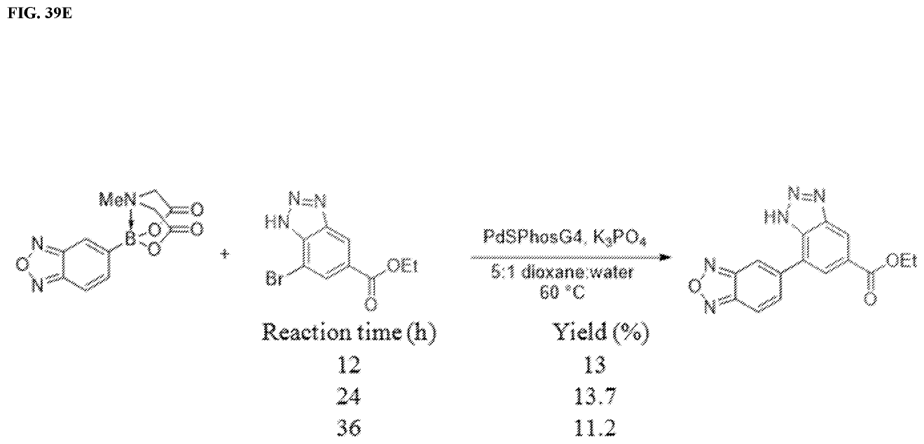

It is noted that extending the reaction times for couplings that were low yielding under the benchmark conditions did not increase yields (FIG. 39A-FIG. 39E). Comprehensive analysis of byproducts and product formation for all 20 reactions demonstrated that a favorable shift from the former to the latter accompanies the shift from the benchmark to the ML-discovered reaction conditions. Specifically, the ML-discovered conditions were associated with a trend toward decreased protodeboronation (FIG. 5E), increased halide conversion (FIG. 5F), and an overall statistically significant increase in the ratio of product to total byproducts formation (FIG. 5G) (0.30±0.12 for JACS vs. 0.58±0.12 for ML conditions, p=0.0005).

The straightforward workflow developed here has enabled the accelerated discovery of improved general reaction conditions for difficult C—C bond forming reactions, representing a key step towards increasing the efficiency, generality, and accessibility of small molecule synthesis. This result also highlights the power of down-selection as an entry point into large multidimensional search spaces, the distinct advantages of a de novo ML approach for navigating such spaces by generating data sets that evenly reflect the reality of positive and negative data during optimization, and the unique suitability of robotized chemistry for generating high quality, reproducible data.

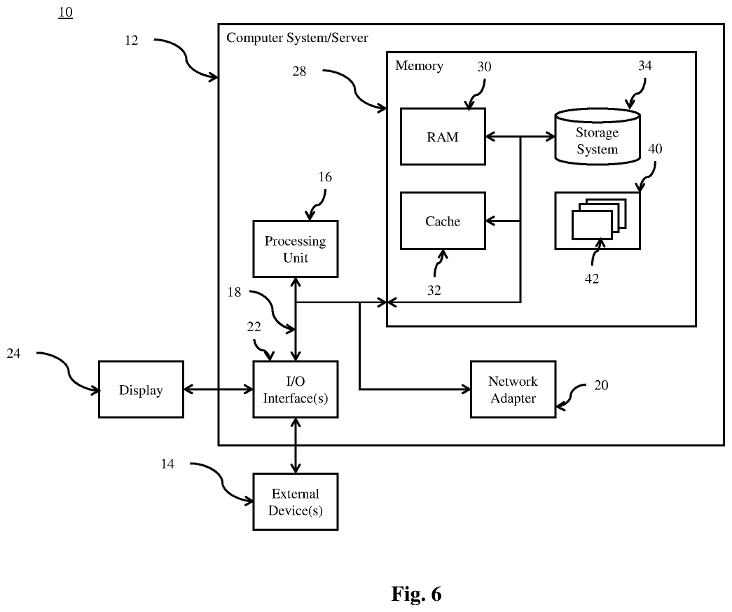

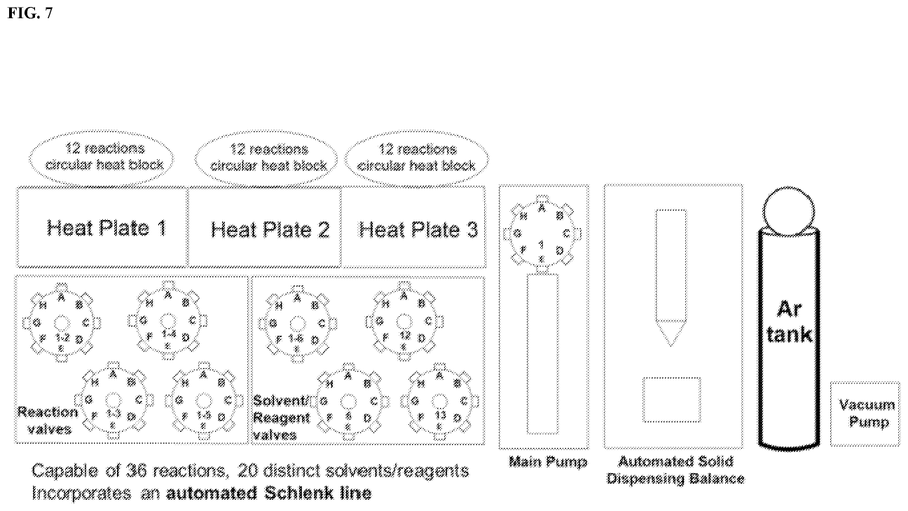

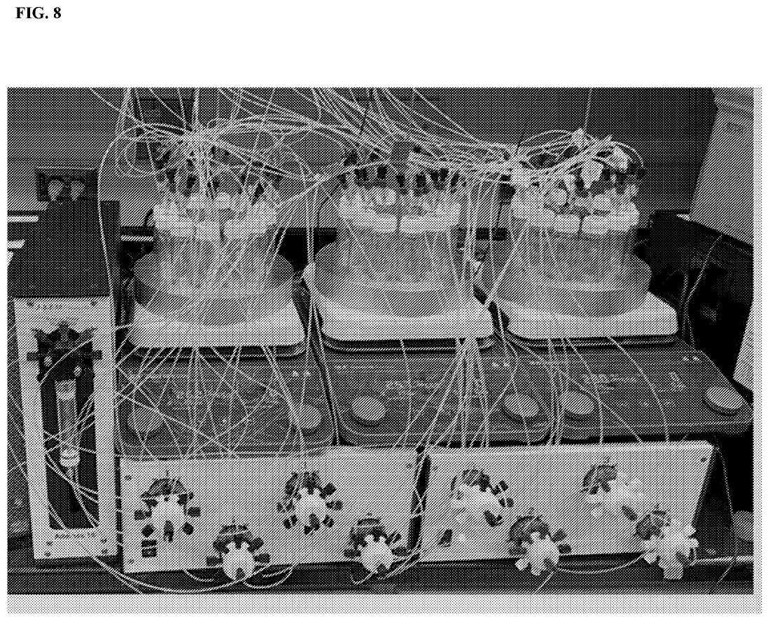

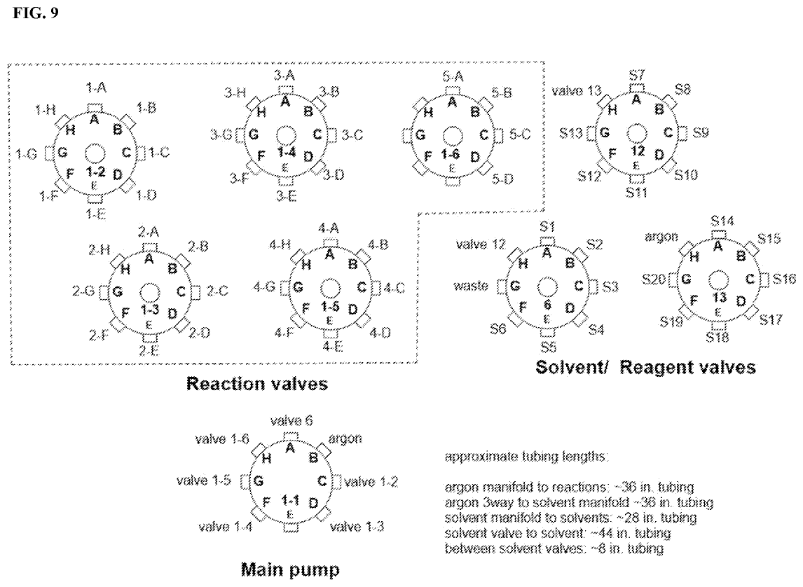

Computing Nodes. Referring now to FIG. 6, a schematic of an example of a computing node is shown. Computing node 10 is only one example of a suitable computing node and is not intended to suggest any limitation as to the scope of use or functionality of embodiments described herein. Regardless, computing node 10 is capable of being implemented and/or performing any of the functionality set forth hereinabove.

In computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 6, computer system/server 12 in computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, Peripheral Component Interconnect (PCI) bus, Peripheral Component Interconnect Express (PCIe), and Advanced Microcontroller Bus Architecture (AMBA).

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the disclosure.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

The present disclosure may be embodied as a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

### Definitions

As used herein, the term “greedy algorithm” refers to any algorithm that follows the problem-solving heuristic of making the locally optimal choice at each stage.

The term “organic moiety” as used herein refers to a singly-valent group containing one or more carbon atoms. The organic moiety may be aromatic or may be derived from a hydrocarbon. The organic moiety may comprise one or more heteroatoms, one or more units of unsaturation, and/or one or more functional groups. The organic moiety may be substituted.

The term “alkyl” as used herein is a term of art and refers to saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight-chain or branched-chain alkyl has about 30 or fewer carbon atoms in its backbone (e.g., C₁-C₃₀ for straight chain, C₃-C₃₀ for branched chain), and alternatively, about 20 or fewer, or 10 or fewer. In certain embodiments, the term “alkyl” refers to a C₁-C₁₀ alkyl group. In certain embodiments, the term “alkyl” refers to a C₁-C₆ alkyl group, for example a C₁-C₆ straight-chain alkyl group. In certain embodiments, the term “alkyl” refers to a C₃-C₁₂ branched-chain alkyl group. In certain embodiments, the term “alkyl” refers to a C₃-C₈ branched-chain alkyl group. Representative examples of alkyl include, but are not limited to, methyl, ethyl, n-propyl, iso-propyl, n-butyl, sec-butyl, iso-butyl, tert-butyl, n-pentyl, isopentyl, neopentyl, and n-hexyl.

The term “cycloalkyl” means mono- or bicyclic or bridged saturated carbocyclic rings, each having from 3 to 12 carbon atoms. Certain cycloalkyls have from 5-12 carbon atoms in their ring structure, and may have 6-10 carbons in the ring structure. Preferably, cycloalkyl is (C₃-C₇) cycloalkyl, which represents a monocyclic saturated carbocyclic ring, having from 3 to 7 carbon atoms. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems include bridged monocyclic rings and fused bicyclic rings. Bridged monocyclic rings contain a monocyclic cycloalkyl ring where two non-adjacent carbon atoms of the monocyclic ring are linked by an alkylene bridge of between one and three additional carbon atoms (i.e., a bridging group of the form —(CH₂)_{w}—, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. Fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. The bridged or fused bicyclic cycloalkyl is attached to the parent molecular moiety through any carbon atom contained within the monocyclic cycloalkyl ring. Cycloalkyl groups are optionally substituted. In certain embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted.

The term “(cycloalkyl)alkyl” as used herein refers to an alkyl group substituted with one or more cycloalkyl groups. An example of (cycloalkyl)alkyl is cyclohexylmethyl group.

The term “heterocycloalkyl” as used herein refers to a radical of a non-aromatic ring system, including, but not limited to, monocyclic, bicyclic, and tricyclic rings, which can be completely saturated or which can contain one or more units of unsaturation, for the avoidance of doubt, the degree of unsaturation does not result in an aromatic ring system, and having 3 to 12 atoms including at least one heteroatom, such as nitrogen, oxygen, or sulfur. For purposes of exemplification, which should not be construed as limiting the scope of this invention, the following are examples of heterocyclic rings: aziridinyl, azirinyl, oxiranyl, thiiranyl, thiirenyl, dioxiranyl, diazirinyl, diazepanyl, 1,3-dioxanyl, 1,3-dioxolanyl, 1,3-dithiolanyl, 1,3-dithianyl, imidazolidinyl, isothiazolinyl, isothiazolidinyl, isoxazolinyl, isoxazolidinyl, azetyl, oxetanyl, oxetyl, thietanyl, thietyl, diazetidinyl, dioxetanyl, dioxetenyl, dithietanyl, dithietyl, dioxalanyl, oxazolyl, thiazolyl, triazinyl, isothiazolyl, isoxazolyl, azepines, azetidinyl, morpholinyl, oxadiazolinyl, oxadiazolidinyl, oxazolinyl, oxazolidinyl, oxopiperidinyl, oxopyrrolidinyl, piperazinyl, piperidinyl, pyranyl, pyrazolinyl, pyrazolidinyl, pyrrolinyl, pyrrolidinyl, quinuclidinyl, thiomorpholinyl, tetrahydropyranyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. A heterocycloalkyl group is optionally substituted by one or more substituents as described below.

The term “(heterocycloalkyl)alkyl” as used herein refers to an alkyl group substituted with one or more heterocycloalkyl (i.e., heterocyclyl) groups.

The term “alkenyl” as used herein means a straight or branched chain hydrocarbon radical containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl. The unsaturated bond(s) of the alkenyl group can be located anywhere in the moiety and can have either the (Z) or the (E) configuration about the double bond(s).

The term “alkynyl” as used herein means a straight or branched chain hydrocarbon radical containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “alkylene” is art-recognized, and as used herein pertains to a diradical obtained by removing two hydrogen atoms of an alkyl group, as defined above. In one embodiment an alkylene refers to a disubstituted alkane, i.e., an alkane substituted at two positions with substituents such as halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, fluoroalkyl (such as trifluromethyl), cyano, or the like. That is, in one embodiment, a “substituted alkyl” is an “alkylene”.

The term “amino” is a term of art and as used herein refers to both unsubstituted and substituted amines, e.g., a moiety that may be represented by the general formulas:

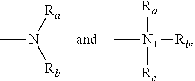

wherein Rₐ, R_{b}, and R_{c} each independently represent a hydrogen, an alkyl, an
alkenyl, —(CH₂)ₓ—R_{d}, or Rₐ and R_{b}, taken together with the N atom to which they are attached complete a heterocycle having from 4 to 8 atoms in the ring structure; R_{d} represents an aryl, a cycloalkyl, a cycloalkenyl, a heterocyclyl or a polycyclyl; and x is zero or an integer in the range of 1 to 8. In certain embodiments, only one of Rₐ or R_{b} may be a carbonyl, e.g., Rₐ, R_{b}, and the nitrogen together do not form an imide. In other embodiments, Rₐ and R_{b} (and optionally R_{c}) each independently represent a hydrogen, an alkyl, an alkenyl, or —(CH₂)ₓ—R_{d}. In certain embodiments, the term “amino” refers to —NH₂.

In certain embodiments, the term “alkylamino” refers to —NH(alkyl).

In certain embodiments, the term “dialkylamino” refers to —N(alkyl)₂.

The term “amido”, as used herein, means —NHC(═O)—, wherein the amido group is bound to the parent molecular moiety through the nitrogen. Examples of amido include alkylamido such as CH₃C(═O)N(H)— and CH₃CH₂C(═O)N(H)—.

The term “acyl” is a term of art and as used herein refers to any group or radical of the form RCO— where R is any organic group, e.g., alkyl, aryl, heteroaryl, aralkyl, and heteroaralkyl. Representative acyl groups include acetyl, benzoyl, and malonyl.

The term “aminoalkyl” as used herein refers to an alkyl group substituted with one or more one amino groups. In one embodiment, the term “aminoalkyl” refers to an aminomethyl group.

The term “aminoacyl” is a term of art and as used herein refers to an acyl group substituted with one or more amino groups.

The term “aminothionyl” as used herein refers to an analog of an aminoacyl in which the O of RC(O)— has been replaced by sulfur, hence is of the form RC(S)—.

The term “phosphoryl” is a term of art and as used herein may in general be represented by the formula:

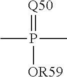

wherein Q50 represents S or O, and R59 represents hydrogen, a lower alkyl or an aryl; for example, —P(O)(OMe)— or —P(O)(OH)₂. When used to substitute, e.g., an alkyl, the phosphoryl group of the phosphorylalkyl may be represented by the general formulas:

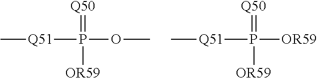

wherein Q50 and R59, each independently, are defined above, and Q51 represents O, S or N; for example, —O—P(O)(OH)OMe or —NH—P(O)(OH)₂. When Q50 is S, the phosphoryl moiety is a “phosphorothioate.”

The term “aminophosphoryl” as used herein refers to a phosphoryl group substituted with at least one amino group, as defined herein; for example, —P(O)(OH)NMe₂.

The term “azide” or “azido”, as used herein, means an —N₃ group.

The term “carbonyl” as used herein refers to —C(═O)—.

The term “thiocarbonyl” as used herein refers to —C(═S)—.

The term “alkylphosphoryl” as used herein refers to a phosphoryl group substituted with at least one alkyl group, as defined herein; for example, —P(O)(OH)Me.

The term “alkylthio” as used herein refers to alkyl —S—. The term “(alkylthio)alkyl” refers to an alkyl group substituted by an alkylthio group.

The term “carboxy”, as used herein, means a —CO₂H group.

The term “aryl” is a term of art and as used herein refers to includes monocyclic, bicyclic and polycyclic aromatic hydrocarbon groups, for example, benzene, naphthalene, anthracene, and pyrene. Typically, an aryl group contains from 6-10 carbon ring atoms (i.e., (C₆-C₁₀)aryl). The aromatic ring may be substituted at one or more ring positions with one or more substituents, such as halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, fluoroalkyl (such as trifluromethyl), cyano, or the like. The term “aryl” also includes polycyclic ring systems having two or more cyclic rings in which two or more carbons are common to two adjoining rings (the rings are “fused rings”) wherein at least one of the rings is an aromatic hydrocarbon, e.g., the other cyclic rings may be cycloalkyls, cycloalkenyls, cycloalkynyls, aryls, heteroaryls, and/or heterocyclyls. In certain embodiments, the term “aryl” refers to a phenyl group.

The term “arylene” means a diradical obtained by removing two hydrogen atoms of an aryl group, as defined above. In certain embodiments an arylene refers to a disubstituted arene, i.e., an arene substituted at two positions with substituents such as halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, fluoroalkyl (such as trifluoromethyl), cyano, or the like. That is, in certain embodiments, a “substituted aryl” is an “arylene”.

The term “heteroaryl” is a term of art and as used herein refers to a monocyclic, bicyclic, and polycyclic aromatic group having 3 to 12 total atoms including one or more heteroatoms such as nitrogen, oxygen, or sulfur in the ring structure. Exemplary heteroaryl groups include azaindolyl, benzo (b) thienyl, benzimidazolyl, benzofuranyl, benzoxazolyl, benzothiazolyl, benzothiadiazolyl, benzotriazolyl, benzoxadiazolyl, furanyl, imidazolyl, imidazopyridinyl, indolyl, indolinyl, indazolyl, isoindolinyl, isoxazolyl, isothiazolyl, isoquinolinyl, oxadiazolyl, oxazolyl, purinyl, pyranyl, pyrazinyl, pyrazolyl, pyridinyl, pyrimidinyl, pyrrolyl, pyrrolo[2,3-d]pyrimidinyl, pyrazolo[3,4-d]pyrimidinyl, quinolinyl, quinazolinyl, triazolyl, thiazolyl, thiophenyl, tetrahydroindolyl, tetrazolyl, thiadiazolyl, thienyl, thiomorpholinyl, triazolyl or tropanyl, and the like. The “heteroaryl” may be substituted at one or more ring positions with one or more substituents such as halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, fluoroalkyl (such as trifluromethyl), cyano, or the like. The term “heteroaryl” also includes polycyclic ring systems having two or more cyclic rings in which two or more carbons are common to two adjoining rings (the rings are “fused rings”) wherein at least one of the rings is an aromatic group having one or more heteroatoms in the ring structure, e.g., the other cyclic rings may be cycloalkyls, cycloalkenyls, cycloalkynyls, aryls, heteroaryls, and/or heterocyclyls.

The term “heteroarylene” means a diradical obtained by removing two hydrogen atoms of a heteroaryl group, as defined above. In certain embodiments an heteroarylene refers to a disubstituted heteroarene, i.e., a heteroarene substituted at two positions with substituents such as halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, fluoroalkyl (such as trifluoromethyl), cyano, or the like. That is, in certain embodiments, a “substituted heteroaryl” is an “heteroarylene”.

The term “aralkyl” or “arylalkyl” is a term of art and as used herein refers to an alkyl group substituted with an aryl group, wherein the moiety is appended to the parent molecule through the alkyl group.

The term “heteroaralkyl” or “heteroarylalkyl” is a term of art and as used herein refers to an alkyl group substituted with a heteroaryl group, appended to the parent molecular moiety through the alkyl group.

The term “alkoxy” as used herein means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkoxyalkyl” refers to an alkyl group substituted by an alkoxy group.

The term “alkoxycarbonyl” means an alkoxy group, as defined herein, appended to the parent molecular moiety through a carbonyl group, represented by —C(═O)—, as defined herein. Representative examples of alkoxycarbonyl include, but are not limited to, methoxycarbonyl, ethoxycarbonyl, and tert-butoxycarbonyl.

The term “alkylcarbonyl”, as used herein, means an alkyl group, as defined herein, appended to the parent molecular moiety through a carbonyl group, as defined herein. Representative examples of alkylcarbonyl include, but are not limited to, acetyl, 1-oxopropyl, 2,2-dimethyl-1-oxopropyl, 1-oxobutyl, and 1-oxopentyl.

The term “arylcarbonyl”, as used herein, means an aryl group, as defined herein, appended to the parent molecular moiety through a carbonyl group, as defined herein. Representative examples of arylcarbonyl include, but are not limited to, benzoyl and (2-pyridinyl) carbonyl.

The term “alkylcarbonyloxy” and “arylcarbonyloxy”, as used herein, means an alkylcarbonyl or arylcarbonyl group, as defined herein, appended to the parent molecular moiety through an oxygen atom. Representative examples of alkylcarbonyloxy include, but are not limited to, acetyloxy, ethylcarbonyloxy, and tert-butylcarbonyloxy. Representative examples of arylcarbonyloxy include, but are not limited to phenylcarbonyloxy.

The term “alkenoxy” or “alkenoxyl” means an alkenyl group, as defined herein, appended to the parent molecular moiety through an oxygen atom. Representative examples of alkenoxyl include, but are not limited to, 2-propen-1-oxyl (i.e., CH₂═CH—CH₂—O—) and vinyloxy (i.e., CH₂═CH—O—).

The term “aryloxy” as used herein means an aryl group, as defined herein, appended to the parent molecular moiety through an oxygen atom.

The term “heteroaryloxy” as used herein means a heteroaryl group, as defined herein, appended to the parent molecular moiety through an oxygen atom.

The term “carbocyclyl” as used herein means a monocyclic or multicyclic (e.g., bicyclic, tricyclic, etc.) hydrocarbon radical containing from 3 to 12 carbon atoms that is completely saturated or has one or more unsaturated bonds, and for the avoidance of doubt, the degree of unsaturation does not result in an aromatic ring system (e.g., phenyl). Examples of carbocyclyl groups include 1-cyclopropyl, 1-cyclobutyl, 2-cyclopentyl, 1-cyclopentenyl, 3-cyclohexyl, 1-cyclohexenyl and 2-cyclopentenylmethyl.

The term “cyano” is a term of art and as used herein refers to —CN.

The term “halo” is a term of art and as used herein refers to —F, —Cl, —Br, or —I.

The term “haloalkyl” as used herein refers to an alkyl group, as defined herein, wherein some or all of the hydrogens are replaced with halogen atoms.

The term “hydroxy” is a term of art and as used herein refers to —OH.

The term “hydroxyalkyl”, as used herein, means at least one hydroxy group, as defined herein, is appended to the parent molecular moiety through an alkyl group, as defined herein. Representative examples of hydroxyalkyl include, but are not limited to, hydroxymethyl, 2-hydroxyethyl, 3-hydroxypropyl, 2,3-dihydroxypentyl, and 2-ethyl-4-hydroxyheptyl.

The term “silyl”, as used herein, includes hydrocarbyl derivatives of the silyl (H₃Si—) group (i.e., (hydrocarbyl)₃Si—), wherein a hydrocarbyl groups are univalent groups formed by removing a hydrogen atom from a hydrocarbon, e.g., ethyl, phenyl. The hydrocarbyl groups can be combinations of differing groups which can be varied in order to provide a number of silyl groups, such as trimethylsilyl (TMS), tert-butyldiphenylsilyl (TBDPS), tert-butyldimethylsilyl (TBS/TBDMS), triisopropylsilyl (TIPS), and [2-(trimethylsilyl) ethoxy]methyl (SEM).

The term “silyloxy”, as used herein, means a silyl group, as defined herein, is appended to the parent molecule through an oxygen atom.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, fragmentation, decomposition, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

In certain embodiments, the optional substituents can include, for example, halogen, haloalkyl, hydroxyl, carbonyl (such as carboxyl (—COOH), alkoxycarbonyl, formyl, or acyl), thiocarbonyl (such as thioester, thioacetate, or thioformate), alkoxyl, alkenyloxy, alkynyloxy, phosphoryl, phosphate, phosphonate, phosphinate, amino (including alkyl- and dialkylamino), amido, amidine, imine, cyano, nitro, oxo (═O), azido, sulfhydryl, alkylthio, sulfate, sulfonate, sulfamoyl, sulfonamido, sulfonyl, silyl, silyloxy, heterocycloalkyl, cycloalkyl, alkyl, alkenyl, alkynyl, aryl, heteroaryl, arylalkyl, or heteroarylalkyl group.

For purposes of the invention, the chemical elements are identified in accordance with the Periodic Table of the Elements, CAS version, Handbook of Chemistry and Physics, 67th Ed., 1986-87, inside cover.

Other chemistry terms herein are used according to conventional usage in the art, as exemplified by The McGraw-Hill Dictionary of Chemical Terms (ed. Parker, S., 1985), McGraw-Hill, San Francisco, incorporated herein by reference). Unless otherwise defined, all technical and scientific terms used herein have the same meaning as commonly understood by one of ordinary skill in the art to which this invention pertains.

In the compounds of this invention any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Also unless otherwise stated, when a position is designated specifically as “D” or “deuterium”, the position is understood to have deuterium at an abundance that is at least 3340 times greater than the natural abundance of deuterium, which is 0.015% (i.e., at least 50.1% incorporation of deuterium).

The term “isotopic enrichment factor” as used herein means the ratio between the isotopic abundance and the natural abundance of a specified isotope.

In various embodiments, compounds of this invention have an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

### EXAMPLES

The invention now being generally described, it will be more readily understood by reference to the following examples which are included merely for purposes of illustration of certain aspects and embodiments of the present invention, and are not intended to limit the invention.

### Example 1—Exemplary Materials and Methods

Materials. Commercial reagents were purchased from Sigma-Aldrich, Combi-Blocks, TCI America, Fisher Scientific, Oakwood Products, or Strem and were used without further purification unless otherwise noted. Solvents (1,4-dioxane, DMF, and Toluene) were purchased from Fisher Scientific and used in reaction after one hour purging of argon. Deionized water was purified through a Milli-Q water filtration system. Na₂CO₃ and K₃PO₄ were both anhydrous and used as purchased from suppliers.

Characterization. Proton nuclear magnetic resonance (¹H NMR) spectra were recorded at room temperature on one of the following instruments: Bruker 500-MHz spectrometer with broad-band cryoprobe (CB500) or Bruker Avance Neo 600 MHz spectrometer with a Prodigy BBQ-BB cryoprobe (B600). Chemical shifts (δ) are reported in parts per million (ppm) downfield from tetramethylsilane and referenced to residual protium in the NMR solvent (CDCl₃, δ=7.26 ppm, center line; (CD₃)₂CO, δ=2.05 ppm, center line; (CD₃)₂SO, δ=2.50 ppm, center line; CD₃OD, δ=3.31 ppm, center line). Data are reported as follows: chemical shift, multiplicity (s=singlet, d=doublet, t=triplet, q=quartet, quint=quintet, sept=septet, m=multiplet, dd=doublet of doublets, dt=doublet of triplets, dq=doublet of quartets), coupling constant (J) in Hertz (Hz), and integration. ¹³C NMR spectra were recorded at room temperature on one of the following instruments: CB500 and B600. Chemical shifts (δ) are reported in ppm downfield from tetramethylsilane and referenced to carbon resonances in the NMR solvent (CDCl₃, δ=77.0 ppm, center line; (CD₃)₂CO, δ=29.84 ppm, center line; (CD₃)₂SO, δ=39.52, ppm center line; CD₃OD, δ=49.00 ppm, center line). Chemical shifts for fluorine nuclear magnetic resonance (¹⁹F NMR) are reported in parts per million downfield from chlorotrifluoromethane (δ=0). High resolution mass spectra (HRMS) using TOF-MS were performed by Furong Sun and Haijun Yao at the University of Illinois School of Chemical Sciences Mass Spectrometry Laboratory. Liquid Chromatography—Mass Spectrometry (LCMS) was performed using an Agilent 1260 Infinity II HPLC with diode array UV-Vis detector (DAD) connected to an Agilent 6530C quadropole time-of-flight (QTOF) MS detector. For LCMS, a YMC America Triart C18 150 mm length×4.6 mm internal diameter 120 Å pore size 5 μm particle size (model number: TA12S05-1546WT) column was utilized and 1 μL of undiluted crude reaction mixtures were injected for quantification under standard separation conditions. Standard separation conditions for all molecules (except for product 27 where peaks co-eluted) were: column compartment set to 25° C., 1 mL/min flowrate of A: Water with 0.1% formic acid and B: LCMS grade acetonitrile using gradient: 0→1 min 100% A 0% B, 1→20 min 0% A 100% B, 20→22 min 0% A 100% B, 20→22 min 0% A 100% B, 22→23 min 95% A 5% B, 23→24 min 100% A 0% B, with DAD measuring signals: 200 nm, 230 nm, 250 nm, 270 nm, 290 nm, 310 nm, 380 nm, 430 nm, each with 4 nm bandwidth. Separation conditions for product 27 were the same as above except with modification to the gradient: 0→1 min 100% A 0% B, 1→20 min 60% A 40% B, 20→21 min 0% A 100% B, 21→27 min 0% A 100% B, 27→28 min 100% A 0% B, 28→29 min 100% A 0% B.

Purification. Preparative high-performance liquid chromatography (prep HPLC) was performed on an Agilent 1200 series instrument with a Waters SunFire Prep C18 OBD 5 μM 30×150 mm column.

Automated Synthesis. All reactions were performed in 40 mL glass I-Chem vials with PTFE lined-septa cap (Fisher Scientific catalogue no. 05-719-106) equipped with a rare earth stir bar (oval, diameter 5 mm, length 10 mm, Sigma-Aldrich catalogue no. Z671622) under a positive pressure of argon using an automated synthesis machine. Solid chemicals (catalyst, base, building blocks, internal standard) were weighed into the I-Chem vials under air using a Mettler-Toledo Quantos automated powder-dispensing analytical balance with acceptable tolerance set to ±2% by mass. Liquid chemicals (a few of the halide building blocks) were added via microliter pipette. Reaction vials were loaded onto the synthesis machine and the automated synthesis procedures were executed. Generally, this entailed atmosphere exchange of the reaction vials using an automated Schlenk-line procedure (vacuum applied for 5 min. followed by 1 min. backfill with argon, 10 6-min cycles, for a total time of 1 h to degas 36 reaction vials), followed by solvent addition (pre-programmed selection per vial of up to 20 different solvents) via automated syringe pump, heating and stirring for the given reaction time, and cooling. Afterwards, the reactions were either purified by preparative HPLC for molecular characterization and LCMS response factor curve generation or were quantified by direct injection into LCMS.

Reaction Data Analysis. Reaction data sets shown in main text figures were visualized and analyzed using GraphPad Prism 9.3.1. Statistical comparisons of general reaction conditions utilized repeated measures one-way ANOVA with the Geisser-Greenhouse correction and Šídák's multiple comparisons test.

Code and Data Availability. All data and code generated as part of this study are freely accessible either in the supplementary material or in open repositories. Code for the simulation example, model selection, and substrates clusterization, as well machine- and human-readable versions of the reaction data are freely accessible at Zenodo. The automated synthesis code, machine parts list and build guide, datamined commercially available building block set, checklist for reporting and evaluating machine learning models, and tabulated numerical data underlying FIGS. 2-4 are deposited at Zenodo. The code used in the building block selection process is available at Zenodo. All code related to the closed-loop optimization is freely available at Zenodo.

### Example 2—Automated Synthesis Machine

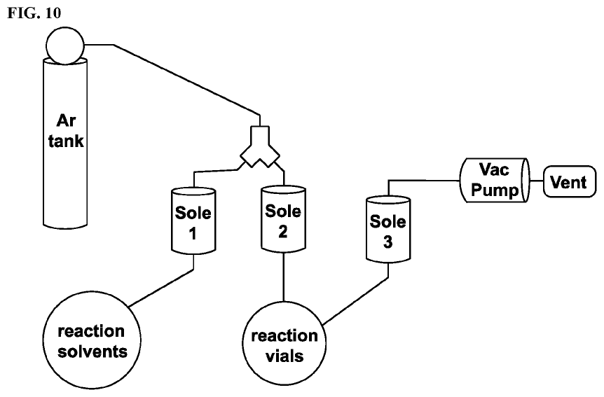

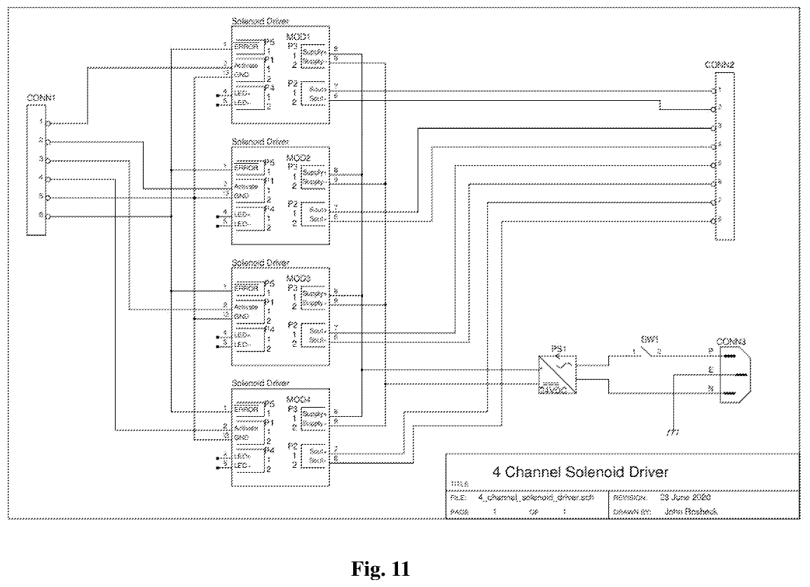

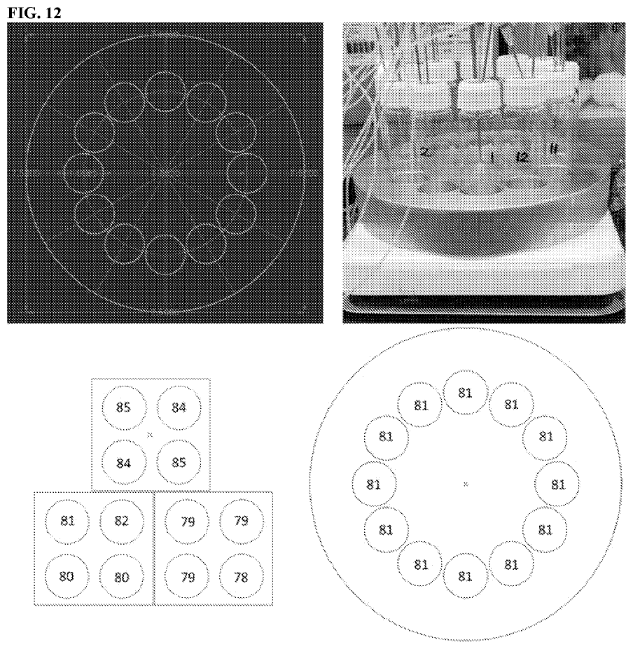

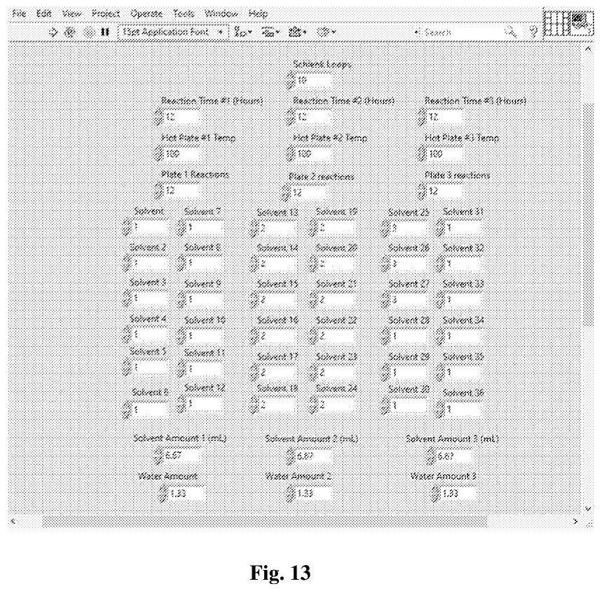

Description of the Automated Synthesis Machine. The automated synthesis machine was constructed using the same reconfigurable valves and syringe pumps as our prior reported iterative small molecule synthesizer in a unique configuration suited for parallel reaction testing and driven by custom Lab VIEW software. Each reaction vial was connected to the solvent reservoirs via syringe needle, PEEK tubing, and Luer lock fittings, through two computer-controlled 9-port 4-valve modules and 10 mL computer-controlled syringe pump (FIG. 9). Solvents were stored in Pyrex glass media bottles and connected via PEEK tubing, Luer lock fittings, and a 3-port Cole-Parmer VapLock solvent delivery cap to both the argon manifold and the valve modules. The third port on the solvent caps was sealed with a Luer plug fitting which was temporarily removed when solvents were refilled on the system to allow for argon sparging. Each reaction vial was also connected to the argon/vacuum manifold via a syringe needle, PEEK tubing, and Luer lock fittings, through passive 9-port valves and solenoid valves (FIG. 10). The argon cylinder was maintained at ˜5 PSI regulator setting and did not need to be replaced for the entirety of this study. Solenoid valves were controlled via a National Instruments USB-6001 Multifunction I/O Device and powered by a custom 4 Channel Solenoid Driver from the University of Illinois Electronic Services (FIG. 11). Similar solenoid valve drivers are commercially available. The three computer-controlled hot plates each had a circular heating block with 12 positions for reaction vials evenly spaced in a circle from the centrally located heating probe. This heating block effectively eliminated any “edge effects” associated with typical square or rectangular heating blocks which can cause stirring and heating differences dependent on reaction position via differing relative distance from the heating probe (heating) and magnetic field (stirring) (FIG. 12). The height of the heating block was also configurable: it could be increased by stacking aluminum heating blocks or decreased by filling equal quantities of sand into each reaction position. For the automated syntheses in this work, the heating block was as shown in FIG. 8, and heated to the height of the 8 mL reaction volume in each reaction vial. This enabled reaction solvents to be heated to reflux without redistribution throughout the manifold or between reaction vials as the remainder of each vial exposed to ambient air acted as air-cooled reflux condensers. The custom Lab VIEW software was designed as one general code that was executed with controllable parameters of: number of parallel reactions (between 1-36 reactions), solvent selection per reaction position (between 1-20 solvents), organic solvent quantity (mL), water quantity (mL, for varying the ratio of organic solvent:water), reaction temperature (° C.), reaction time (hours), and stirring rate (rpm) (FIG. 13). Users of the automated synthesizer load relevant chemicals onto the synthesizer, select the reaction parameters from the ‘front panel’ of the Lab VIEW code, and run the program. Then, the code asks the user to ensure the equipment is online and that there is sufficient solvent in the reservoirs: when the user clicks “OK” on this prompt, the automated code executes the full synthesis. A full parts list needed to build this synthesis machine is available in Table 1.

| | | | | | | Parallel | |
|---|---|---|---|---|---|---|---|
| Description | Part number | Vendor | Price | Quantity | Total | capability | 36 |
| Syringe Pump | KEMV6 | J-KEM | $1,742.48 | 1 | 1,742.48 | | |
| | Pump | | | | | | |
| 10 ml glass syringe | SPGS-10000 | J-KEM | $ 165.00 | 1 | 165.00 | | |
| eight port distrubution valve | SPDV-8 | J-KEM | $265.10 | 1 | 265.10 | | |
| quad stack distribution module | Selection-4/8 | J-KEM | $4,489.60 | 2 | $ 8,979.20 | | |
| J-KEM Shipping to UIUC | Shipping | J-KEM | $321.80 | 1 | $ 321.80 | | |
| Female to Female PEEK luer | P-658 | IDEX Health & Science, LLC | $ 13.31 | 1 | $ 13.31 | | |
| Female to Male Leur | P-675 | IDEX Health & Science, LLC | $ 9.74 | 36 | $ 350.64 | | |
| PEEK Y assembly | P-513 | IDEX Health & Science, LLC | $ 33.21 | 3 | $ 99.63 | | |
| PEEK 9 port manifold | P-191 | IDEX Health & Science, LLC | $ 136.35 | 7 | $ 954.45 | | |
| plug | P-316 | IDEX Health & Science, LLC | $ 2.12 | 20 | $ 42.40 | | |
| flangeless ferrule | P-200 | IDEX Health & Science, LLC | $ 1.35 | 300 | $ 405.00 | | |
| flangeless male nut | P-202 | IDEX Health & Science, LLC | $ 1.35 | 160 | $ 216.00 | | |
| 100 ft 1/16″ .03″ FEP tubing | 1520 | IDEX Health & Science, LLC | $ 165.13 | 5 | $ 825.65 | | |
| 100 ft 1/8″ .062″ FEP tubing | 1521 | IDEX Health & Science, LLC | $ 248.68 | 1 | $ 248.68 | | |
| IDEX Shipping to UIUC | Shipping | IDEX Health & Science, LLC | $ 50.00 | 1 | $ 50.00 | | |
| C-MAG HS 7 CONTROL S001 | 0010003303 | IKA Works, Inc | $ 867.75 | 3 | $ 2,603.25 | | |
| IKA Shipping to UIUC | Shipping | IKA Works, Inc | $ 75.00 | 1 | $ 75.00 | | |
| Solenoid Valves | MTV-2K- | Takasago Electric, Inc | $ 56.44 | 4 | $ 225.76 | | |
| | N1/4UGH | | | | | | |
| 100 5 ml plastic syringe | 4050-000VZ | W. W. Grainger Inc | $ 20.83 | 1 | 20.83 | UIUC Shops | |
| | | | | | | Breakdown | |
| 3 pk rare earth stir bars | z671665 | Sigma-Aldrich inc | $ 17.10 | 10 | $ 171.00 | Parts Cost | Labor Cost |
| Aluminium heating blocks | na | UIUC SCS machine shop | | | $ 1,185.69 | $ 287.75 | $ 897.94 |
| Solenoid Valve Driver | na | UIUC SCS electronics shop | | | $ 258.80 | $ 82.80 | $ 196.00 |
| 8 AI (14-Bit, 20 kS/s), | USB-6001 | NATIONAL INSTRUMENTS | $ 194.00 | 1 | $ 194.00 | | |
| 2AO (5 kS/s/ch), 13 DIO | | COR | | | | | |
| USB Multifunction | | | | | | | |
| I/O Device | | | | | | | |
| Straight Adapters, Tube | 1 | McMaster-Carr | $15.82 | 1 | $ 15.82 | | |
| Stem, Tube | | | | | | | |
| Straight Adapters, Tube Female | 1 | McMaster-Carr | $16.00 | 1 | $ 16.00 | | |
| Threaded Pipe | | | | | | | |
| I-chem clear clean snap vials | SS236-0040 | Fisher Scientific | $135.20 | 2 | $ 270.40 | | |
| with 0.125 in Septa | | | | | | | |
| Fisherbrand ™ Multistage Gas | 10-575-135 | Fisher Scientific | $ 195.37 | 1 | $ 195.37 | | |
| Cylinder Regulator | | | | | | | |
| KNF Neuberger | 13-878-25 | Fisher Scientific | $ 1,542.33 | 1 | 1,542.33 | | |
| Vacuum Diaphragm | | | | | | | |
| Pump, Model: UN810.3FTP | | | | | | | |
| | | | Total = | | $ 19,911.26 | | |

### Example 3—Platform Validation

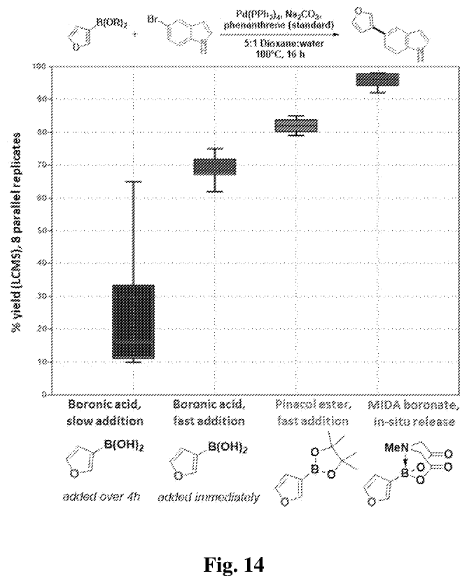

To validate the platform's performance and reproducibility in the context of heteroaryl cross-coupling, we investigated its performance on the synthesis of furanylindole 1 with the read-out being reaction yield. We first investigated the role of the boron species and discovered that the in-situ release of the MIDA boronate yields the highest overall levels of reproducibility and yield when compared to boronic acids and esters (FIG. 14). This test reaction featured the most statistically common palladium, base, solvent, and temperature in the heteroaryl cross-coupling literature (Reaxys database, reference. This result suggests that both the stability and the reactivity of the boron species and halide is critical for a reproducibly high yield. In the case of the slow addition of the boronic acid, the relative concentration of the boronic acid in the reaction mixture is kept low and it is prevented from decomposing by being added slowly to the reaction: the resulting great variation in outcome suggests the halide coupling partner may be decomposing during that slow addition time. Fast addition of the boronic acid leads to higher yield and a more reproducible outcome, however the yield is diminished potentially due to decomposition of the high-concentration, labile boronic acid over time. Use of the pinacol boronic ester prevents decomposition of the boron species and raises the yield and reproducibility, but it caps out at near 80% yield potentially due to the slower reaction kinetics of the more hindered pinacol ester allowing for degradation of the halide over time. The in-situ hydrolysis kinetics of the MIDA boronate appears commensurate to ensuring a high yielding and highly reproducible automated reaction. While we did not measure the hydrolysis kinetics in this study, our prior study of 2-furanyl MIDA boronate's hydrolysis kinetics under similar conditions suggests it is likely converted to the boronic acid over a period of one hour.

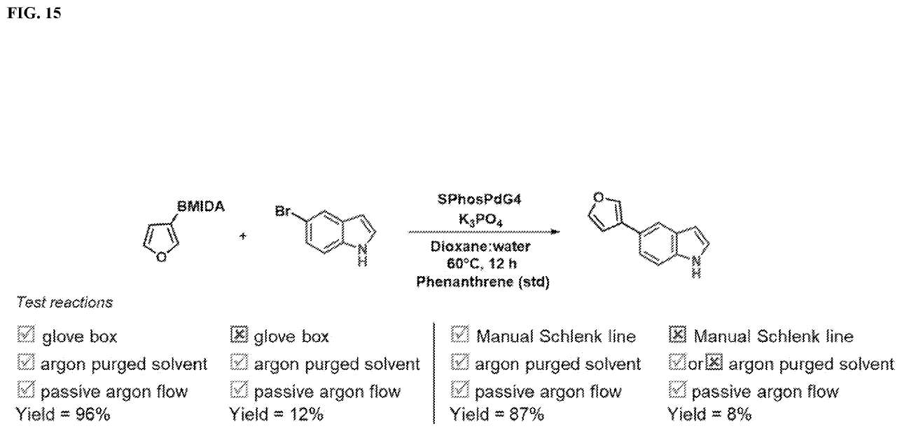

While our prior use of Pd(PPh₃)₄ necessitated a glovebox due to air sensitivity of that catalyst, we were surprised to discover the use of air-stable palladium precatalysts (e.g. SPhos Pd G4) weighed in air led to diminished yields despite the passive argon flow on our automated synthesizer. We manually performed a series of head-to-head control experiments (FIG. 15) to probe the environmental controls necessary to prevent oxygen-mediated diminished yields. From these experiments, it became clear that the residual air in the headspace of the reaction vials was the contributing factor for the diminished yields and that passive argon purging was not effective at replacing the atmosphere. We also noted that manual Schlenk line vacuum/backfill cycles replicated similar performance to reactions set up entirely in a glovebox. This new understanding afforded the options of either manually subjecting reaction vials to Schlenk line procedures prior to introduction to the synthesis machine, or to automate the process after introduction onto the instrument. We designed a simple Schlenk line incorporated onto the machine consisting of solenoid valves separating the argon cylinder and the vacuum pump (see FIG. 10). We measured the time which was required for the vacuum to reach its maximum vacuum while connected to the 36-reaction gas manifold via audio recording (˜4 min., the vacuum pump is quietest when it is at maximum vacuum) and tested 5-minute vacuum followed by 1-minute argon backfilling cycles for their effectiveness in promoting the heteroaryl cross-coupling (FIG. 16). From this data it is apparent that there is a direct correlation between the number of automated Schlenk cycles and the corresponding reaction yield, exceeding that of the manual Schlenk cycles and becoming statistically indistinguishable in yield from the glovebox using reaction at ≥9 cycles. For the remainder of this work, we utilized 10 Schlenk cycles as a conservative approach which only required one hour of automation time. The design and optimization of this instrument led to reproducibility of ±2% yield between replicates throughout the optimization.

### Example 4—Problem Definition and AI Framework for the Optimization of Reaction Generality

The goal of this study is to discover general sets of reaction conditions for classes of small molecules, specifically heteroaryl-(hetero)aryl Suzuki-Miyaura cross-coupling, where at least one coupling partner is a heteroaryl ring. Heteroaryl cross-coupling is a problem of high impact as the motif is highly represented in the areas of materials science, natural products, and drug molecules. While there is a huge structural diversity of heteroaryl Suzuki coupling in the literature, there is surprising lack of generality for the reaction conditions. Scientists in this subfield usually test a small set of conditions based on what they own or have experience with regarding catalyst, base, solvent, ligand, and temperature. Problematically, only the highest yielding condition per pair of substrates is typically reported. Conversely, studies regarding reaction optimization report a wide range of reaction conditions for a very limited scope of substrates. To solve this problem generally for a broad class of molecules requires a diverse and large set of representative substrate pairs to be tested under many general reaction conditions. The reaction conditions with the highest average yield across a representative range of substrates will be considered the most general. Optimizing for generality in this manner is new.

To formulate the problem mathematically, let us denote a set of possible reaction conditions as C={c}, a set of substrate pairs as S={s} and a reaction yield as a function of substrates and reaction conditions, y(s, c). Then, the objective function we aim to maximize, f(c), is given by:

$$f ( c ) = \frac{1}{\vert S \vert} {\sum }_{s ∈ S} y ( s , c ) \qquad ( 1 )$$

Then, the general conditions c_{general} are given as

$${c}_{general} = {arg max}_{c ∈ C} f ( c ) \qquad ( 2 )$$

At first glance, the problem of identifying c_{general} in the least number of experiments resembles standard Bayesian optimization (BO). However, there is a substantial difference: in all BO algorithms, each experiment/measurement performed, immediately provides information about the objective function one wishes to optimize. In contrast, experimental evaluation of f(c) in our problem requires multiple experiments (because summation in eq. 1 runs over the entire set S)—that is, determination of f(c) for given conditions requires experiments with every pair of substrates in the S set. In order to address this problem, we modify the standard BO approach by constructing a surrogate model for predicting reaction yields, ÿ(s, C), and then use its predictions to estimate \hat{f}(c) according to equation (1) using model's prediction for the yet-unperformed reactions (Note: The difference is that in standard BO we would have observed f(c) for the “seen” conditions and estimated for the “unseen” ones; in our case f(c) is estimated even for the already tested conditions, unless entire S had already been tested). Based on these considerations, the optimization over C (selection of the next conditions to examine) is performed with standard Bayesian optimization techniques, whereas sampling of S is achieved using an active learning approach. In particular, we decided to choose substrate pairs based on the model's prediction uncertainty for given substrates under given reaction conditions: the highly uncertain (low confidence) predictions indicate missing information, and providing the model with the corresponding experimental data should decrease its uncertainty the most.

In selecting a suitable model, we considered two factors: a) uncertainty estimation and b) estimated performance in the above scheme. Regarding (a), we sought a model offering prediction uncertainty commensurate with prediction error—for instance, highly confident predictions with high error are undesirable. Regarding (b), we wished to find means of testing various possible models before actually commencing our own closed-loop optimization. These aspects are elaborated on in the following subsections.

Benchmarking Prediction Uncertainties. We first decided to evaluate uncertainty in predicting reaction yield of various models using a previously published dataset of 5,760 Suzuki coupling reactions. This set was chosen because (i) trivially, the reactions were directly related to our current study, and (ii) because they were performed in a highly standardized fashion and automatically (in a flow reactor), and the reported yields could thus be expected to be free from random errors. Each reaction in the set was performed under ˜300 different conditions differing in the solvent, ligand and base used. On the downside, the substrates were based on only two structural motifs, quinoline and indazole, differing in the type of boronic moiety (boronic acid, trifluoroborate salt, boronic acid pinacol ester) and the coupling partners (iodide, bromide, chloride, triflate). This means that this dataset was not suitable to train models to suggest the “next” substrates. For each combination of structural motifs, we decided to include only one type of a halide and boronic functionality in order to avoid redundancy. This left 588 reactions that were used to examine different uncertainty estimation techniques.

The models we considered were all based on neural networks, NNs. There are several different approaches to determine uncertainty of NN prediction. We tested the following procedures: i) traditional ensembling, ii) bootstrap ensembling, iii) Monte Carlo dropout, iv) Bayesian neural network with flipout layers, v) NN with direct uncertainty estimation using negative log likelihood loss (NLL), and vi) a combination of a neural network with Gaussian Process (GP). The last tested method (vi) uses GP as a model of statistical inference in which NN part can either estimate (1) the mean value of the function (GP then estimates the uncertainty), (2) the covariance between different points (how the uncertainty changes with the distance from the observed points), or (3) both. Inclusion of GP supplemented with a NN component appeared particularly appealing, since in the absence of meaningful metric quantifying difference between reaction conditions, we could learn it from the data by jointly training GP with a NN kernel (similar approaches were recently used in Bayesian optimizations.)

NN models were implemented using Keras and Tensorflow with GPflow for modelling Gaussian Processes. Methods (i)-(v) of uncertainty estimation were tested on two feed-forward neural networks with two hidden layers. The first network had two dropout layers whereas the second had no dropout regularization. For each network, architecture hyperparameters were first optimized using hyperas. In contrast, the neural components of GP models, (vi), were subjected to grid search over a range of architectures (illustrated in FIG. S12) defined by the following parameters: 1) dimension of the embedding layer Nₑ (either 10, 50, 100 or 200 neurons), 2) dimension of the next hidden layers Nh (50, 100, 150 or 200 neurons with ReLU activation), 3) number of hidden layers L_{H} (1, 2 or 3), 4) dropout probability D (taken from range 0.1-0.9 with 0.1 interval), 5) dimension of the output layer No (1, 10, 50, 100, 150, 200 neurons with ReLU activation) as well as 6) L2 regularization weight (either 0.001, 0.01 or 0.05). The kernel function built on top of this embedding component was chosen as either Radial basis function, Matern32 or Matern52.

For every AI model considered here, we use the same input representation of reaction, which is a concatenation of the following vectors: 1) FCFP6 fingerprint representation of bromide coupling partner, 2) FCFP6 fingerprint representation of BMIDA coupling partner, 3) scalar representing temperature, 4) one-hot encoding of solvent, 5) one-hot encoding of base and 6) one-hot encoding of ligand. Regarding fingerprints, we represent them in a “dense format” prepared as follows. First, we collect all identifiers (numbers representing specific substructures in a fingerprint vector present in all substrates (e.g., bromides) within our scope, counting the number of occurrences of each identifier. Next, we sort these identifiers according to their frequency within the dataset, and assign each one to a component of a “dense” fingerprint vector (whose length thus corresponds to the total number of identifiers). Finally, for a given molecule, we set the values of the vector's components with the counts of corresponding identifiers in the molecule's FCFP6 fingerprint. Note that with such representation, the resulting uncertainty in the AI model does depend not only on the similarity between the substrates in training/test sets but conditions as well-including possible non-additive effects (e.g., that the same change of conditions may influence substrate pair A more than substrate pair B). In addition, our model before training is totally agnostic about the similarity between reaction conditions (i.e., solvents)-everything is to be inferred (learned) directly from the data.

As the prediction uncertainty is going to provide the basis for our acquisition algorithms—in particular, it is expected to indicate model's “lack of knowledge”—we analysed the distributions of uncertainty produced by different models. In doing so, we were interested in models whose prediction uncertainty changes monotonically with the prediction error—that is, those in which datapoints with higher prediction error have higher uncertainty than datapoints with lower prediction error (prediction of relative values).

With this in mind, we compared, for both training and test sets, the mean absolute error and mean prediction uncertainty. Specifically, we evaluated our criterion by examining two differences: difference between average errors ΔMAE-MAEₜₑₛₜ−MAEₜᵣₐᵢₙ and difference between average uncertainties ΔU=Uₜₑₛₜ−Uₜᵣₐᵢₙ. We required that both ΔMAE and ΔU a) have the same sign and b) have absolute magnitude greater than 1% (which is true for all ΔMAE in our comparisons). In all experiments, a common, randomly selected 20% of the entire dataset was used as the training set whereas the remaining 80% served as the test set—such division was intended to mimic initial stages of closed-loop optimization, when the model has the knowledge of only a small fraction of the total space.

Among all trained NN models (Table 2), only NLL and flipout fail to fulfil our criterion, as the average uncertainties on training and test sets are virtually the same, whereas the test MAE is, in general, significantly higher than the training one. Among the remaining non-GP models, the best results come from the ensemble approaches. However, since their performance is comparable, we decided to make additional test with even smaller training set, 5% of the entire dataset (Table 3). In this test, both bootstrap and traditional ensemble models passed our criterion, with the latter providing lower MAEₜₑₛₜ.

Regarding the GP-based models, they all fulfil our criterion (Table 2), with a GP with NN-based kernel providing the lower test error (24.7%). Although the value is considerably higher than for the “pure” NN models (15.2% for traditional ensemble mentioned above), the training MAE for GP(NN), 7.3%, is lower than that for NNE, 8.2%. By definition—as GP computes the probability distribution given observed datapoints—the combined model is guaranteed to converge to the ground truth with the expansion of the dataset. Therefore, we select GP with NN kernel for further tests alongside with the traditional ensemble of neural networks.

TABLE 2
Average errors and prediction uncertainties for NN-based models with
train:test ratio of 20:80.

| | training set | | test set | | | | |
|---|---|---|---|---|---|---|---|
| name | MAEₜᵣₐᵢₙ | Uₜᵣₐᵢₙ | MAEₜₑₛₜ | Uₜₑₛₜ | ΔMAE | ΔU | criterion |
| bootstrap ensemble | 0.084 | 0.109 | 0.152 | 0.126 | 0.068 | 0.017 | TRUE |
| traditional ensemble | 0.082 | 0.11 | 0.152 | 0.124 | 0.07 | 0.014 | TRUE |
| MC dropout | 0.12 | 0.09 | 0.16 | 0.1 | 0.04 | 0.01 | TRUE |
| NLL | 0.077 | 0.012 | 0.166 | 0.011 | 0.089 | −0.001 | FALSE |
| flipout | 0.166 | 0.102 | 0.185 | 0.103 | 0.019 | 0.001 | FALSE |
| GP(NN mean & | 0.063 | 0.053 | 0.256 | 0.217 | 0.193 | 0.164 | TRUE |
| kernel) | | | | | | | |
| GP(NN kernel) | 0.073 | 0.045 | 0.247 | 0.158 | 0.174 | 0.113 | TRUE |
| GP(NN mean) | 0.119 | 0.025 | 0.250 | 0.054 | 0.131 | 0.029 | TRUE |

MAEₜᵣₐᵢₙ, Uₜᵣₐᵢₙ-mean absolute error and average uncertainty on the training set,
MAEₜₑₛₜ, Uₜₑₛₜ-mean absolute error and average uncertainty on the training set,
ΔMAE = MAEₜₑₛₜ-MAEₜᵣₐᵢₙ, ΔU = Uₜₑₛₜ-Uₜᵣₐᵢₙ.
We expect both differences to fulfil the criterion of a) having the same sign and b) having absolute magnitude >1%.

TABLE 3
Average errors and prediction uncertainties for
NN-based models with train:test ratio of 5.95.

| | training set | | test set | | | | |
|---|---|---|---|---|---|---|---|
| name | MAEₜᵣₐᵢₙ | Uₜᵣₐᵢₙ | MAEₜₑₛₜ | Uₜₑₛₜ | ΔMAE | ΔU | criterion |
| traditional | 0.06 | 0.05 | 0.176 | 0.07 | 0.116 | 0.02 | TRUE |
| ensemble | | | | | | | |
| bootstrap | 0.08 | 0.13 | 0.195 | 0.15 | 0.115 | 0.02 | TRUE |
| ensemble | | | | | | | |
| flipout | 0.11 | 0.11 | 0.197 | 0.12 | 0.087 | 0.01 | FALSE |
| MC | 0.11 | 0.11 | 0.207 | 0.12 | 0.097 | 0.01 | FALSE |
| dropout | | | | | | | |
| NLL | 0.15 | 0.17 | 0.214 | 0.19 | 0.064 | 0.02 | TRUE |

MAEₜᵣₐᵢₙ, Uₜᵣₐᵢₙ-mean absolute error and average uncertainty on the training set,
MAEₜₑₛₜ, Uₜₑₛₜ-mean absolute error and average uncertainty on the training set,
ΔMAE = MAEₜₑₛₜ-MAEₜᵣₐᵢₙ, ΔU = Uₜₑₛₜ-Uₜᵣₐᵢₙ.
We expect both differences to fulfil the criterion of a) having the same sign and b) having absolute magnitude >1%.

Above, we performed model selection based on criteria aiming to reflect, on average, the relationship between the model's uncertainty and prediction error—to put it more intuitively, whether the model is able to ‘predict’ the degree of its own error. In this experiment, we focused on the two aspects of the correlation between model's error and predicted uncertainty: qualitative agreement reflected by the Kendall t coefficient and quantitative agreement measured with the z-score

$$\frac{\vert {y}_{exp} - {y}_{pred} \vert}{{σ}_{pred}} .$$

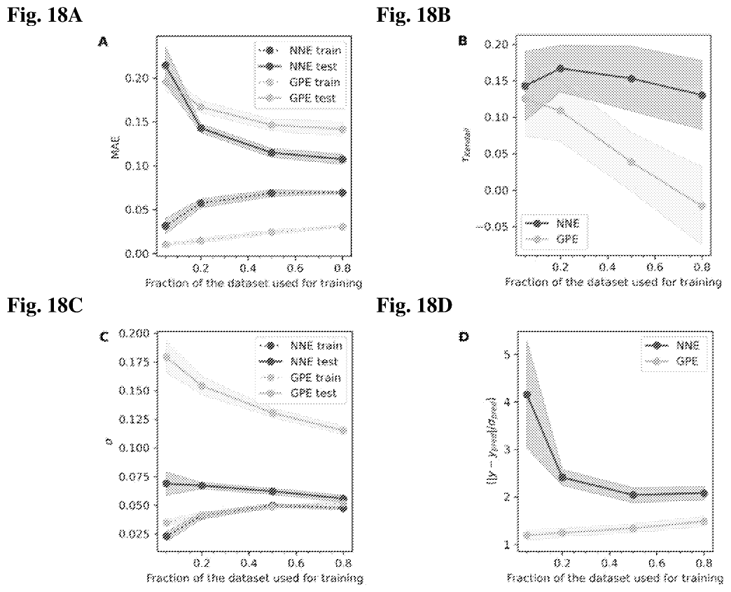

In FIG. 18B, one can see that both models show significant qualitative correlation between errors and uncertainties in sparse-data limit, mimicking initial part of the optimization loop. However, in the case of GPE, this correlation tends to decrease as the dataset size increases. This is accompanied by the decrease of both model's error on the test set (FIG. 18A) as well as its uncertainty (FIG. 18C). Quantitatively, GPE's uncertainty better approximates prediction error as the corresponding z-score is significantly lower than in the case of NNE (FIG. 18D). Finally, GPE exhibits much lower training error than NNE (which probably arises directly from the GPE's definition). This last feature is likely the reason why GPE ultimately outperformed NNE in the calibration simulation-despite making better predictions on the test set, the higher training error of NNE limits its ability to correctly order conditions when trained on almost completed reaction space.

### Selection Algorithm for Closed-Loop Experiments.

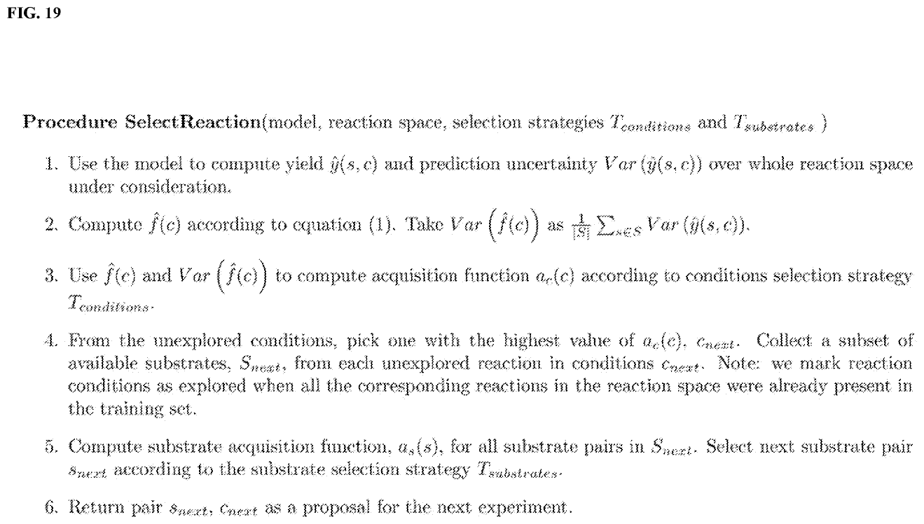

Next, we considered selection of the proposed experiments given the trained model, the reaction space N_{substrate pairs}×N_{conditions} (with indication which reactions had already been performed), and selection strategies, T_{conditions} and T_{substrates}. The overall protocol is outlined in FIG. 19. In short, we 1) use the model to predict yields along with the prediction uncertainty over the entire reaction space (i.e., space of all not-yet-performed reactions); 2) transform this result into the objective function; and 3) select substrate pair and reaction conditions to test in the next experiment according to the selection strategies. In general, we expect the selection strategy to returns a “score” for each candidate, with the best candidate indicated by the highest score. Regarding T_{conditions}, it is expected to take the estimate of our objective f(c) and return a score quantified by the conditions' acquisition function ac (c). In the tests described, we will examine two well-established BO selection strategies: a) probability of improvement (PI)—that is, probability that the new conditions c_{new} will be better than the best of those already seen; and b) expected improvement (EI)—expected value of improvement over the-best-so-far conditions, computed over probability distribution at c_{new}. Both (a) and (b) are based on the assumption that at each c, the distribution of the objective function is normal, with mean given by f(c) and standard deviation given by the uncertainty of f(c). Regarding T_{substrates}, it is supposed to score substrates yet unexplored under given conditions. In particular, we consider two such strategies: i) random selection and ii) selection by maximum uncertainty (discussed in the beginning of Section 3).

### Simulation of Closed-Loop Optimization for General Conditions.

Next, anticipating our own experiments, we simulated post-hoc a “closed-loop” optimization using experimental data published previously. Since the dataset we used for uncertainty calibration involves only two different structural motifs for each of the coupling partners, it could not be used to investigate substrate selection strategies. To our best knowledge, there is no publicly available dataset of Suzuki reactions performed in a standardized manner and covering a broader scope of substrates. Therefore, we decided to search for other Pd-catalyzed reactions and chose a dataset reported by Santanilla et al for C—N coupling (Buchwald-Hartwig amination). The new dataset comprises 1536 robot-performed reactions and covers 48 different conditions for each of 32 different substrate pairs.

The simulations of the closed-loop protocol were then performed as follows. First, we randomly selected 5% of the entire data as the initial training set and used it to train different models for prediction of yields y(s,c) (these predictions were then to be used to compute f(c)). Next, the closed-loop iterations were performed until the entire dataset was explored. During each iteration, we (i) trained a given model on the already-seen data; (ii) selected the “next pair of substrates according to the procedure in FIG. 19; and (iii) extended the training set by the inclusion of the proposed reaction. To account for the random component of this procedure (stemming from the different choices of the initial set as well as from the random initialization of NN parameters), we performed 100 independent simulations for each model to collect statistics.

### Performance Metrics.

We used two metrics to describe the quality of prediction: rank of the best conditions and yield of conditions predicted to be the best. The first metric designates the position of the best conditions (actual, experimental) in the ranking produced according to the model's predictions, e.g., value 0 means the model correctly predicts particular conditions as being better than any others. The second metric indicates the experimental average yield of conditions predicted to be the best. In other words, the first measure describes how good a model is in finding the best conditions, whereas the second describes how good are the estimates of the corresponding average yield.

### The Closed-Loop Simulations.

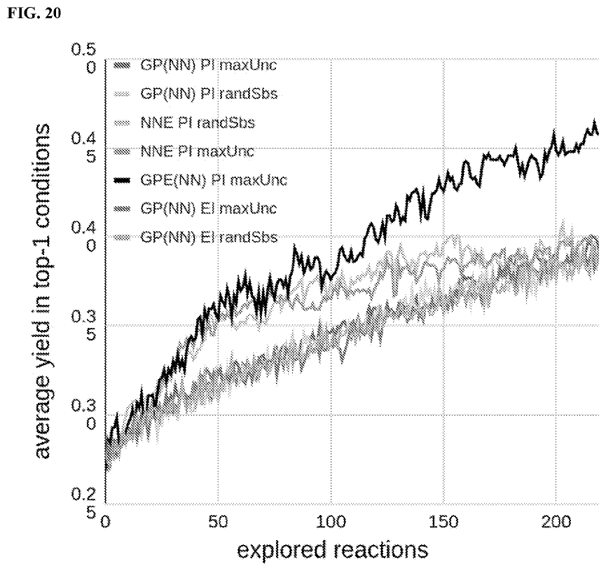

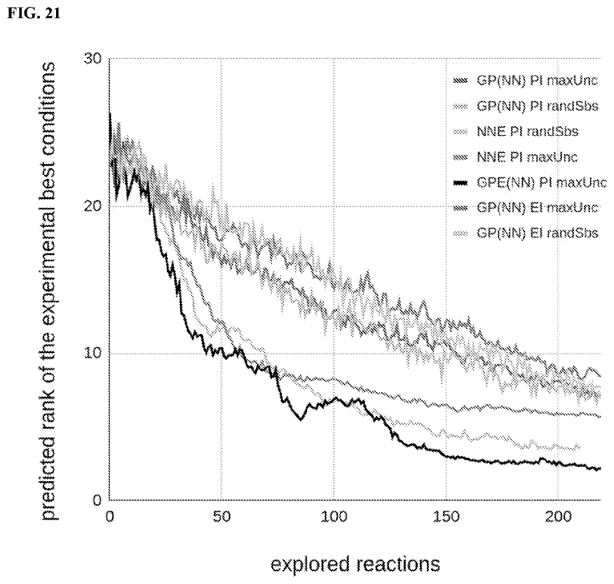

In the following experiments, we focus on two models from the previous section: Gaussian Process with a Neural Network kernel (henceforth denoted as GP(NN)), and a Neural Network Ensemble (NNE). Note that the ensemble type used here refers to the “traditional” ensemble without dropout regularization (as opposed to “bootstrap” ensemble), i.e., each model in the ensemble was trained on the same data but with different values of initial parameters. The combinations of the following selection strategies were tested: expected improvement (EI) and probability of improvement (PI) for T_{conditions}, whereas for T_{substrates} we used either random strategy (randSbs) or selection of substrates with the highest prediction uncertainty (maxUnc). In the GP(NN) model, all four possible combinations lead to similar model performance (FIGS. 20 and 21).

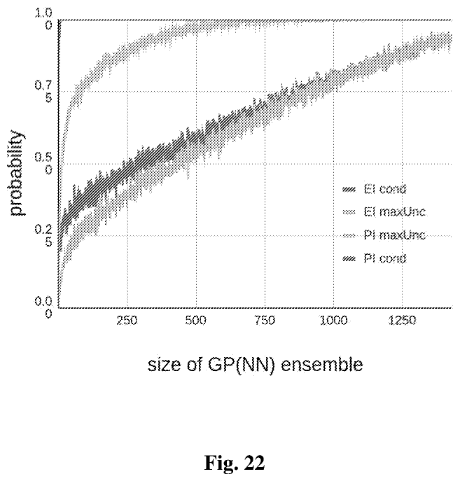

Comparison of GP(NN) and NNE indicates that NNE outperforms GP(NN) in both ranking conditions and in proposing conditions offering high average yield during initial ˜150 iterations. Subsequently, both methods ultimately converge to comparable results.
Relatively poor performance of GP(NN) can be attributed to the neural network used as a kernel. In the current implementation, it is not possible to use stochastic gradient descent during training (the gradient and update of the parameters are computed on the whole training set at once), which effectively causes the NN component to get stuck in the nearest local minimum whose location depends on the initial values of parameters. In order to overcome this limitation, we decided to make an ensemble of GP(NN)—abbreviated as GPE(NN) (black curves in FIGS. 20 and 21)—which turned out to outperform NNE (similar modifications are already known in the literature. With these improvements in hand, we moved on to the optimization of the ensemble's size and acquisition function type. We note that extensive (brute-force) testing of GPE(NN) models of different sizes is rather costly, therefore we decided to train a large GPE(NN) model consisting of 2000 independent GP(NN) models and record all intermediate results. Then, the result of a smaller GPE(NN) could be simulated by choosing from the recorded results a subset of a desired size. For each size in the range from 2 to 1200, we randomly selected 500 such samples and used such a population to compute probabilities that smaller GPE(NN) s would make the same choice as the reference 2000-model GPE(NN) in the very first step of a closed-loop simulation (FIG. 22). For PI acquisition function, an ensemble as small as 10 models was able to select the same conditions as the reference with probability above 0.98, whereas for PI+maxUnc selection strategy, an ensemble composed of 100 models could predict the same conditions and substrates with probability ˜0.8. In the case of EI, probability of selecting the same substrates and conditions (as the reference, large ensemble) converges very slowly, even for a 1000-membered ensemble, the corresponding probabilities are about 0.78-0.79. Consequently, we choose the PI acquisition function for further study and concluded that an ensemble of 100 models is a reasonable compromise between computation time and accuracy.

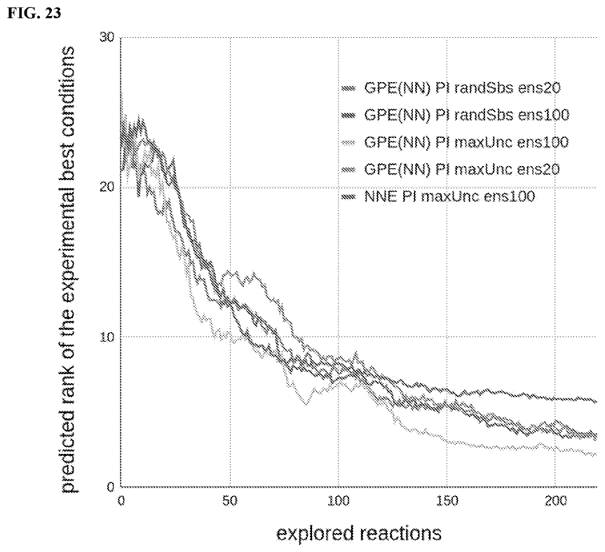

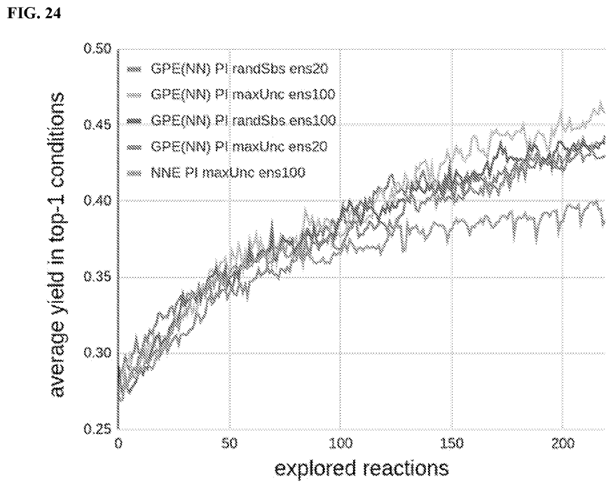

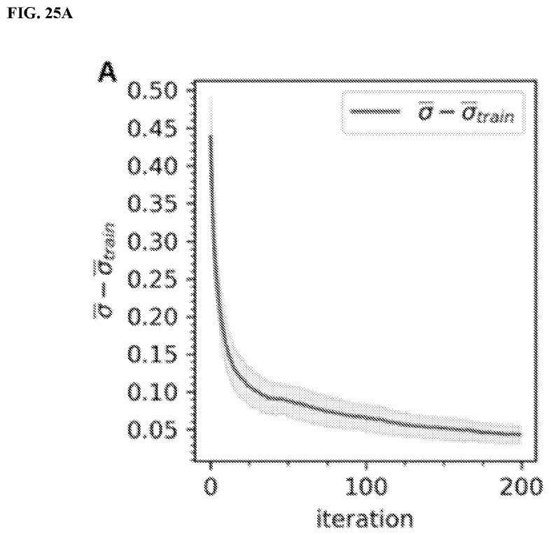

Next, we compared GPE(NN) with 20 and 100 models with PI+random and PI+maxUnc selection strategies (FIGS. 23 and 24). For the ensembles consisting of 20 GPE(NN), maxUnc gives slightly worse results than random substrate selection but finally (step 150 and further) converges into virtually the same results. In the case of a 100-membered ensemble, substrate selection based on uncertainty gave slightly better results whereas random substrate selection gave the same results as with the smaller ensemble. These results suggest that maxUnc substrate selection procedure outperforms random selection only for a sufficiently large NN ensemble.

### Simulation of the Stop Criterion.

In this section, we examine the uncertainty-based stop criterion. In particular, we monitor the difference between average uncertainty on the whole search space, σ̅, and the average uncertainty of the training set, σ̅ₜᵣₐᵢₙ. If one considers the average uncertainties as a measure of entropy (lack of information), such a difference could be interpreted as the remaining information one can infer from the experiments. Intuitively, this “information gap” should decrease as the closed-loop experiments proceed, reaching zero in the limit when the entire search space is examined—such behavior is indeed observed on our calibration dataset (FIG. 25A). Furthermore, if the low information gap truly indicates that the model “probably learned most of what there was to learn,” then models with lower information gaps should tend to provide ‘good’ predictions about the general conditions. In order to verify this hypothesis, (1) we consider a range of thresholds for the information gap; (2) for each threshold value, we stop each of the simulated replicas as soon as the model's information gap reaches the threshold; and (3) we compute the fraction of replicas for which the true best reaction conditions were amongst the top-k predictions. We then use the result to estimate the probability that the model places the true general conditions within its top-k predictions using Laplace's rule of succession (if n models reached the information gap threshold and s of them places true general conditions in top-k predictions, then the estimated probability is

$$\frac{s + 1}{n + 2} ) .$$

The variance of the corresponding Beta distribution serves as an uncertainty of this estimation. As can be seen in FIG. 25B, the top-5 and top-3 probabilities increase as the information gap goes to zero, indicating that ending the optimization at a low value of ‘information gap’ (here the threshold was about 2.3%) will provide a proper optimum.

### Example 5—Building Block Datamining, Clustering, and Reaction Scope Selection Substrate Scope Building Block Datamining

Defining generality in the context of heteroaryl Suzuki coupling required prospectively determining a substrate scope with maximum representation of accessible heteroaryl chemical space. To do so required the creation of a large in-silico molecular LEGO-kit consisting of all commercially available (hetero)aryl halides and (hetero)aryl MIDA boronates. To datamine this library, we used a web and database scraper to find purchasable building block molecules available from common chemical suppliers. This scraper catalogued every aryl halide, heteroaryl halide, and MIDA boronate reported in the PubChem database and then scraped and cross-referenced those structures to real pricing data from the back- and front-ends of the world's largest and most reliable fine chemical suppliers (Sigma-Aldrich, Oakwood Chemical, Combi-blocks, and others). To accomplish this despite the large quantity of data involved and its relative inaccessibility, we searched by substructure of all known heteroarenes. These final datasets were filtered to be low in price (<150$ per smallest bottle), not made on-demand, in-stock and not backordered, and being chemically compatible with Suzuki coupling (examples of compatible functional groups: hydroxyl, amine, ester, amide, etc.; examples of chemically incompatible functional groups: ketene, isocyanate, multiple reactive halogens). Through this process, the building blocks were narrowed from millions in databases and the literature, to hundreds of thousands listed by chemical suppliers, to tens of thousands of chemicals with prices, and finally >5000 currently purchasable and chemically compatible blocks. This final list is highly diverse and represents all chemical space currently accessible through heteroaryl Suzuki coupling. Importantly, the list contains examples of all heteroaryl substructures found in databases of drugs, natural products, and materials, with a variety of functional groups, and with or without protecting groups.

### Clustering and Selection of Representative Substrates.

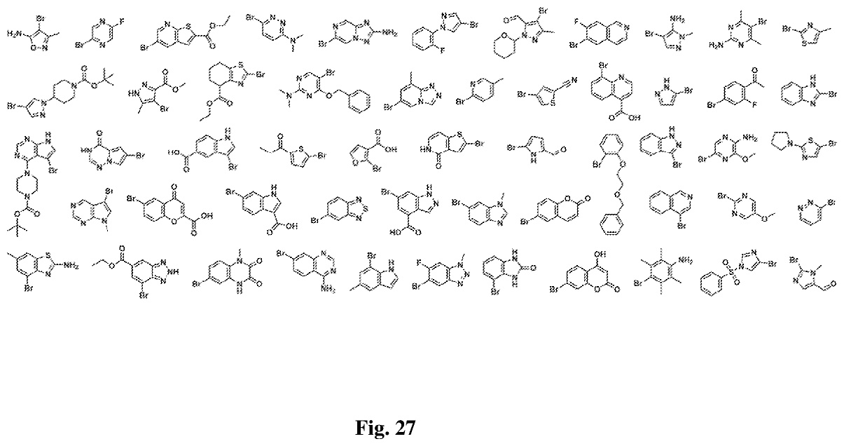

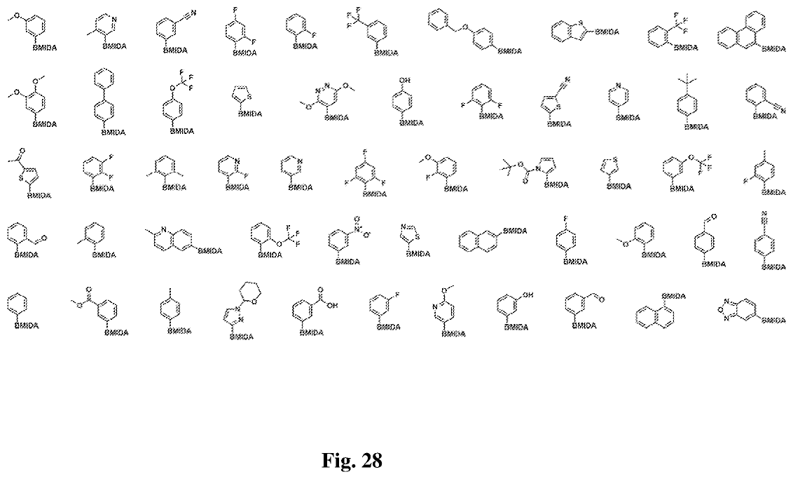

With the in silico building block library established, we needed to define a representative subset of this chemical space that could be practically purchased and stored in our laboratory for use in automated experiments. To do so, we applied a stratified clusterization strategy (Figure S21) to algorithmically cluster the commercial (hetero)aryl halides by their common (hetero) aromatic ring substructures and pendant functionalities, selecting molecules that most represented each section of the available chemical space. In doing so, we selected 54 (hetero)aryl halides which we then purchased (FIG. 27). We then selected an equal sized set of 54 (hetero)aryl MIDA boronates and purchased them (FIG. 28). As there are fewer commercially available (hetero)aryl MIDA boronates than halides, we did not apply a clusterization strategy for the selection of these building blocks but instead selected them on the basis of maximizing the representation of heteroaryl substructures. 2-pyridyl MIDA boronates were omitted from this selection process due to requiring additives not considered in subsequent optimization. Together, the building block combinations of the (hetero)aryl halide and MIDA boronates yield a substrate scope including >2900 unique products. Exploring all this space (multiplied by tens of possible conditions for each substrate pair; see below) is technically unfeasible—accordingly, we pursued the following strategy. Initially, we chose a small set of 11 substrate pairs (FIG. 3C) so as to minimize mutual similarity of the resulting products (which was achieved with a greedy algorithm based on the Tanimoto similarity). For these products we performed the (time consuming) standardization of UV-Vis spectra (LCMS-UV/Vis response factor curves, see Section 8 and 9) that then enabled us to determine the yields of the automatically performed reactions. These 11 pairs we tested under the initial set of conditions (FIG. 3E) and their subsets were tested under different conditions during the AI-guided-optimization phase. When this phase terminated, we then chose another set of 20 substrate pairs that were (i) maximally dissimilar to each other and (ii) maximally dissimilar to the 11 substrate pairs used during optimization (FIG. 5A). In other words, we tested the optimized general conditions on the test set of diverse molecules not seen during model training.

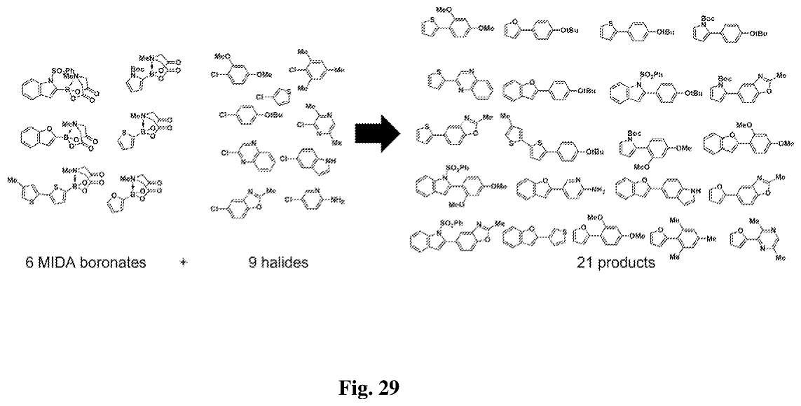

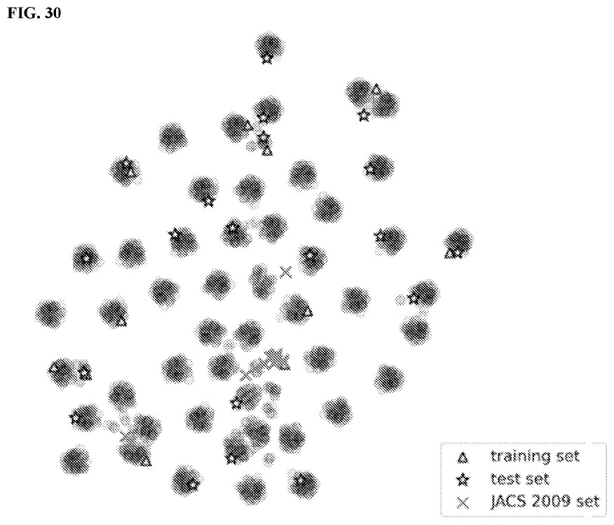

To compare this selection strategy against that employed during conventional reaction optimization, we examined the substrate scope (FIG. 29) of a widely utilized heteroaryl cross-coupling report used as a benchmark in this work. We plotted the products from JACS 2009 using T-distributed stochastic neighbor embedding (t-SNE) mapping along with the training and test sets examined in this work (FIG. 30). In this plot, it appears that the substrate scope from JACS 2009 primarily forms one cluster owing to the common occurrence of a smaller selection of chemical building blocks, whereas the training and test sets examined in this report remain spread out amongst the full space. This result suggests that reference JACS 2009 represents a comparatively small chemical space versus the training and test sets examined in this work, and that the latter more accurately represents the overall chemical space. This example highlights inherent drawbacks to the conventional reaction optimization process, mainly that the qualitative aspects of the substrate scope selection do not consider nor guarantee reaction generality. To further compare the two sets, a similarity metric was devised that is able to differentiate between pairs of building blocks differing in the placement of the halide vs. MIDA boronate (i.e., halide on substrate 1 and MIDA boronate on substrate 2 vs. halide on substrate 2 and MIDA boronate on substrate 1; this is highly chemically relevant due to the differing reactivity of these moieties depending on the molecular structure).

This metric worked as follows, using Tanimoto similarity with Morgan Fingerprints with radius=3 nBits=2048:
1. Similarity between 2 reactions was measured as: (similarity between 1^{st} bromide and 2^{nd} bromide)*(similarity between 1^{st} MIDA boronate and 2^{nd} MIDA boronate)
2. Calculated such similarity for each combination of pairs of substrates in the set
3. Calculated mean value from all calculated similarities

Following this approach yielded the following results:
Average similarity of the full product space examined in this work: 0.0314
Average similarity of the training set examined in this work: 0.0427
Average similarity of the substrate scope from reference JACS 2009:0.1136
According to this data, the training set examined in this work is similarly diverse to that of the full set (that it is supposed to represent) and is more diverse than the conventional substrate scope from reference JACS 2009.

Importantly, reference JACS 2009 is a useful benchmark for the state of the art in the field as it has been cited and its optimized protocol utilized widely. According to SCOPUS it has been cited by 440 scientific publications (98^{th} percentile) with a Field-Weighted citation impact of 7.67 (where 1 is average). Google Scholar, which includes patents, lists 594 citations. To illustrate its current relevance, we have included a graph from WebofScience (FIG. 31) which shows both the number of publications per year citing this work as well as the citations those papers receive (reproduced below). Generally, this shows the sustained interest and increasing relevance of this publication over time.

### Reaction Conditions Scope.

Regarding conditions, we considered four variables—solvent, base, catalyst/ligand and temperature—in whose selection we considered not only popularity in the literature but also diversity. For instance, while two most popular solvents in the literature are dioxane and dimethoxyethane, they both belong to the same solvent class of ethers, and we selected only one of them, dioxane. Similar reasoning led us to keep only one carbonate base (previously, we showed that nature of the cation did not alter the yields) whereas functional similarity of certain catalysts described in detail in the next section allowed us to eliminate some catalysts. Regarding temperatures, we selected 100° C. as the most popular in the literature as well as 60° C. which was used in the optimal conditions JACS 2009 paper against which we are benchmarking our current results. In the end, we selected three solvents (dioxane, toluene, dimethylformamide, each used in 5:1 mixture with water), two bases (sodium carbonate, potassium phosphate), two temperatures (60° C. and 100° C.) and seven catalysts (Pd SPhos G4, Pd(PPh₃)₄, Pd XPhos G4, Pd P(tBu)₃ G4, Pd PCy3 G4, Pd₂(dba)₃, and Pd(dppf)Cl₂). Consequently, we considered the space of 3×2×2×7=84 conditions.

Other reaction variables not explicitly varied in this optimization are: molar ratio of MIDA boronate to halide, catalyst loading, molar ratio of base, concentration, ratio of organic solvent to water, reaction time, stir rate, and metal to ligand ratio of the catalyst. These variables were not explored primarily due to practical considerations: each additional variable increases the total search space by approximately an order of magnitude as the reaction conditions space is multiplied by the full substrate space (11 substrate pairs). In general, the variables selected to test (solvent, base, catalyst/ligand, and temperature) are often important in influencing the outcome of the reactions, most commonly varied by bench chemists when optimizing individual reactions, and are often important in a discovery chemistry context. Each of these required components affects the elementary bond-forming steps of the reaction in some way:
1. The solvent solubilizes components, fills open coordination sites on the catalyst, influences speciation, stabilizes charges, and so on
2. The base influences boronic acid speciation and is necessary for subsequent transmetalation to the palladium oxidative addition complex
3. The ligands affect the steric and electronic environment around the palladium complex and affect the relative kinetics of the different steps in the catalytic cycle
4. The temperature provides energy to overcome the energetic barriers of the process, including influencing the relative rates of productive and unproductive pathways.
   Variables such as reaction time, concentration, and molar equivalence of reagents and catalyst are important in the context of scale up/process optimization, to limit waste. In the discovery context, it is more beneficial to use a longer reaction time and slight excess of the less stable reacting partner to ensure complete conversion of the starting materials, as the relative reaction rates for newly synthesized compounds are not known a priori and doing so generally maximizes the yield of the desired product.

### Example 6—Automated Synthesis

### Training Set—General Procedure 1 (GP1)

Following the general methods for automated synthesis, a 40 mL I-Chem vial equipped with a stir bar was charged with halide (0.1 mmol, 1 equiv), MIDA boronate (0.12 mmol, 1.2 equiv), Pd(PPh₃)₄ (5.8 mg, 0.005 mmol, 5 mol %), Na₂CO₃ (80 mg, 0.75 mmol, 7.5 equiv) and phenanthrene (internal standard, 17.8 mg, 0.1 mmol, 1 equiv). The vial(s) was (were) loaded onto the synthesis machine and the automated synthesis procedure was executed. The synthesis machine then executed the automated Schlenk-line procedure with 10 cycles, solvent addition (8 mL of 5:1 dioxane: water), heating (100° C.) and stirring (300 rpm) for the 12 h of reaction time, and then stopped the reaction by lowering the hotplate temperature (20° C.) and ceasing stirring (0 rpm). Afterwards, the reactions were purified by preparative HPLC for molecular characterization and response factor curve generation.

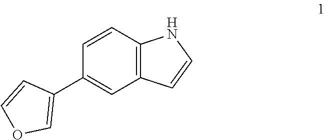 [SMILES: c1cc2cc(-c3ccoc3)ccc2[nH]1]

5-(Furan-3-yl)-1H-indole. Following GP1 using furan-3-MIDA boronate (27 mg, 0.12 mmol) and 5-bromo indole (20 mg, 0.1 mmol) gave 1 (80%) as a beige solid. 1H-NMR (500 MHz, acetone-d₆) δ 10.26 (br s, 1H), 7.93 (m, 1H), 7.81 (m, 1H), 7.61 (t, J=1.7 Hz, 1H), 7.45 (d, J=8.4 Hz, 1H), 7.38 (dd, J=8.4, 1.6 Hz, 1H), 7.35 (t, J=2.8 Hz, 1H), 6.90 (dd, J=1.8, 0.8 Hz, 1H), 6.49 (m, 1H); ¹³C-NMR (125 MHz, acetone-d₆) δ 143.5, 137.7, 135.7, 128.6, 127.8, 125.3, 123.6, 119.9, 117.3, 111.6, 109.0, 101.6; HRMS (ESI+) calculated for C₁₂H₁₀NO [M+H]⁺ m/z 184.0757, found 184.0754.

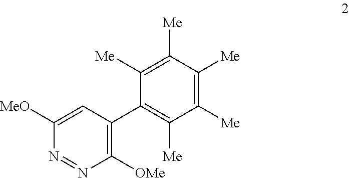 [SMILES: COc1cc(-c2c(C)c(C)c(C)c(C)c2C)c(OC)nn1]

3,6-Dimethoxy-4-(2,3,4,5,6-pentamethylphenyl)pyridazine. Following GP1 using 3,6-dimethoxypyridazine-4-MIDA boronate (35 mg, 0.12 mmol) and 1-bromo-2,3,4,5,6-pentamethylbenzene (23 mg, 0.1 mmol) gave 2 (84%) as a colorless crystalline solid. 1H-NMR (500 MHz, acetone-d₆) δ 6.77 (s, 1H), 4.05 (s, 3H), 3.93 (s, 3H), 2.26 (s, 3H), 2.22 (s, 6H), 1.89 (s, 6H); ¹³C-NMR (125 MHz, acetone-d₆) δ 162.3, 160.4, 135.8, 134.9, 132.2, 131.1, 130.6, 120.7, 53.8, 53.6, 17.1, 15.9, 15.6; HRMS (ESI+) calculated for C₁₇H₂₃N₂O₂ [M+H]⁺ m/z 287.1754, found 287.1746.

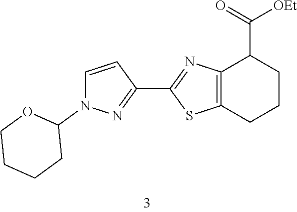 [SMILES: CCOC(=O)C1CCCc2sc(-c3ccn(C4CCCCO4)n3)nc21]

Ethyl-2-(1-(tetrahydro-2H-pyran-2-yl)-1H-pyrazol-3-yl)-4,5,6,7-tetrahydrobenzo[d]thiazole-4-carboxylate. Following GP1 using 1-(tetrahydro-2H-pyran-2-yl)-3-pyrazole MIDA boronate (37 mg, 0.12 mmol) and 2-bromo-4,5,6,7-tetrahydro benzothiazole-4-carboxylic acid ethyl ester (29 mg, 0.1 mmol) gave 3 (31%) as a colorless oil. 1H-NMR (500 MHz, acetone-d₆) δ 7.52 (d, J=1.8 Hz, 1H), 6.71 (d, J=1.8 Hz, 1H), 6.34 (dd, J=10.1, 2.3 Hz, 1H), 4.25-4.15 (m, 2H), 3.92-3.86 (m, 2H), 3.62 (td, J=11.3, 3.1 Hz, 1H), 2.97-2.86 (m, 2H), 2.49 (m, 1H), 2.19 (m, 1H), 2.15-2.08 (m, 3H), 1.99-1.86 (m, 2H), 1.75-1.53 (m, 3H), 1.28 (t, J=7.1 Hz, 3H); ¹³C-NMR (125 MHz, acetone-d₆) δ 172.8, 153.4, 148.2, 138.6, 135.9, 132.3, 108.0, 84.7, 67.1, 60.3, 43.4, 28.9, 26.7, 25.0, 22.9, 22.7, 21.0, 13.7; HRMS (ESI+) calculated for C₁₈H₂₄N₃O₃S [M+H]⁺ m/z 362.1533, found 362.1523.

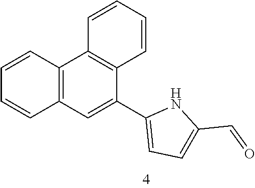 [SMILES: O=Cc1ccc(-c2cc3ccccc3c3ccccc23)[nH]1]

5-(Phenanthren-9-yl)-1H-pyrrole-2-carbaldehyde. Following GP1 using 9-phenanthrenyl MIDA boronate (40 mg, 0.12 mmol) and 5-bromo-1H-pyrrole-2-carbaldehyde (17 mg, 0.1 mmol) gave 4 (88%) as an off-white solid. 1H-NMR (500 MHz, acetone-d₆) δ 11.49 (br s, 1H), 9.68 (s, 1H), 8.94 (d, J=8.3 Hz, 1H), 8.87 (d, J=8.3 Hz, 1H), 8.26 (d, J=8.2 Hz, 1H), 8.04 (d, J=5.8 Hz, 2H), 7.79-7.74 (m, 2H), 7.72-7.67 (m, 2H), 7.24 (d, J=3.7 Hz, 1H), 6.70 (d, J=3.7 Hz, 1H); ¹³C-NMR (125 MHz, acetone-d₆) δ 178.5, 138.3, 133.9, 131.3, 130.7, 130.3, 130.3, 129.0, 128.6, 128.5, 127.5, 127.2, 127.0, 127.0, 126.2, 123.2, 122.7, 120.7, 112.4; HRMS (ESI+) calculated for C₁₉H₁₄NO [M+H]⁺ m/z 272.1070, found 272.1071.

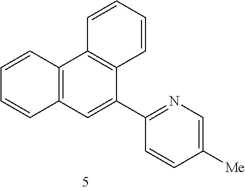 [SMILES: Cc1ccc(-c2cc3ccccc3c3ccccc23)nc1]

5-Methyl-2-(phenanthren-9-yl)pyridine. Following GP1 using 9-phenanthrenyl MIDA boronate (40 mg, 0.12 mmol) and 2-bromo-5-methylpyridine (17 mg, 0.1 mmol) gave 5 (43%) as a light-yellow oil. 1H-NMR (500 MHz, acetone-d₆) δ 8.93 (dd, J=8.3, 0.5 Hz, 1H), 8.88 (d, J=8.3 Hz, 1H), 8.65 (m, 1H), 8.19 (dd, J=8.3, 0.8 Hz, 1H), 8.05 (dd, J=7.8, 0.8 Hz, 1H), 7.91 (s, 1H), 7.81 (dd, J=7.9, 2.0 Hz, 1H), 7.77-7.71 (m, 2H), 7.69 (m, 1H), 7.65-7.59 (m, 2H), 2.48 (s, 3H); ¹³C-NMR (125 MHz, acetone-d₆) δ 156.4, 149.7, 137.5, 137.0, 131.8, 131.5, 130.7, 130.5, 130.3, 128.9, 128.0, 127.1, 127.0, 126.9, 126.6, 126.5, 124.3, 123.0, 122.6, 17.3; HRMS (ESI+) calculated for C₂₀H₁₆N [M+H]⁺ m/z 270.1277, found 270.1281.

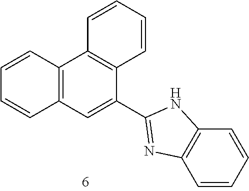 [SMILES: c1ccc2c(c1)cc(-c1nc3ccccc3[nH]1)c1ccccc12]

2-(Phenanthren-9-yl)-1H-benzo[d]imidazole. Following GP1 using 9-phenanthrenyl MIDA boronate (40 mg, 0.12 mmol) and 2-bromo-1H-benzimidazole (20 mg, 0.1 mmol) gave 6 (87%) as an off-white solid. 1H-NMR (500 MHz, DMSO d₆) δ 13.04 (s, 1H), 9.11 (dd, J=8.2, 1.2 Hz, 1H), 8.98 (d, J=8.0 Hz, 1H), 8.93 (d, J=8.3 Hz, 1H), 8.38 (s, 1H), 8.13 (d, J=7.0 Hz, 1H), 7.83-7.78 (m, 3H), 7.78-7.73 (m, 2H), 7.61 (d, J=7.4 Hz, 1H), 7.33-7.25 (m, 2H); ¹³C-NMR (125 MHz, DMSO d₆) δ 151.7, 144.3, 134.9, 130.9, 130.7, 130.7, 129.8, 129.6, 129.6, 128.6, 127.9, 127.7, 127.7, 127.6, 127.0, 123.7, 123.5, 123.2, 122.1, 119.6, 111.8; HRMS (ESI+) calculated for C₂₁H₁₅N₂ [M+H]⁺ m/z 295.1230, found 295.1229.

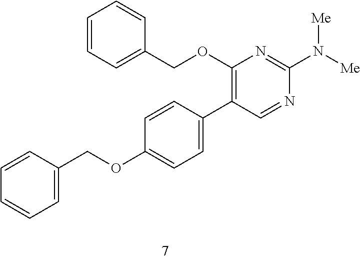 [SMILES: CN(C)c1ncc(-c2ccc(OCc3ccccc3)cc2)c(OCc2ccccc2)n1]

4-(Benzyloxy)-5-(4-(benzyloxy)phenyl)-N,N-dimethylpyrimidin-2-amine. Following GP1 using 4-(benzyloxy)phenyl MIDA boronate (41 mg, 0.12 mmol) and 4-benzyloxy-5-bromo-2-(N,N-dimethylamino)pyrimidine (31 mg, 0.1 mmol) gave 7 (88%) as a beige solid. 1H-NMR (500 MHz, acetone-d₆) δ 8.17 (s, 1H), 7.52-7.47 (m, 6H), 7.43-7.30 (m, 6H), 7.05 (dd, J=8.7, 1.8 Hz, 2H), 5.51 (s, 2H), 5.15 (s, 2H), 3.19 (s, 6H); ¹³C-NMR (125 MHz, acetone-d₆) δ 165.6, 161.2, 157.8, 157.4, 137.6, 137.6, 129.5, 128.4, 128.3, 127.7, 127.7, 127.6, 127.5, 127.3, 114.6, 109.3, 69.5, 67.0, 36.2; HRMS (ESI+) calculated for C₂₆H₂₆N₃O₂ [M+H]⁺ m/z 412.2020, found 412.2015.

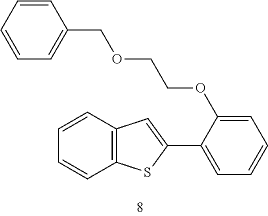 [SMILES: c1ccc(COCCOc2ccccc2-c2cc3ccccc3s2)cc1]

2-(2-(2-(Benzyloxy) ethoxy)phenyl)benzo[b]thiophene. Following GP1 using benzothiophene-2-MIDA boronate (35 mg, 0.12 mmol) and 1-(2-(benzyloxy) ethoxy)-2-bromobenzene (31 mg, 0.1 mmol) gave 8 (65%) as a light brown oil. 1H-NMR (500 MHz, acetone-d₆) δ 8.03 (s, 1H), 7.90 (m, 1H), 7.79 (dd, J=7.7, 1.6 Hz, 1H), 7.74 (m, 1H), 7.41 (d, J=7.1 Hz, 2H), 7.38-7.26 (m, 6H), 7.21 (d, J=8.3 Hz, 1H), 7.08 (td, J=7.5, 1.3 Hz, 1H), 4.70 (s, 2H), 4.40 (t, J=4.7 Hz, 2H), 4.01 (t, J=4.7 Hz, 2H); ¹³C-NMR (125 MHz, acetone-d₆) δ 155.8, 140.4, 140.0, 139.6, 138.8, 129.4, 129.1, 128.2, 127.5, 127.3, 124.2, 124.2, 123.5, 123.0, 122.8, 121.7, 121.1, 113.1, 72.8, 68.7, 68.2; HRMS (ESI+) calculated for C₂₃H₂₁O₂S [M+H]⁺ m/z 361.1257, found 361.1250.

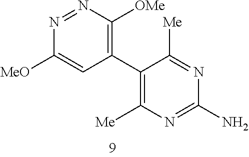 [SMILES: COc1cc(-c2c(C)nc(N)nc2C)c(OC)nn1]

5-(3,6-Dimethoxypyridazin-4-yl)-4,6-dimethylpyrimidin-2-amine. Following GP1 using 3,6-dimethoxypyridazine-4-MIDA boronate (35 mg, 0.12 mmol) and 5-bromo-4,6-dimethylpyrimidin-2-amine (20 mg, 0.1 mmol) gave 9 (24%) as an off-white solid, 1H-NMR (500 MHz, DMSO d₆) § 7.20 (s, 1H), 6.65 (s, 2H), 3.98 (s, 3H), 3.93 (s, 3H), 1.96 (s, 6H); ¹³C-NMR (125 MHz, DMSO d₆) δ 164.8, 163.0, 162.3, 160.2, 131.5, 122.2, 114.4, 54.9, 54.6, 22.6; HRMS (ESI+) calculated for C₁₂H₁₆N₅O₂ [M+H]⁺ m/z 262.1299, found 262.1295.

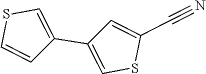 [SMILES: N#Cc1cc(-c2ccsc2)cs1]

[3,3′-Bithiophene]-5-carbonitrile. Following GP1 using 2-thiophene MIDA boronate (29 mg, 0.12 mmol) and 4-bromothiophene-2-carbonitrile (19 mg, 0.1 mmol) gave 10 (71%) as an off-white solid. 1H-NMR (500 MHz, acetone-d₆) δ 8.24 (t, J=1.2 Hz, 1H), 8.13 (t, J=1.2 Hz, 1H), 7.86 (m, 1H), 7.60 (dd, J=5.0, 2.9 Hz, 1H), 7.57 (m, 1H); ¹³C-NMR (125 MHz, acetone-d₆) δ 138.0, 136.8, 135.2, 127.2, 127.0, 126.1, 121.8, 113.8, 109.9; HRMS (EI+) calculated for C₉H₅NS₂ [M]⁺ m/z 190.9863, found 190.9864.

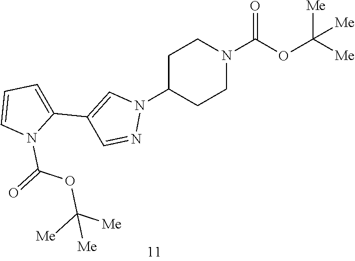 [SMILES: CC(C)(C)OC(=O)N1CCC(n2cc(-c3cccn3C(=O)OC(C)(C)C)cn2)CC1]

Tert-butyl 4-(4-(1-(tert-butoxycarbonyl)-1H-pyrrol-2-yl)-1H-pyrazol-1-yl) piperidine-1-carboxylate. Following GP1 using N-Boc-pyrrole-2-MIDA boronate (39 mg, 0.12 mmol) and 1-(4-Boc-piperidino)-4-bromopyrazole (33 mg, 0.1 mmol) gave 11 (47%) as a colorless oil. 1H-NMR (500 MHz, acetone-d₆) δ 7.82 (s, 1H), 7.55 (s, 1H), 7.30 (dd, J=3.3, 1.9 Hz, 1H), 6.20 (m, 2H), 4.40 (m, 1H), 4.21 (d, J=10.7 Hz, 2H), 2.96 (s, 2H), 2.09 (m, 2H), 1.94 (m, 2H), 1.51 (s, 9H), 1.48 (s, 9H); ¹³C-NMR (125 MHz, acetone-d₆) δ 154.1, 149.1, 138.9, 127.1, 126.9, 121.9, 113.8, 113.3, 110.4, 83.2, 78.7, 58.7, 42.6, 32.3, 27.7, 27.1; HRMS (ESI+) calculated for C₂₂H₃₃N₄O₄ [M+H]⁺ m/z 417.2496, found 417.2483.

### Test Set—General Procedure 2 (GP2)

Following the general methods for automated synthesis (Section 1), a 40 mL I-Chem vial equipped with a stir bar was charged with halide (0.1 mmol, 1 equiv), MIDA boronate (0.12 mmol, 1.2 equiv), Pd XPhos G4 (4.3 mg, 0.005 mmol, 5 mol %), Na₂CO₃ (80 mg, 0.75 mmol, 7.5 equiv) and phenanthrene (internal standard, 17.8 mg, 0.1 mmol, 1 equiv). The vial(s) was (were) loaded onto the synthesis machine and the automated synthesis procedure was executed. The synthesis machine then executed the automated Schlenk-line procedure with 10 cycles, solvent addition (8 mL of 5:1 dioxane: water), heating (100° C.) and stirring (300 rpm) for the 12 h of reaction time, and then stopped the reaction by cooling the hotplate (20° C.) and ceasing stirring (0 rpm). Afterwards, the reactions were purified by preparative HPLC for molecular characterization and response factor curve generation.

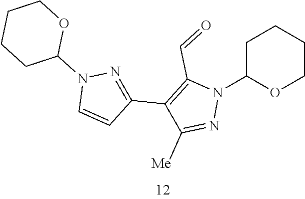 [SMILES: Cc1nn(C2CCCCO2)c(C=O)c1-c1ccn(C2CCCCO2)n1]

3′-Methyl-1,1′-bis(tetrahydro-2H-pyran-2-yl)-1H,1′H-[3,4′-bipyrazole]-5′-carbaldehyde. Following GP2 using 1-(tetrahydro-2H-pyran-2-yl)-3-pyrazole MIDA boronate (37 mg, 0.12 mmol) and 4-bromo-5-methyl-2-(oxan-2-yl) pyrazole-3-carbaldehyde (27 mg, 0.1 mmol) gave 12 as a colorless oil. 1H-NMR (500 MHz, acetone-d₆) δ 9.55 (s, 1br H), 7.62 (s, 1H), 6.43 (s, 1H), 6.17-6.11 (m, 1H), 5.07 (s, 1H), 4.00-3.97 (m, 1H), 3.84 (d, J=8.4 Hz, 1H), 3.75-3.70 (m, 1H), 3.45-3.31 (m, 1H), 2.49-2.36 (m, 2H), 2.14 (s, 3H), 2.12-2.08 (m, 1H), 2.04-1.96 (m, 2H), 1.89-1.83 (m, 1H), 1.80-1.72 (m, 1H), 1.65-1.47 (m, 5H); ¹³C-NMR (125 MHz, acetone-d₆) δ 180.1, 138.8, 137.3, 132.4, 109.2, 85.4, 85.3, 67.4, 67.4, 66.8, 24.9, 24.9, 24.8, 24.8, 22.5, 22.5, 22.3, 11.0; HRMS (ESI+) calculated for C₁₈H₂₄N₄NaO₃ [M+Na]⁺ m/z 367.1741, found 367.1737.

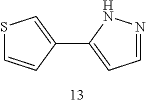

5-(Thiophen-3-yl)-1H-pyrazole. Following GP2 using 3-thiophene MIDA boronate (29 mg, 0.12 mmol) and 5-bromo-1H-pyrazole (15 mg, 0.1 mmol) gave 13 as an off-white solid. 1H-NMR (500 MHz, acetone-d₆) δ 12.16 (s, 1H), 7.73 (dd, J=2.8, 1.0 Hz, 1H), 7.68 (d, J=1.7 Hz, 1H), 7.56 (dd, J=5.0, 1.1 Hz, 1H), 7.51 (dd, J=5.0, 2.9 Hz, 1H), 6.61 (d, J=2.2 Hz, 1H); ¹³C-NMR (125 MHz, CDCl₃) δ 145.3, 133.5, 132.8, 126.3, 125.9, 121.1, 103.0; HRMS (ESI+) calculated for C—H₇N2S [M+H]⁺ m/z 151.0324, found 151.0326.

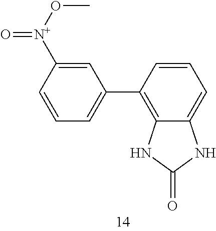 [SMILES: CO[N+](=O)c1cccc(-c2cccc3[nH]c(=O)[nH]c23)c1]

4-(3-Nitrophenyl)-1,3-dihydro-2H-benzo[d]imidazol-2-one. Following GP2 using 3-nitrophenyl MIDA boronate (33 mg, 0.12 mmol) and 4-bromo-1,3-dihydro-2H-benzimidazol-2-one (21 mg, 0.1 mmol) gave 14 as a yellow solid. 1H-NMR (500 MHz, DMSO d₆) δ 10.90 (s, 2H), 8.29 (s, 1H), 8.21 (d, J=8.0 Hz, 1H), 7.98 (d, J=7.5 Hz, 1H), 7.75 (t, J=7.9 Hz, 1H), 7.08 (d, J=4.1 Hz, 2H), 7.01 (m, 1H); ¹³C-NMR (125 MHz, DMSO d₆) δ 156.1, 148.6, 139.2, 135.2, 130.9, 130.8, 127.7, 123.5, 122.5, 121.7, 121.1, 120.8, 109.1; HRMS (ESI+) calculated for C₁₃H₁₀N₃O₃ [M+H]⁺ m/z 256.0717, found 256.0711.

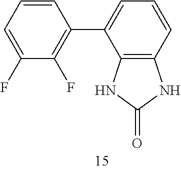 [SMILES: O=c1[nH]c2cccc(-c3cccc(F)c3F)c2[nH]1]

4-(2,3-Difluorophenyl)-1,3-dihydro-2H-benzo[d]imidazol-2-one. Following GP2 using (2,3-difluorophenyl) MIDA boronate (32 mg, 0.12 mmol) and 4-bromo-1,3-dihydro-2h-benzimidazol-2-one (21 mg, 0.1 mmol) gave 15 as an off-white solid. 1H-NMR (500 MHz, acetone-d₆) δ 10.08 (s, 2H), 7.39-7.33 (m, 1H), 7.33-7.28 (m, 2H), 7.16-7.11 (m, 2H), 7.07-7.03 (m, 1H). ¹³C-NMR (125 MHz, acetone-d₆) δ 155.5, 151.0 (dd, J=246.2, 13.0 Hz), 148.0 (dd, J=247.5, 13.1 Hz), 130.1, 128.3, 127.6 (d, J=12.3 Hz), 126.4-126.1 (m), 124.8 (dd, J=7.4, 4.7 Hz), 122.0 (d, J=0.7 Hz), 121.1, 116.5 (d, J=17.4 Hz), 116.2 (d, J=2.7 Hz), 108.9; 19F-NMR (471 MHz, acetone-d₆) δ-139.7 (m, 1F), −140.9 (m, 1F); HRMS (ESI+) calculated for C₁₃H₉F₂N₂O [M+H]⁺ m/z 247.0677, found 247.0678.

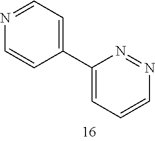 [SMILES: c1cnnc(-c2ccncc2)c1]

3-(Pyridin-4-yl)pyridazine. Following GP2 using 4-pyridine MIDA boronate (28 mg, 0.12 mmol) and 3-bromopyridazine (16 mg, 0.1 mmol) gave 16 as a white solid. 1H-NMR (500 MHz, acetone-d₆) δ 9.31 (dd, J=4.9, 1.5 Hz, 1H), 8.79 (dd, J=4.5, 1.7 Hz, 2H), 8.31 (dd, J=8.6, 1.5 Hz, 1H), 8.15 (dd, J=4.5, 1.7 Hz, 2H), 7.86 (dd, J=8.6, 5.0 Hz, 1H); ¹³C-NMR (125 MHz, acetone-d₆) δ 157.0, 151.4, 150.6, 143.8, 127.4, 124.3, 120.9; HRMS (ESI+) calculated for C₉H₈N₃ [M+H]⁺ m/z 158.0713, found 158.0717.

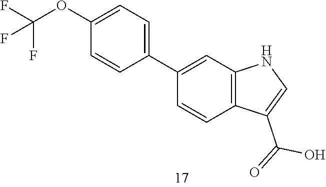 [SMILES: O=C(O)c1c[nH]c2cc(-c3ccc(OC(F)(F)F)cc3)ccc12]

6-(4-(Trifluoromethoxy)phenyl)-1H-indole-3-carboxylic acid. Following GP2 using 4-(trifluoromethoxy)phenyl MIDA boronate (38 mg, 0.12 mmol) and 6-bromo-1H-indole-3-carboxylic acid (24 mg, 0.1 mmol) gave 17 as a beige solid, 1H-NMR (500 MHz, CD₃OD) δ 8.28 (d, J=8.4 Hz, 1H), 7.81 (s, 1H), 7.73 (d, J=8.6 Hz, 2H), 7.60 (d, J=1.1 Hz, 1H), 7.38 (dd, J=8.3, 1.6 Hz, 1H), 7.31 (d, J=8.4 Hz, 2H); ¹³C-NMR (125 MHz, CD₃OD) δ 173.3, 147.9 (q, J=1.9 Hz), 141.6, 137.3, 133.2, 130.4, 128.2, 126.8, 121.8, 120.9, 120.7 (q, J=255.2 Hz, CF₃), 119.2, 114.2, 109.3; ¹⁹F-NMR (471 MHz, CD₃OD) δ-59.5; HRMS (ESI+) calculated for C₁₆H₁₁F₃NO₃ [M+H]⁺ m/z 322.0686, found 322.0680.

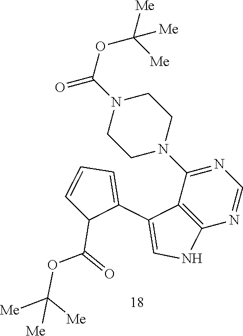 [SMILES: CC(C)(C)OC(=O)C1C=CC=C1c1c[nH]c2ncnc(N3CCN(C(=O)OC(C)(C)C)CC3)c12]

Tert-butyl-4-(5-(1-(tert-butoxycarbonyl)-1H-pyrrol-2-yl)-7H-pyrrolo[2,3-d]pyrimidin-4-yl) piperazine-1-carboxylate. Following GP2 using N-Boc-pyrrole-2-MIDA boronate (39 mg, 0.12 mmol) and 4-(4-Boc-1-piperazinyl)-5-bromo-7H-pyrrolo[2,3-d]pyrimidine (38 mg, 0.1 mmol) gave 18 as an off-white solid. 1H-NMR (500 MHz, acetone-d₆) δ 11.05 (s, 1H), 8.37 (s, 1H), 7.46 (dd, J=3.3, 1.9 Hz, 1H), 7.34 (s, 1H), 6.32 (t, J=3.3 Hz, 1H), 6.28 (dd, J=3.2, 1.9 Hz, 1H), 3.30 (s, 4H), 3.18 (s, 4H), 1.44 (s, 9H), 1.04 (s, 9H); ¹³C-NMR (125 MHz, acetone-d₆) δ 160.5, 154.0, 153.1, 150.6, 149.4, 128.6, 122.6, 121.8, 113.8, 110.4, 108.7, 106.5, 82.7, 78.9, 50.1, 49.2, 27.6, 26.3; HRMS (ESI+) calculated for C₂₄H₃₃N₆O₄ [M+H]⁺ m/z 469.2558, found 469.2557.

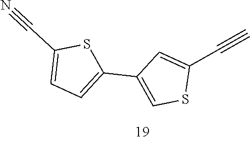

[2,3′-Bithiophene]-5,5′-dicarbonitrile. Following GP2 using 5-cyanothiophene-2-MIDA boronate (32 mg, 0.12 mmol) and 4-bromothiophene-2-carbonitrile (19 mg, 0.1 mmol) gave 19 as an off-white solid. 1H-NMR (500 MHz, acetone-d₆) δ 8.35 (d, J=1.5 Hz, 1H), 8.29 (d, J=1.5 Hz, 1H), 7.88 (d, J=4.0 Hz, 1H), 7.65 (d, J=4.0 Hz, 1H); ¹³C-NMR (125 MHz, acetone-d₆) δ 143.6, 139.2, 136.3, 134.0, 129.8, 125.7, 113.5, 113.2, 111.3, 108.2; HRMS (EI+) calculated for C₁₀H₄N₂S₂ [M]⁺ m/z 215.9816, found 215.9821.

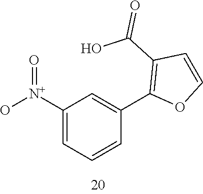

2-(3-Nitrophenyl) furan-3-carboxylic acid. Following GP2 using 3-nitrophenyl MIDA boronate (33 mg, 0.12 mmol) and 2-bromofuran-3-carboxylic acid (19 mg, 0.1 mmol) gave 20 as a yellow solid. 1H-NMR (500 MHz, CD₃OD) δ 8.96 (t, J=1.9 Hz, 1H), 8.45 (m, 1H), 8.15 (m, 1H), 7.62 (t, J=8.1 Hz, 1H), 7.54 (d, J=1.8 Hz, 1H), 6.78 (d, J=1.8 Hz, 1H); ¹³C-NMR (125 MHz, CD₃OD) δ 170.6, 149.2, 148.3, 141.4, 132.6, 132.4, 128.9, 123.4, 121.6, 121.2, 113.8; HRMS (ESI+) calculated for C₁₁H₆NO₅ [M−H]⁺ m/z 232.0251, found 232.0241.

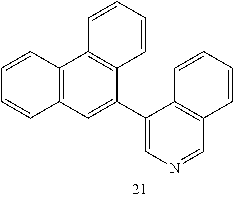 [SMILES: c1ccc2c(-c3cc4ccccc4c4ccccc34)cncc2c1]

4-(Phenanthren-9-yl) isoquinoline. Following GP2 using 9-phenanthrenyl MIDA boronate (40 mg, 0.12 mmol) and 4-bromoisoquinoline (21 mg, 0.1 mmol) gave 21 as an off-white solid. 1H-NMR (500 MHz, CDCl₃) δ 9.43 (s, 1H), 8.84 (dd, J=13.9, 8.3 Hz, 2H), 8.67 (s, 1H), 8.14 (d, J=8.2 Hz, 1H), 7.95 (dd, J=7.8, 0.7 Hz, 1H), 7.83 (s, 1H), 7.77 (m, 1H), 7.72-7.64 (m, 3H), 7.56 (m, 1H), 7.49 (d, J=8.4 Hz, 1H), 7.46-7.43 (m, 2H); ¹³C-NMR (125 MHz, CDCl₃) δ 152.5, 143.7, 135.7, 133.2, 131.9, 131.8, 131.4, 130.7, 130.5, 130.4, 129.2, 128.8, 128.2, 127.9, 127.4, 127.2, 127.1, 127.1, 126.8, 125.5, 123.0, 122.7; HRMS (ESI+) calculated for C₂₃H₁₆N [M+H]⁺ m/z 306.1277, found 306.1272.

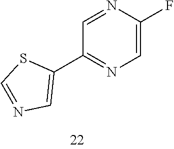 [SMILES: Fc1cnc(-c2cncs2)cn1]

5-(5-Fluoropyrazin-2-yl) thiazole. Following GP2 using 5-thiazole MIDA boronate (29 mg, 0.12 mmol) and 2-bromo-5-fluoropyrazine (18 mg, 0.1 mmol) gave 22 as an off-white solid. 1H-NMR (500 MHz, acetone-d₆) δ 9.15 (s, 1H), 8.93 (t, J=1.4 Hz, 1H), 8.64 (s, 1H), 8.60 (dd, J=8.3, 1.3 Hz, 1H); ¹³C-NMR (125 MHz, acetone-d₆) δ 159.5 (d, J=250.6 Hz), 155.8, 144.7 (d, J=4.6 Hz), 141.6 (d, J=1.4 Hz), 138.1 (d, J=10.1 Hz), 135.9 (d, J=1.8 Hz), 132.9 (d, J=38.9 Hz); ¹⁹F-NMR (471 MHz, acetone-d₆) δ-84.57 (d, J=7.8 Hz); HRMS (ESI+) calculated for C₇H₅FN₃S [M+H]⁺ m/z 182.0183, found 182.0184.

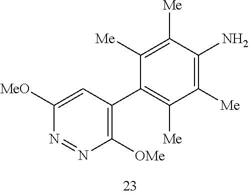 [SMILES: COc1cc(-c2c(C)c(C)c(N)c(C)c2C)c(OC)nn1]

4-(3,6-Dimethoxypyridazin-4-yl)-2,3,5,6-tetramethylaniline. Following GP2 using 3,6-dimethoxypyridazine-4-MIDA boronate (35 mg, 0.12 mmol) and 4-bromo-2,3,5,6-tetramethylaniline (23 mg, 0.1 mmol) gave 23 as a white solid, 1H-NMR (500 MHz, acetone-d₆) δ 6.74 (s, 1H), 4.30 (s, 2H), 4.03 (s, 3H), 3.91 (s, 3H), 2.11 (s, 6H), 1.87 (s, 6H); ¹³C-NMR (125 MHz, acetone-d₆) δ 162.3, 161.0, 144.0, 136.4, 130.7, 122.9, 121.2, 117.3, 53.7, 53.6, 16.9, 12.7; HRMS (ESI+) calculated for C₁₆H₂₂N₃O₂ [M+H]⁺ m/z 288.1707, found 288.1702.

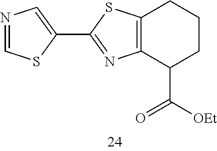 [SMILES: CCOC(=O)C1CCCc2sc(-c3cncs3)nc21]

Ethyl 2-(thiazol-5-yl)-4,5,6,7-tetrahydrobenzo[d]thiazole-4-carboxylate. Following GP2 using 5-thiazole MIDA boronate (29 mg, 0.12 mmol) and 2-bromo-4,5,6,7-tetrahydro-benzothiazole-4-carboxylic acid ethyl ester (29 mg, 0.1 mmol) gave 24 as an off-white solid. 1H-NMR (500 MHz, CDCl₃) δ 8.81 (s, 1H), 8.22 (s, 1H), 4.29-4.19 (m, 2H), 3.92 (t, J=5.8 Hz, 1H), 2.92 (m, 1H), 2.83 (m, 1H), 2.22 (m, 1H), 2.15-2.02 (m, 2H), 1.91 (m, 1H), 1.32 (t, J=7.1 Hz, 3H); ¹³C-NMR (125 MHz, CDCl₃) δ 173.3, 155.1, 153.7, 148.0, 141.5, 133.5, 132.2, 61.0, 43.1, 26.7, 23.4, 20.9, 14.3; HRMS (ESI+) calculated for C₁₃H₁₅N₂O₂S₂ [M+H]⁺ m/z 295.0569, found 295.0563.

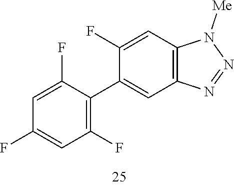 [SMILES: Cn1nnc2cc(-c3c(F)cc(F)cc3F)c(F)cc21]

6-Fluoro-1-methyl-5-(2,4,6-trifluorophenyl)-1H-benzo[d][1,2,3]triazole. Following GP2 using 2,4,6-trifluorophenyl MIDA boronate (34 mg, 0.12 mmol) and 5-bromo-6-fluoro-1-methyl-1,2,3-benzotriazole (23 mg, 0.1 mmol) gave 25 as a beige solid. 1H-NMR (600 MHz, acetone-d₆) δ 8.03 (dd, J=6.1, 0.3 Hz, 1H), 7.64 (dd, J=9.0, 0.3 Hz, 1H), 7.05-6.99 (m, 2H), 4.26 (s, 3H); ¹³C {¹H}-NMR (150 MHz, acetone-d₆) δ 163.8 (t, J=15.7 Hz), 162.1 (t, J=15.7 Hz), 161.5 (dd, J=15.7, 9.5 Hz), 159.9 (dd, J=15.7, 9.4 Hz), 158.3, 142.4, 134.3 (d, J=14.2 Hz), 123.1 (d, J=4.5 Hz), 113.7 (d, J=22.0 Hz), 109.0 (td, J=21.0, 4.8 Hz), 100.7-100.3 (m), 96.4 (d, J=29.3 Hz), 33.9; ¹³C {¹⁹F}-NMR (150 MHz, acetone-d₆) δ 162.9 (t, J_{C—H}=6.3 Hz), 160.7 (t, J_{C—H}=3.5 Hz), 159.1 (dd, J_{C—H}=10.1, 5.9 Hz), 142.4 (d, J_{C—H}=5.2 Hz), 134.3 (dt, J_{C—H}=7.7, 2.1 Hz), 123.7 (d, J_{C—H}=0.9 Hz), 122.6 (d, J_{C—H}=0.9 Hz), 113.7 (d, J_{C—H}=4.8 Hz), 109.0 (q, J_{C—H}=4.9 Hz), 101.1 (d, J_{C—H}=4.2 Hz), 100.0 (d, J_{C—H}=4.2 Hz), 97.0 (d, J_{C—H}=1.3 Hz), 95.9 (d, J_{C—H}=1.3 Hz), 33.9 (q, J_{C—H}=141.8 Hz); ¹⁹F-NMR (471 MHz, acetone-d₆) δ-108.6 (m, 1F), −110.9 (m, 2F), −115.3 (m, 1F); HRMS (ESI+) calculated for C₁₃H₈F₄N₃ [M+H]⁺ m/z 282.0649, found 282.0643.

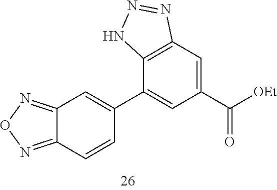 [SMILES: CCOC(=O)c1cc(-c2ccc3nonc3c2)c2[nH]nnc2c1]

Ethyl-7-(benzo[c][1,2,5]oxadiazol-5-yl)-1H-benzo[d][1,2,3]triazole-5-carboxylate. Following GP2 using 5-benzofurazan MIDA boronate (33 mg, 0.12 mmol) and ethyl 7-bromo-1H-1,2,3-benzotriazole-5-carboxylate (27 mg, 0.1 mmol) gave 26 as a light-yellow solid. 1H-NMR (500 MHz, acetone-d₆) δ 8.83 (s, 1H), 8.68 (s, 1H), 8.39 (d, J=5.5 Hz, 1H), 8.35 (d, J=9.3 Hz, 1H), 8.15 (m, 1H), 4.47 (qd, J=7.1, 1.8 Hz, 2H), 1.44 (t, J=7.1 Hz, 3H); ¹³C-NMR (125 MHz, acetone-d₆) δ 165.4, 149.7, 148.8, 142.0, 140.1, 138.6, 133.6, 128.5, 127.4, 124.1, 117.2, 116.5, 115.6, 61.2, 13.7; HRMS (ESI+) calculated for C₁₅H₁₂N₅O₃ [M+H]⁺ m/z 310.0935, found 310.0927.

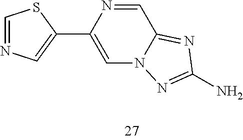 [SMILES: Nc1nc2cnc(-c3cncs3)cn2n1]

6-(Thiazol-5-yl)-[1,2,4]triazolo[1,5-a]pyrazin-2-amine. Following GP2 using 5-thiazole MIDA boronate (29 mg, 0.12 mmol) and 6-bromo-[1,2,4]triazolo[1,5-a]pyrazin-2-amine (21 mg, 0.1 mmol) gave 27 as a white solid. 1H-NMR (500 MHz, DMSO d₆) δ 9.46 (d, J=1.0 Hz, 1H), 9.12 (s, 1H), 8.85 (d, J=1.0 Hz, 1H), 8.56 (s, 1H), 6.61 (s, 2H); ¹³C-NMR (125 MHz, DMSO d₆) δ 167.7, 155.3, 146.1, 139.7, 137.5, 137.2, 133.2, 117.9; HRMS (ESI+) calculated for C₈H₇N₆S [M+H]⁺ m/z 219.0447, found 219.0452.

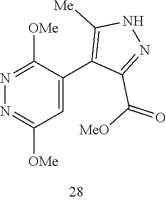 [SMILES: COC(=O)c1n[nH]c(C)c1-c1cc(OC)nnc1OC]

Methyl-4-(3,6-dimethoxypyridazin-4-yl)-5-methyl-1H-pyrazole-3-carboxylate. Following GP2 using 3,6-dimethoxypyridazine-4-MIDA boronate (35 mg, 0.12 mmol) and methyl 4-bromo-5-methyl-1H-pyrazole-3-carboxylate (22 mg, 0.1 mmol) gave 28 as a white solid. ¹H-NMR (500 MHz, CD₃OD) δ 7.05 (s, 1H), 4.04 (s, 3H), 3.95 (s, 3H), 3.77 (s, 3H), 2.25 (s, 3H); ¹³C-NMR (125 MHz, CD₃OD) δ 162.2, 160.1, 127.6, 121.1, 53.7, 53.7, 50.9, 8.5;* HRMS (ESI+) calculated for C₁₂H₁₅N₄O₄ [M+H]⁺ m/z 279.1088, found 279.1084.
* missing ¹³C resonances presumably due to tautomerization of the pyrazole.

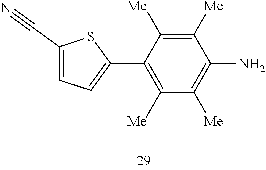 [SMILES: Cc1c(C)c(-c2ccc(C#N)s2)c(C)c(C)c1N]

5-(4-Amino-2,3,5,6-tetramethylphenyl)thiophene-2-carbonitrile. Following GP2 using 5-cyanothiophene-2-MIDA boronate (32 mg, 0.12 mmol) and 4-bromo-2,3,5,6-tetramethylaniline (23 mg, 0.1 mmol) gave 29 as an off-white solid. 1H-NMR (500 MHz, acetone-d₆) δ 7.85 (d, J=3.7 Hz, 1H), 6.90 (d, J=3.7 Hz, 1H), 4.46 (s, 2H), 2.12 (s, 6H), 2.00 (s, 6H); ¹³C-NMR (125 MHz, acetone-d₆) δ 153.6, 145.0, 138.3, 133.1, 128.3, 119.7, 117.3, 113.9, 108.6, 17.3, 12.8; HRMS (ESI+) calculated for C₁₅H₁₇N₂S [M+H]⁺ m/z 257.1107, found 257.1100.

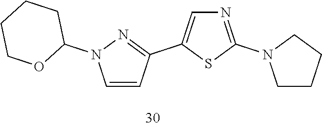 [SMILES: c1cn(C2CCCCO2)nc1-c1cnc(N2CCCC2)s1]

2-(Pyrrolidin-1-yl)-5-(1-(tetrahydro-2H-pyran-2-yl)-1H-pyrazol-3-yl) thiazole. Following GP2 using 1-(tetrahydro-2H-pyran-2-yl)-3-pyrazole MIDA boronate (37 mg, 0.12 mmol) and 5-bromo-2-pyrrolidinothiazole (23 mg, 0.1 mmol) gave 30 as a light-yellow oil. 1H-NMR (500 MHz, CDCl₃) δ 7.55 (d, J=1.8 Hz, 1H), 7.33 (s, 1H), 6.31 (d, J=1.8 Hz, 1H), 5.34 (dd, J=10.3, 2.4 Hz, 1H), 4.10 (m, 1H), 3.70 (td, J=11.6, 2.4 Hz, 1H), 3.54-3.50 (m, 4H), 2.58 (m, 1H), 2.11-2.08 (m, 4H), 1.95 (m, 1H), 1.76 (m, 2H), 1.68-1.57 (m, 2H); ¹³C-NMR (125 MHz, CDCl₃) δ 168.7, 140.0, 139.4, 135.2, 112.0, 107.1, 84.4, 67.6, 49.5, 29.3, 25.7, 24.9, 23.0; HRMS (ESI+) calculated for C₁₅H₂₁N₄OS [M+H]⁺ m/z 305.1431, found 305.1429.

### Closed-Loop Experiments—General Procedure 3 (GP3)

Following the general methods for automated synthesis (Section 1), a 40 mL I-Chem vial equipped with a stir bar was charged with halide (0.1 mmol, 1 equiv), MIDA boronate (0.12 mmol, 1.2 equiv), catalyst (0.005 mmol, 5 mol %), base (0.75 mmol, 7.5 equiv) and phenanthrene (internal standard, 17.8 mg, 0.1 mmol, 1 equiv). The selection of catalyst, base, solvent, and temperature per substrate was performed by the ML model. The vial(s) was (were) loaded onto the synthesis machine and the automated synthesis procedure was executed. The synthesis machine then executed the automated Schlenk-line procedure with 10 cycles, solvent addition (8 mL of solvent), heating, and stirring (300 rpm) for the 12 h of reaction time, and then stopped the reaction by lowering the hotplate temperature (20° C.) and ceasing stirring (0 rpm). Afterwards, the reactions were quantified by direct LCMS injection.

### Example 7—Closed-Loop Optimization and Post-hoc Analysis Initial Pool of Reactions

To “seed” the optimization procedure, the initial set of reactions was chosen without AI guidance as described above. We decided to perform couplings of all 11 substrate pairs defining our scope in 1,4-dioxane:water mixture as solvent (representing the most common class in the literature) and the following combinations of base and catalyst: i) sodium carbonate and Pd(PPh₃)₄ at 100° C. (the most common base, most common catalyst, and most common temperature in the literature) and ii) combinations of potassium phosphate (representative of the second most common base in the literature and used in the benchmark condition) with the following catalysts: Pd SPhos G4, Pd XPhos G4, Pd P(tBu)₃ G4, Pd PCy3 G4, Pd₂(dba)₃, and Pd(dppf)Cl₂, each with temperature set to 60° C. (used in the benchmark condition). With these choices, our robotic system was tasked with performing 11×7 reactions, each performed in duplicate. The average yields obtained in these experiments are tabulated in the main text FIG. 3E.

Of note, after this initial round, we observed that yields obtained for certain substrates with different pairs of ligands were quite similar—in other words, certain catalysts seemed to systematically give similar results and could thus be redundant. In order to quantify such functional similarity, we computed Spearman rank matrix (Main Text FIG. 3F) correlating yields obtained for all 11 substrate pairs using two different catalyst ligands—in this representation, redundant catalysts correspond to high-correlation, off-diagonal elements. In this particular case, XPhos catalysts was found “functionally similar” to Dppf, and PCy3 to SPhos—note that their “functional similarity” could not have been assessed simply based on the structural similarity which is largely lacking. Because of this result, we decided to eliminate PCy₃ and Dppf from our pool of ligands, in order to decrease redundancy. We also eliminated Pd₂(dba)₃ from our pool of catalysts due to poor performance (<5% yield for 8/11 substrates).

### Selection of Batch of Reactions in the Closed-Loop Experiments

Having chosen the model, selection strategies and initial dataset, we proceeded with real experiments. The space to explore comprised 11 substrate pairs multiplied by 48 conditions (2 bases×2 temperatures×3 solvents×4 catalysts). The navigation over this substrates-conditions space was guided by algorithms detailed in Section 3. One important aspect is that we worked in experimental batches meaning that multiple experiments were performed before the theoretical model was updated. Within each batch, the experiments were selected as the model's best-suggestion, second-best, third-best, etc., until the number of slots in the batch were taken. In more detail, the algorithm first formed a ‘priority que’ of unexplored reactions by i) sorting reaction conditions according to computed PI (in a descending order) and ii) sorting reactions (i.e., substrate pairs) under the same conditions according to the prediction uncertainty (again, in a descending order). Then, with and arbitrary number Nₛₑₗ of preferred conditions to select, we iteratively a) select the top-Nₛₑₗ conditions from the priority queue and b) append the top-most reaction from each of the selected conditions to the proposed batch, removing it from the queue. These selection iterations proceed until the batch reaches the desired batch size B. As our experimental setup allowed for performing 36 and later 72 reactions each week, we decided to take set Nₛₑₗ to 18. That is, each batch probed 18 different conditions and 2 or 4 pairs of substrates for each condition.

### Loop Termination

In order to determine when to terminate the loop, we monitored uncertainty of the model's predictions over both performed (‘seen’) and unexplored parts of the reaction-condition space. The general reasoning was that if the average uncertainty over the unexplored space drops to the level comparable with the set explored prior to a given generation (“training set”) (which in GP corresponds to the measurement uncertainty), then it is likely that the model gained sufficient knowledge about the whole space. Specifically, we monitored: a) average uncertainty over whole reaction space (both seen and unexplored), b) average uncertainty over the “training” set, and c) average uncertainty over unexplored part of reaction space (FIGS. 4A and 4F). As expected, we observed gradual reduction of model's uncertainty, indicating its increasing knowledge about the reaction-condition space. This progress slows down after the third iteration, when the average uncertainty on the unexplored part of the space approaches the value characterizing the training set.

At loop termination, the model had explored a total of 329 unique reactions: 77 duplicated from the seeding round 1, 36 duplicated from round 2, 72 from round 3, 72 from round 4, and 84 from round 5. Of these experiments, 33 from the seeding round used conditions outside of the set considered in the closed-loop optimization, as they contained catalysts which were functionally redundant (FIG. 3F). As the total space to explore was 48 conditions×11 substrates=528 reactions, the model explored 308/528 (58%) of the reactions. The models' selections of substrates to test per round is shown in FIG. S27.

Post-Hoc Analyses—Comparison with Random Selection Baseline

After uncertainty of our model plateaued around 3% (which coincides with the measurement uncertainty), we terminated our closed-loop experiments and post hoc compared the performance of our algorithm with a random-selection baseline by the simulation approach described in Section 3.3. To do so, we assumed that the model collected enough data to make trustworthy extrapolation to the unexplored part of the reaction-condition space and thus we took its prediction over the whole space as a ground truth. Since in the actual experiments, the batch size was adjusted after the first two iterations, we performed a simulation mimicking these circumstances, with results presented in FIG. 4B. These results indicate that identification of optimal conditions with random selection of the “next” experiments would require execution of nearly twice as many reactions as in the AI-guided scheme.

Post-Hoc Analyses—Comparison of Yield Distributions in Our Dataset Vs. The Set of Published Suzuki Couplings.

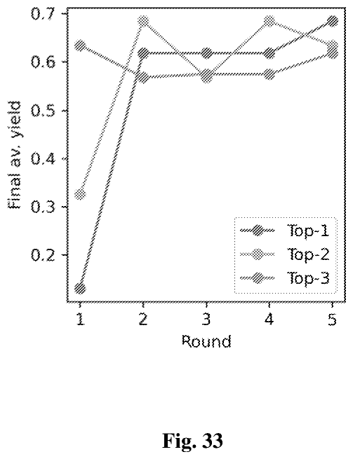

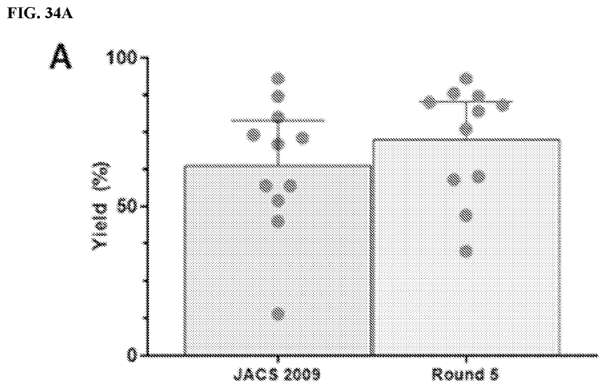

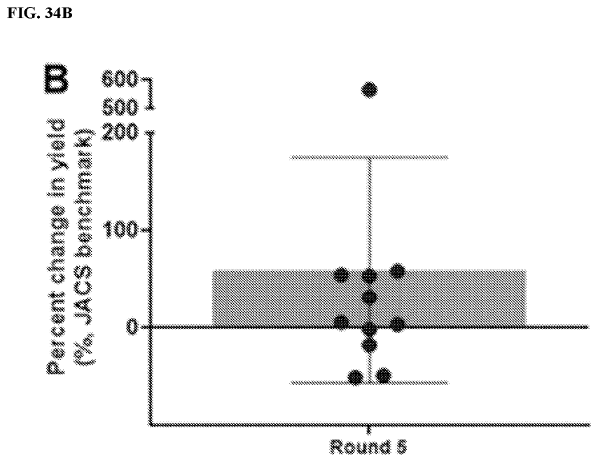

It is also instructive to compare the distribution of reaction yields recorded in our experiments against the distribution of yield in literature-reported Suzuki couplings performed under the same conditions (that is, combination of solvent, base and ligand also used in at least one of our experiments). As can be seen in Main Text FIG. 4C, reaction yields for our dataset are distributed more or less uniformly over the range of possible values, whereas in the case of literature-reported reactions it is strongly biased towards higher yield, with peak around 70-80% (i.e., close to the average yield of all literature-reported reactions). In other words, our protocol learned by probing both failed and high-yielding conditions whereas the published literature is dominated only by the positive outcomes (which, as we discussed in many of our previous works on computerized synthesis, limits the usefulness of approaches aiming to learn from literature data). Next, we examined the average yield of top-k predicted ‘general conditions’ in each iteration (FIG. 33 and Table 4). In the beginning, the model predicted ranking of the best ‘general’ conditions was incorrect but the relative performance of the top conditions gradually improved over the optimization and ultimately overtook the third-best condition that consistently gave good yields. The average yield for the JACS 2009 benchmark conditions was 64% and for the top ML-identified condition after round 5, the average yield was 72% (FIG. 34A and FIG. 34B). The superiority of the top ML conditions was ultimately confirmed by the additional 20 reactions on the out-of-box test set (FIG. 5).

TABLE 4
Top-1,2 and 3 conditions as predicted by the model
in subsequent rounds of closed-loop optimization.

| | | | | |
|---|---|---|---|---|
| round | solvent | base | ligand | temperature |

Top-1

| | | | | |
|---|---|---|---|---|
| 1 | Toluene:water | Na2CO3 | SPhos | 100 |
| 2 | 1,4-dioxane:water | Na2CO3 | PPh3 | 100 |
| 3 | 1,4-dioxane:water | Na2CO3 | PPh3 | 100 |
| 4 | 1,4-dioxane:water | Na2CO3 | PPh3 | 100 |
| 5 | 1,4-dioxane:water | Na2CO3 | XPhos | 100 |

Top-2

| | | | | |
|---|---|---|---|---|
| 1 | DMF:water | Na2CO3 | SPhos | 100 |
| 2 | 1,4-dioxane:water | Na2CO3 | XPhos | 100 |
| 3 | 1,4-dioxane:water | K3PO4 | SPhos | 60 |
| 4 | 1,4-dioxane:water | Na2CO3 | XPhos | 100 |
| 5 | 1,4-dioxane:water | Na2CO3 | SPhos | 100 |

Top-3

| | | | | |
|---|---|---|---|---|
| 1 | 1,4-dioxane:water | Na2CO3 | SPhos | 100 |
| 2 | 1,4-dioxane:water | K3PO4 | SPhos | 60 |
| 3 | 1,4-dioxane:water | K3PO4 | XPhos | 60 |
| 4 | 1,4-dioxane:water | K3PO4 | XPhos | 60 |
| 5 | 1,4-dioxane:water | Na2CO3 | PPh3 | 100 |

### Post-Hoc Analyses—Generalization of Conditions Optimized for Individual Reactions.

Here, we wish to evaluate a commonly used protocol entailing optimization of conditions for individual reaction(s) followed by generalization to a broader scope of substrates (we will refer to this “baseline” method as “independent-BO,” iBO, optimization strategy). In particular, we test a strategy in which one (1) selects N_{subs} substrate pairs from the pool of possible starting materials (we assume random selection, as there may not be pre-existing knowledge to drive the selection); (2) optimizes conditions (preferably with some Bayesian optimization approach) independently for each selected pair of starting materials; and (3) evaluates best-yielding conditions against other substrates in the pool. In testing this strategy, we used post hoc the data generated in our experiments as well 1536 data points. For step (2), we choose a recent Random Forest (RF) algorithm with 10 randomly selected conditions for RF initialization and a stop criterion chosen such as to terminate optimization if the best-solution found up to a given point is not improved within N_{wait} steps following its “discovery”. Regarding step (3), we aggregate all data collected during optimizations of N_{subs} individual reactions (i.e., all conditions-yield pairs) and estimate the average yield under given conditions. This is achieved by (i) grouping the collected data by reaction conditions (which results in assigning each reaction condition with a set of 1 to N_{subs} measured yields, depending on the number of optimization runs where they occurred), and (ii) averaging the yields within each group. Since some reaction conditions can be examined exclusively in optimizations of some subset of N_{subs} substrate pairs, there may be different numbers of measurements to score each reaction condition (e.g., just one yield measurement under conditions A vs. three measurements under conditions B). However, in the absence of a statistical model aiming to address this issue (which was the purpose of our work), we take these yield estimates as the best data available.

Note that steps 1-3 involve several sources of randomness: (i) the selection of N_{subs} pairs of starting materials (e.g., for the choice of 7 substrate pairs out of 11, there's 330 possible selections), (ii) selection of initial conditions to seed the BO optimizer for each reaction (as we have 48 conditions, there's about 6·10⁹ possible choices of the initial 10 reaction conditions used to seed the system) and (iii) the intrinsic randomness of the Random Forest algorithm (which may thus produce different optimization trajectories even with the same initial set of reactions). To average over these factors, we repeat steps (1)-(3)1000 times, which allows us to compute a probability that the “true” general conditions (i.e., those found by the algorithm described in the main text) are the same as the top conditions identified according to the empirical ranking derived from independent optimization of N_{subs} reactions (the top-1 statistic from steps (1)-(3)).

FIG. 35 summarizes the results of the iBO baseline on our calibration dataset (1536 Buchwald-Hartwig, BH, reactions, and on the heteroaryl Suzuki coupling (hetSMC) data collected in this study (with missing values filled with predictions of the final model), both in the function of two control variables: N_{subs} and N_{wait}. The heat maps in panels A and C quantify the top-1 statistic for BH and hetSMC datasets, respectively—in both cases, the iBO strategy is substantially worse than the optimization protocol described in the main paper. Indeed, for the BH set and irrespective of N_{wait}, top-1 values are merely ˜5-9% for N_{subs}=1 and ˜1-8% for N_{subs}=7. For the hetSMC dataset (with “narrower” conditions space), the performance is better: for N_{subs}=1 and N_{wait}=3, top-1 is only 15.7% and reaches 53.5% for N_{subs}=7 and N_{wait}=20 (which is in line with our intuition that using more substrates and reactions should improve the odds of finding the global optimum). Still, even with 297 reactions performed during the (N_{subs}=7; N_{wait}=20) optimization protocol, these odds are roughly a coin toss.

It is instructive to put these results in the context of the total number of reactions involved in each combination of N_{subs} and N_{wait} (FIG. 35B and FIG. 35D). Because in the iBO scheme we do not have control over total number of reactions (the optimization may end sooner or later, depending on which reactions are drawn for the initial pool), each tile actually corresponds to a distribution of the number of reactions, from which we select the maximum (upper bound) for visualization. As could be expected, this bound monotonically increases from bottom left to upper right for both BH and hetSMC datasets. For hetSMC, with N_{subs}=7 and N_{wai t}=20, the number of reactions to be performed (297) coincides with the total number of experiments executed in our study (˜300)—however, as we have discussed above, the iBO baseline has top-1 probability as good as a coin toss, whereas for our model, it converges to 1 (FIG. 4B).

Overall, these results indicate that independent optimization of individual reactions is significantly worse in finding the “generally optimal” conditions than the “coupled” method we described.

### Example 9—LCMS Quantification of Reaction Yield

### Percent Yield Calculation.

After completion of reaction, the reaction mixture was injected into an LCMS (undiluted, 1 μL). The relative ratio of the total wavelength count (TWC) UV/Vis integrated peak area between the product and internal standard (phenanthrene) was determined. This peak area ratio was multiplied by the slope obtained from the response factor curve of the corresponding compound to provide the percent yield.

### Example 10—Test Set Product and Byproduct Distribution

To understand the origin of the increased yield of the top discovered ML general reaction condition compared with the benchmark condition, we first probed extended reaction times for the benchmark condition to rule out incomplete reaction profiles due to the lower temperature of the benchmark condition (60° C.) vs the top ML condition (100° C.). To do so, we performed head-to-head experiments at 12, 24, and 36 hours of reaction time for five of the test set compounds which displayed the largest differences in yield between the benchmark and top ML condition (Figure S33). Interestingly, we observed that the yield of the reactions did not increase when employing longer reaction times. In fact, all the yields decreased slightly, presumably due to product degradation. This means that incomplete conversion at lower reaction temperatures is not the cause for the difference in reaction outcomes.

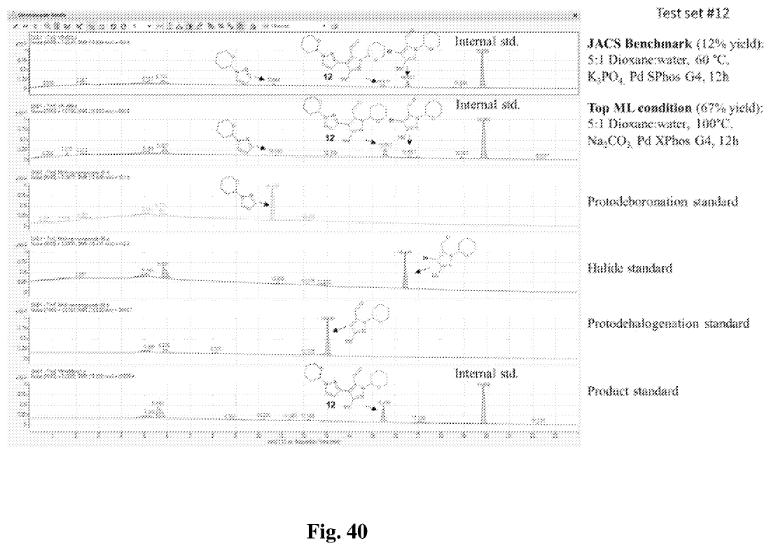

To understand why the yield increased under the top ML general condition, we quantified and compared the formation of all observable byproducts and remaining starting materials for the benchmark condition and the top ML condition for each of the substrates in the test set, and have included this information in FIG. 5E-G. Byproducts were identified by comparison to purchased authentic standards (if commercially available), ionization by ESI+LCMS (if observed with high mass accuracy and isotope scoring), or authentic standards synthesized independently (see synthesis details at the end of this section and structural characterization data in Section 7). Comparisons of product distributions of an exemplary test set substrate under the JACS benchmark and the top ML condition are presented in FIG. 40.

The length and broad polarity range of the HPLC gradient enabled the general separation and quantification of these compounds. There are some limitations to this data, notably the volatility of some byproducts (e.g., thiophene, thiazole) may cause evaporative loss to the argon manifold over the reaction time, especially at elevated temperatures. In addition, there are a few cases where retention times of byproducts cannot be distinguished from other reaction components, most notably in the case of product #19 where protodeboronation and protodehalogenation yield the same chemical structure. In addition, MIDA boronates, boronic acids, and homocoupling products are not observed in these reactions. The explanations for these observations are relatively straightforward: the MIDA boronates are all hydrolyzed during the reaction conditions, the boronic acids are all either consumed or protodeboronated by the reaction conditions, and homocoupling is precluded by the rigorous air-exclusion of the robotic experimental platform. With these aspects duly noted, this analysis demonstrated that the ML general conditions are higher yielding primarily due to a shift in product distribution away from byproducts and toward targeted product formation.

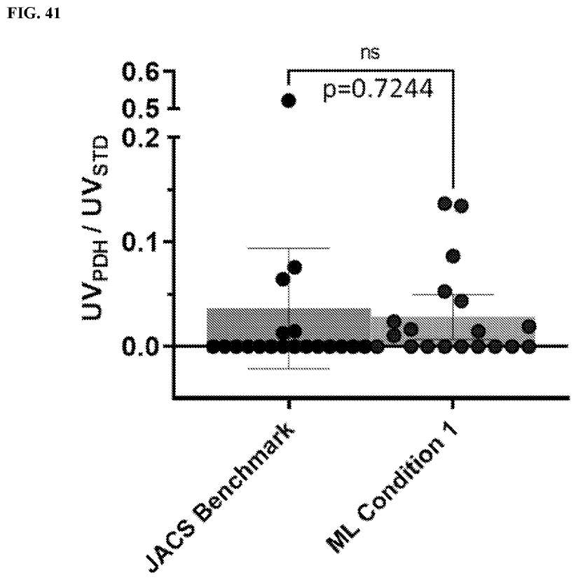

Differences are apparent between the general conditions for: protodeboronation (FIG. 5E), remaining halide starting material (FIG. 5F), and ratio of product formation vs all byproducts (FIG. 5G). For the latter, ‘all byproducts’ is defined as the sum of all peaks that can not be assigned to either the internal standard, the product, or the halide. Differences are not apparent between the general conditions regarding protodehalogenation, likely due to its relatively lower incidence (FIG. 41).

### Example 11—Byproduct Synthesis

The protodehalogenation standard for test set reaction 12 as well as 18 were not commercially available and were not detected by ESI+LCMS, and so these molecules were synthesized independently for the previous comparisons. Synthesis details are included below.

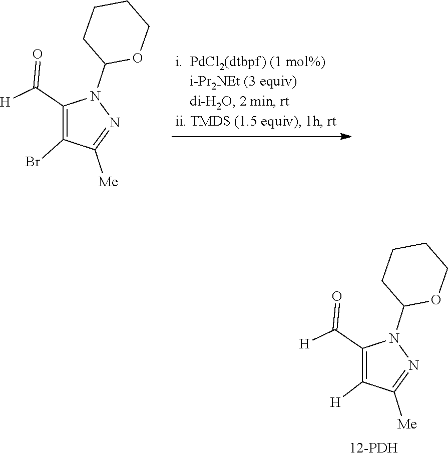 [SMILES: Cc1nn(C2CCCCO2)c(C=O)c1Br ; Cc1cc(C=O)n(C2CCCCO2)n1]

12—PDH was prepared following a modified literature procedure. To a 40 mL I-Chem vial with septa cap and equipped with a rare earth Teflon coated stir bar (10 mm diameter) was added 4-bromo-3-methyl-1-(tetrahydro-2H-pyran-2-yl)-1H-pyrazole-5-carbaldehyde (546 mg, 2 mmol), Pd (dtbpf) Cl₂ (13.4 mg, 0.02 mmol, 1 mol %), di-H₂O (8 mL), and N,N-diisopropylethylamine (1 mL, 6 mmol). The reaction was stirred for 2 min at rt followed by dropwise addition of 1,1,3,3-tetramethyldisiloxane (530 μL, 3 mmol). The vial was sealed and stirred at 500 rpm. After 1 h, the reaction was extracted with ethyl acetate (2×10 mL). The combined organic layers were dried over Na₂SO₄ and concentrated in vacuo (bath temp: 35° C.), in the presence of celite. The crude reaction mixture, adhered onto celite, was loaded onto a 10 g silica gel column with additional hexanes (1 mL) and purified by flash column chromatography (elution: 10→20% ethyl acetate/hexanes), yielding 12—PDH in the presence of siloxane impurities. 12—PDH was further purified by solid phase extraction: dry loading onto C18 silica gel (dissolution solvent: acetone) followed by elution off of the C18 silica gel (eluent: 1:1 water:acetonitrile) yielded pure 12—PDH as a colorless oil (47.4 mg, 0.244 mmol, 12% yield). 1H-NMR (500 MHz, CDCl₃) δ 9.85 (s, 1H), 6.70 (s, 1H), 6.07 (dd, J=10.3, 2.5 Hz, 1H), 4.12-4.04 (m, 1H), 3.72 (td, J=11.5, 2.5 Hz, 1H), 2.42-2.34 (m, 1H), 2.32 (s, 3H), 2.12-2.02 (m, 1H), 1.98-1.90 (m, 1H), 1.78-1.65 (m, 2H), 1.61-1.53 (m, 1H). ¹³C-NMR (125 MHz, CDCl₃) δ 179.9, 149.0, 139.8, 115.1, 85.6, 68.3, 29.9, 24.9, 22.7, 13.4; HRMS (EI+) calculated for C₁₀H₁₄N₂O₂ [M+]⁺ m/z 194.1055, found 194.1051.

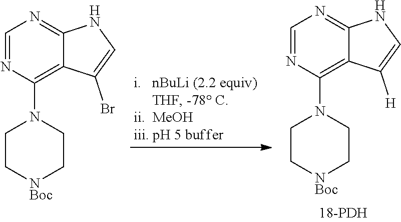 [SMILES: CC(C)(C)OC(=O)N1CCN(c2ncnc3[nH]cc(Br)c23)CC1 ; CC(C)(C)OC(=O)N1CCN(c2ncnc3[nH]ccc23)CC1]

To a flame-dried 40 mL I-Chem vial with septa cap and equipped with a rare earth Teflon coated stir bar (10 mm diameter) was added tert-butyl 4-(5-bromo-7H-pyrrolo[2,3-d]pyrimidin-4-yl) piperazine-1-carboxylate (197 mg, 0.515 mmol) and anhydrous THF (5 mL). The reaction was cooled to −78° C. and n-BuLi (1.6M in hexanes, 0.708 mL, 1.13 mmol) was added dropwise. The reaction was stirred for 5 min and methanol (50 μL, 1.24 mmol) was added. The reaction was stirred for 1 min, quenched by addition of 10 mL pH 5 phosphate buffer. The reaction was extracted with ethyl acetate (2×10 mL). The combined organic layers were dried over Na₂SO₄ and concentrated in vacuo (bath temp: 35° C.), in the presence of celite. The crude reaction mixture, adhered onto celite, was loaded onto a 10 g silica gel column and purified by flash column chromatography (elution: 50→75% ethyl acetate/hexanes [starting material elutes], then 75→90% ethyl acetate/hexanes [product elutes]), yielding 18—PDH as a white solid (42 mg, 0.138 mmol, 27% yield). ¹H-NMR (500 MHz, CDCl₃) δ 12.12 (s, 1H), 8.36 (s, 1H), 7.15 (d, J=3.7 Hz, 1H), 6.50 (d, J=3.7 Hz, 1H), 4.02-3.93 (m, 4H), 3.68-3.55 (m, 4H), 1.49 (s, 9H). ¹³C-NMR (125 MHz, CDCl₃) δ 157.1, 154.8, 152.2, 150.6, 121.1, 103.1, 101.0, 80.1, 45.3 (broad), 43.8 (broad), 42.8 (broad), 28.4; HRMS (ESI+) calculated for C₁₅H₂₂N₅O₂ [M+H]⁺ m/z 304.1773, found 304.1774.

### Example 12—Statistical Comparisons

### Dataset: FIG. 5B

| JACS | ML General | ML General | ML General |
|---|---|---|---|
| Benchmark | condition 1 | condition 2 | condition 3 |
| 59 | 65 | 60 | 65 |
| 6 | 41 | 22 | 43 |
| 0 | 35 | 0 | 3 |
| 80 | 59 | 63 | 7 |
| 68 | 62 | 33 | 38 |
| 11 | 16 | 24 | 7 |
| 18 | 44 | 46 | 57 |
| 16 | 60 | 30 | 60 |
| 19 | 16 | 20 | 7 |
| 53 | 81 | 60 | 45 |
| 10 | 17 | 14 | 3 |
| 5 | 33 | 15 | 17 |
| 6 | 86 | 86 | 82 |
| 7 | 76 | 61 | 33 |
| 12 | 67 | 64 | 53 |
| 0 | 0 | 0 | 0 |
| 28 | 45 | 52 | 30 |
| 6 | 49 | 33 | 31 |
| 9 | 21 | 16 | 0 |
| 13 | 55 | 65 | 24 |

ANOVA results:

Repeated measures ANOVA summary

| Assume sphericity? | No | | | | |
|---|---|---|---|---|---|
| F | 8.575 | | | | |
| P value | 0.0010 | | | | |
| P value summary | *** | | | | |
| Statistically significant (P < 0.05)? | Yes | | | | |
| Geisser-Greenhouse's epsilon | 0.6453 | | | | |
| R squared | 0.3110 | | | | |
| Was the matching effective? | | | | | |
| F | 5.739 | | | | |
| P value | <0.0001 | | | | |
| P value summary | **** | | | | |
| Is there significant matching (P < 0.05)? | Yes | | | | |
| R squared | 0.5686 | | | | |
| ANOVA table | SS | DF | MS | F (DFn, DFd) | P value |
| Treatment (between columns) | 6935 | 3 | 2312 | F (1.936, 36.78) = 8.575 | P = 0.0010 |
| Individual (between rows) | 29394 | 19 | 1547 | F (19, 57) = 5.739 | P < 0.0001 |
| Residual (random) | 15366 | 57 | 269.6 | | |
| Total | 51695 | 79 | | | |
| Data summary | | | | | |
| Number of treatments (columns) | 4 | | | | |
| Number of subjects (rows) | 20 | | | | |
| Number of missing values | 0 | | | | |

### Multiple Comparisons:

| Number of | | | | 1 | | | | |
|---|---|---|---|---|---|---|---|---|
| families | | | | | | | | |
| Number of | | | | 3 | | | | |
| comparisons per | | | | | | | | |
| family | | | | | | | | |
| Alpha | | | | 0.05 | | | | |
| | | 95.00% | | | | | | |
| S̆ídák's multiple | Mean | CI of | Below | | Adjusted | | | |
| comparisons test | Diff. | diff. | threshold? | Summary | P Value | A-? | | |
| JACS Benchmark | −25.10 | −40.32 to | Yes | ** | 0.0011 | B | ML General | |
| vs. ML General | | −9.876 | | | | | condition 1 | |
| condition 1 | | | | | | | | |
| JACS Benchmark | −16.90 | −32.47 to | Yes | * | 0.0310 | C | ML General | |
| vs. ML General | | −1.329 | | | | | condition 2 | |
| condition 2 | | | | | | | | |
| JACS Benchmark | −8.950 | −27.40 to | No | ns | 0.5245 | D | ML General | |
| vs. ML General | | 9.497 | | | | | condition 3 | |
| condition 3 | | | | | | | | |
| | Mean | | Mean | SE of | | | | |
| Test details | 1 | Mean 2 | Diff. | diff. | nl | n2 | t | DF |
| JACS Benchmark | 21.30 | 46.40 | −25.10 | 5.817 | 20 | 20 | 4.315 | 19 |
| vs. ML General | | | | | | | | |
| condition 1 | | | | | | | | |
| JACS Benchmark | 21.30 | 38.20 | −16.90 | 5.950 | 20 | 20 | 2.840 | 19 |
| vs. ML General | | | | | | | | |
| condition 2 | | | | | | | | |
| JACS Benchmark | 21.30 | 30.25 | −8.950 | 7.049 | 20 | 20 | 1.270 | 19 |
| vs. ML General | | | | | | | | |
| condition 3 | | | | | | | | |

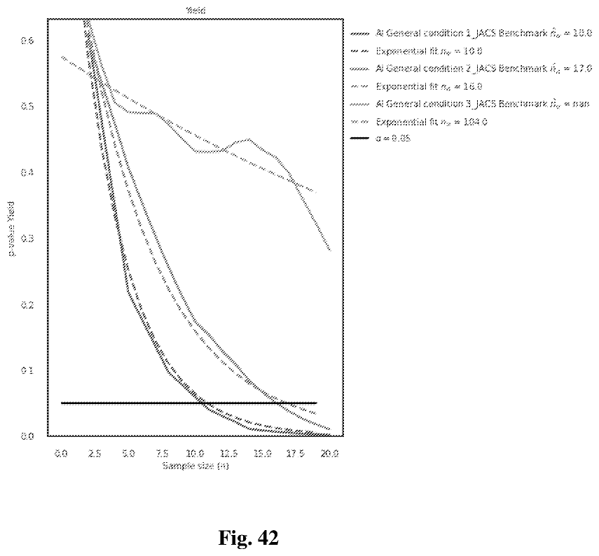

In addition, for FIG. 5B, the p-value of each comparison was calculated as a function of sample size using the pMoSS: p-value Model using the Sample Size method of Gómez-de-Mariscal and coworkers and associated code using standard parameters (FIG. 42). In short, this method models the Mann-Whitney U-test p-value as a sample size dependent function using Monte Carlo cross-validation. In this context, a faster decay of the sample-size-dependent p-value function p (n) to the significance threshold α indicates stronger evidence against the null hypothesis and that statistically significant differences are apparent at smaller sample sizes. From FIG. 42 it can be seen that the statistically significant comparisons in FIG. 5B are conserved (reach the a threshold of 0.05, specifically: general condition 1 vs benchmark, general condition 2 vs benchmark) and differ greatly in relative slope of the exponential decay function compared to the comparison lacking statistical significance (general condition 3 vs benchmark). These differences are apparent at sample sizes as low as 5, and more definitively at 10. These data can also be examined in the context of the binary index θα,γ (from the same reference publication), where θα,γ=1 indicates differences among the datasets being tested and θα,γ=0 indicates non-rejection of the null hypothesis, based on the area-under-the-curve comparisons between the sample size dependent p-value function, a constant function of the significance level α, and the regularization parameter γ. In this context, θα,γ=1 for the comparisons noted as statistically significant in FIG. 4B and θα,γ=0 for the comparison lacking statistical significance. Interestingly, only 10 data points are required to observe statistically significant changes between the top ML discovered general condition and the benchmark condition, whereas the smaller in magnitude statistically significant changes required up to 16 data points. For the comparison that lacked statistical significance, the exponential decay functions approximate >100 data points being required to reach the threshold α=0.05. In this regard, in retrospect these data suggest that the selection of 20 examples in the test set was relatively optimal and that significant effects of sample size dominating p-values are likely not to occur until ˜100 examples.

### Dataset: FIG. 5C

| ML General | ML General | ML General |
|---|---|---|
| condition 1 | condition 2 | condition 3 |
| 6 | 1 | 6 |
| 35 | 16 | 37 |
| 35 | 0 | 3 |
| −21 | −17 | −73 |
| −6 | −35 | −30 |
| 5 | 13 | −4 |
| 26 | 28 | 39 |
| 44 | 14 | 44 |
| −3 | 1 | −12 |
| 28 | 7 | −8 |
| 7 | 4 | −7 |
| 28 | 10 | 12 |
| 80 | 80 | 76 |
| 69 | 54 | 26 |
| 55 | 52 | 41 |
| 0 | 0 | 0 |
| 17 | 24 | 2 |
| 43 | 27 | 25 |
| 12 | 7 | −9 |
| 42 | 52 | 11 |

ANOVA results:

Repeated measures ANOVA summary

| Assume sphericity? | No | | | | |
|---|---|---|---|---|---|
| F | 9.136 | | | | |
| P value | 0.0012 | | | | |
| P value summary | ** | | | | |
| Statistically significant (P < 0.05)? | Yes | | | | |
| Geisser-Greenhouse's epsilon | 0.8450 | | | | |
| R squared | 0.3247 | | | | |
| Was the matching effective? | | | | | |
| F | 14.66 | | | | |
| P value | <0.0001 | | | | |
| P value summary | **** | | | | |
| Is there significant matching (P < 0.05)? | Yes | | | | |
| R squared | 0.8319 | | | | |
| ANOVA table | SS | DF | MS | F (DFn, DFd) | P value |
| Treatment (between columns) | 2608 | 2 | 1304 | F (1.690, 32.11) = 9.136 | P = 0.0012 |
| Individual (between rows) | 39764 | 19 | 2093 | F (19, 38) = 14.66 | P < 0.0001 |
| Residual (random) | 5425 | 38 | 142.8 | | |
| Total | 47797 | 59 | | | |
| Data summary | | | | | |
| Number of treatments (columns) | 3 | | | | |
| Number of subjects (rows) | 20 | | | | |
| Number of missing values | 0 | | | | |

### Multiple Comparisons:

| Number of families | | | 1 | | | | | |
|---|---|---|---|---|---|---|---|---|
| Number of comparisons | | | 2 | | | | | |
| Alpha | | | 0.05 | | | | | |
| | | 95.00% | | | | | | |
| S̆ídák's multiple | Mean | CI of | Below | | Adjusted | | | |
| comparisons test | Diff. | diff. | threshold? | Summary | P Value | A-? | | |
| ML General condition 1 | 8.200 | 0.7859 to | Yes | * | 0.0291 | B | ML | |
| vs. ML General condition | | 15.61 | | | | | General | |
| 2 | | | | | | | condition | |
| | | | | | | | 2 | |
| ML General condition 1 | 16.15 | 7.186 to | Yes | *** | 0.0007 | C | ML | |
| vs. ML General condition | | 25.11 | | | | | General | |
| 3 | | | | | | | condition | |
| | | | | | | | 3 | |
| | Mean | | Mean | SE of | | | | |
| Test details | 1 | Mean 2 | Diff. | diff. | nl | n2 | t | DF |
| ML General condition 1 | 25.10 | 16.90 | 8.200 | 3.054 | 20 | 20 | 2.685 | 19 |
| vs. ML General condition | | | | | | | | |
| 2 | | | | | | | | |
| ML General condition 1 | 25.10 | 8.950 | 16.15 | 3.693 | 20 | 20 | 4.373 | 19 |
| vs. ML General condition | | | | | | | | |
| 3 | | | | | | | | |

### Dataset: FIG. 5E

| | JACS Benchmark | ML Condition 1 |
|---|---|---|
| 12 | 0.067353437 | 0.027555 |
| 13 | 0 | 0 |
| 14 | 0.015029023 | 0.05466772 |
| 15 | 0.053068651 | 0 |
| 16 | 0 | 0 |
| 17 | 0 | 0 |
| 18 | 0.087576689 | 0.05777277 |
| 19 | 0.064482514 | 0.08657081 |
| 20 | 0 | 0 |
| 21* | | |
| 22 | 0.067242793 | 0.03219622 |
| 23 | 0.033719868 | 0.03889935 |
| 24 | 0.062817709 | 0.06145812 |
| 25 | 0 | 0 |
| 26 | 0 | 0 |
| 27 | 0.075890737 | 0.05265572 |
| 28 | 0.216379087 | 0.11209111 |
| 29 | 0.177395206 | 0.11610443 |
| 30 | 0 | 0 |

*For reaction 21, protodeboronation could not be distinguished from the internal standard

### Paired t Test Results:

| Column B | ML Condition 1 |
|---|---|
| vs. | vs. |
| Column A | JACS Benchmark |
| Paired t test | |
| P value | 0.0665 |
| P value summary | ns |
| Significantly different (P < 0.05)? | No |
| One- or two-tailed P value? | Two-tailed |
| t, df | t = 1.961, df = 17 |
| Number of pairs | 18 |
| How big is the difference? | |
| Mean of differences (B − A) | −0.01561 |
| SD of differences | 0.03377 |
| SEM of differences | 0.007960 |
| 95% confidence interval | −0.03241 to 0.001185 |
| R squared (partial eta squared) | 0.1845 |
| How effective was the pairing? | |
| Correlation coefficient (r) | 0.8717 |
| P value (one tailed) | <0.0001 |
| P value summary | **** |
| Was the pairing significantly effective? | Yes |

### Dataset: FIG. 5F

| | JACS Benchmark | ML Condition 1 |
|---|---|---|
| 12 | 0.158613 | 0.060332 |
| 13 | 0.064829 | 0 |
| 14 | 0.443649 | 0 |
| 15 | 0.340244 | 0 |
| 16 | 0.048406 | 0 |
| 17 | 0.288492 | 0 |
| 18 | 0.432289 | 0.232916 |
| 19 | 0.018501 | 0 |
| 20 | 0.151719 | 0.069811 |
| 21 | 0.053956 | 0.64168 |
| 22 | 0.135498 | 0.102325 |
| 23 | 0.010973 | 0 |
| 24 | 0.133059 | 0.141509 |
| 25 | 0.418732 | 0.408757 |
| 26 | 0.521864 | 0.273056 |
| 27 | 0.623776 | 0.295024 |
| 28 | 0.14049 | 0.013437 |
| 29 | 0.244228 | 0.168692 |
| 30 | 0.200142 | 0.010607 |

### Paired t Test Results:

| Column B | ML Condition 1 |
|---|---|
| vs. | vs. |
| Column A | JACS Benchmark |
| Paired t test | |
| P value | 0.0444 |
| P value summary | * |
| Significantly different (P < 0.05)? | Yes |
| One- or two-tailed P value? | Two-tailed |
| t, df | t = 2.161, df = 18 |
| Number of pairs | 19 |
| How big is the difference? | |
| Mean of differences (B − A) | −0.1059 |
| SD of differences | 0.2135 |
| SEM of differences | 0.04898 |
| 95% confidence interval | −0.2088 to −0.002955 |
| R squared (partial eta squared) | 0.2060 |
| How effective was the pairing? | |
| Correlation coefficient (r) | 0.2899 |
| P value (one tailed) | 0.1143 |
| P value summary | ns |
| Was the pairing significantly effective? | No |

### Dataset: FIG. 5G

| | JACS Benchmark | ML Condition 1 |
|---|---|---|
| 12 | 0.07 | 0.25 |
| 13 | 0.03 | 0.61 |
| 14 | 0.06 | 1.12 |
| 15 | 0.23 | 1 |
| 16 | 0 | 0.29 |
| 17 | 0.41 | 0.78 |
| 18 | 0.11 | 0.16 |
| 19 | 0.86 | 0.83 |
| 20 | 0.12 | 0.28 |
| 21 | 2.14 | 2.34 |
| 22 | 0.03 | 0.18 |
| 23 | 0.74 | 0.73 |
| 24 | 0.07 | 0.13 |
| 25 | 0.2 | 0.12 |
| 26 | 0.1 | 0.55 |
| 27 | 0.05 | 0.44 |
| 28 | 0.04 | 0.13 |
| 29 | 0.31 | 0.55 |
| 30 | 0.07 | 0.45 |

### Paired t Test Results:

| Column B | ML Condition 1 |
|---|---|
| vs. | vs. |
| Column A | JACS Benchmark |
| Paired t test | |
| P value | 0.0005 |
| P value summary | *** |
| Significantly different (P < 0.05)? | Yes |
| One- or two-tailed P value? | Two-tailed |
| t, df | t = 4.220, df = 18 |
| Number of pairs | 19 |
| How big is the difference? | |
| Mean of differences (B − A) | 0.2789 |
| SD of differences | 0.2881 |
| SEM of differences | 0.06610 |
| 95% confidence interval | 0.1401 to 0.4178 |
| R squared (partial eta squared) | 0.4973 |
| How effective was the pairing? | |
| Correlation coefficient (r) | 0.8445 |
| P value (one tailed) | <0.0001 |
| P value summary | **** |
| Was the pairing significantly effective? | Yes |

### Dataset: FIG. 41

| | JACS Benchmark | ML Condition 1 |
|---|---|---|
| 12 | 0 | 0.01633437 |
| 13 | 0 | 0 |
| 14 | 0 | 0.01450601 |
| 15 | 0 | 0 |
| 16 | 0 | 0 |
| 17 | 0 | 0 |
| 18 | 0 | 0 |
| 19 | 0.064482514 | 0.08657081 |
| 20 | 0 | 0.01045528 |
| 21 | 0 | 0 |
| 22 | 0 | 0 |
| 23 | 0 | 0.04360252 |
| 24 | 0 | 0 |
| 25 | 0 | 0.02380896 |
| 26 | 0.521864283 | 0.13450683 |
| 27 | 0.075890737 | 0.05265572 |
| 28 | 0.012715364 | 0.13684107 |
| 29 | 0 | 0 |
| 30 | 0.014545898 | 0.01921547 |

### Paired t Test Results:

| Column B | ML Condition 1 |
|---|---|
| vs. | vs. |
| Column A | JACS Benchmark |
| Paired t test | |
| P value | 0.7244 |
| P value summary | ns |
| Significantly different (P < 0.05)? | No |
| One- or two-tailed P value? | Two-tailed |
| t, df | t = 0.3582, df = 18 |
| Number of pairs | 19 |
| How big is the difference? | |
| Mean of differences (B − A) | −0.007947 |
| SD of differences | 0.09671 |
| SEM of differences | 0.02219 |
| 95% confidence interval | −0.05456 to 0.03867 |
| R squared (partial eta squared) | 0.007078 |
| How effective was the pairing? | |
| Correlation coefficient (r) | 0.6533 |
| P value (one tailed) | 0.0012 |
| P value summary | ** |
| Was the pairing significantly effective? | Yes |

### INCORPORATION BY REFERENCE

All US patents and US and PCT patent application publications mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A method, comprising:
selecting a reaction pair comprising a first molecule and a second molecule;
wherein the first molecule is selected from a first matrix and the second molecule is selected from a second matrix;
selecting one or more reaction conditions for the reaction pair, the selection based on historic use of the one or more reaction conditions and a structural and functional diversity of the selected reaction pair;
automatically performing, by a robotic system, an initial round of reactions between the selected reaction pair under the selected one or more reaction conditions;
optimizing, using a machine learning model, the one or more reaction conditions associated with the initial round of reactions based on a yield of each reaction in the initial round of reactions;
determining an optimized series of reactions to be performed;
performing a reaction of the optimized series of reactions, thereby forming a product, and predicting yields thereof; and
outputting an optimized set of general reaction conditions for each optimized reaction between the selected reaction pair.

2. The method of claim 1, further comprising minimizing uncertainty in the machine learning model.

3. The method of claim 2, wherein minimizing uncertainty comprises:
constructing a surrogate model to predict one or more reaction yields from the reaction pair;
estimating an objective function of a performed reaction based on a predicted output from the surrogate model; and
estimating the objective function for an unperformed reaction.

4. The method of any one of claims 1-3, wherein selecting a reaction pair comprises:
clustering the first matrix by a common ring structure and pendant functionality, thereby generating a first centroid; and wherein the first centroid comprises the closest representatives of the first matrix;
selecting the second molecule from the second matrix;
identifying all combinations comprising the first centroid and the second molecule, thereby generating a chemical space; and
comparing the chemical space to a corpus of chemical products, thereby generating a product space.

5. The method of claim 4, further comprising:
applying a greedy algorithm to the product space; and
identifying a set of first centroid and second molecule pairs to maximize mutual dissimilarity of a resulting product of the reaction pair.

6. The method of any one of claims 1-5, wherein selecting the one or more reaction conditions further comprises:
considering at least one of a solvent, a base, a catalyst, and a temperature as a variable for the one or more reaction conditions; and
determining, based on analysis of prior use of the one or more reaction conditions, a set of initial conditions.

7. The method of claim 6, wherein the solvent is 5:1 dioxane: water.

8. The method of claim 6 or 7, wherein the base is K₃PO₄ or Na₂CO₃.

9. The method of any one of claims 6-8, wherein the catalyst is a palladium catalyst, and optionally further comprises a ligand.

10. The method of claim 9, wherein the ligand is selected from the group consisting of SPhos, XPhos, and triphenylphosphine (PPh₃).

11. The method of any one of claims 6-10, wherein the catalyst is selected from the group consisting of Pd(SPhos) G4, Pd(PPh₃)₄ and Pd(XPhos) G4.

12. The method of any one of claims 6-11, wherein the temperature is about 50° C. to about 150° C.

13. The method of any one of claims 6-12, wherein the temperature is about 60° C. or about 100° C.

14. The method of any one of claims 1-13, wherein optimizing the one or more reaction conditions comprises:
performing a reaction under the one or more reaction conditions, thereby forming a product; and
determining parameters for the one or more reaction conditions outputting the highest yield.

15. The method of any one of claims 1-13, wherein optimizing the one or more reaction conditions comprises:
identifying one or more catalysts with similar yields for different substrates; and
removing the identified one or more catalysts from possible reaction conditions, thereby decreasing redundancy.

16. The method of any one of claims 1-15, further comprising:
iteratively repeating the optimizing of the one or more reaction conditions, thereby forming a product;
measuring a yield of the product; and
determining a threshold yield is met.

17. The method of any one of claims 1-15, further comprising:
generating, by the machine learning model, small datasets for optimization, wherein the small datasets include negative data.

18. The method of any one of claims 1-17, wherein the first molecule comprises a halo-substituted aryl or heteroaryl.

19. The method of any one of claims 1-18, wherein the first molecule comprises a compound of formula (Ia): 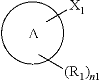
wherein:
A is aryl or heteroaryl;
each R₁ is independently selected from the group consisting of alkyl, alkoxyl, alkenyl, alkynyl, aralkyl, heteroaryl(alkyl), aryl, heteroaryl, halo, haloalkyl, hydroxyl, carboxyl, acyl, ester, amino, amido, cyano, cycloalkyl, and heterocycloalkyl;
n1 is 0, 1, 2, 3, 4, or 5; and
X₁ is halo.

20. The method of claim 19, wherein X₁ is bromo.

21. The method of any one of claims 18-20, wherein the first molecule is selected from the group consisting of:

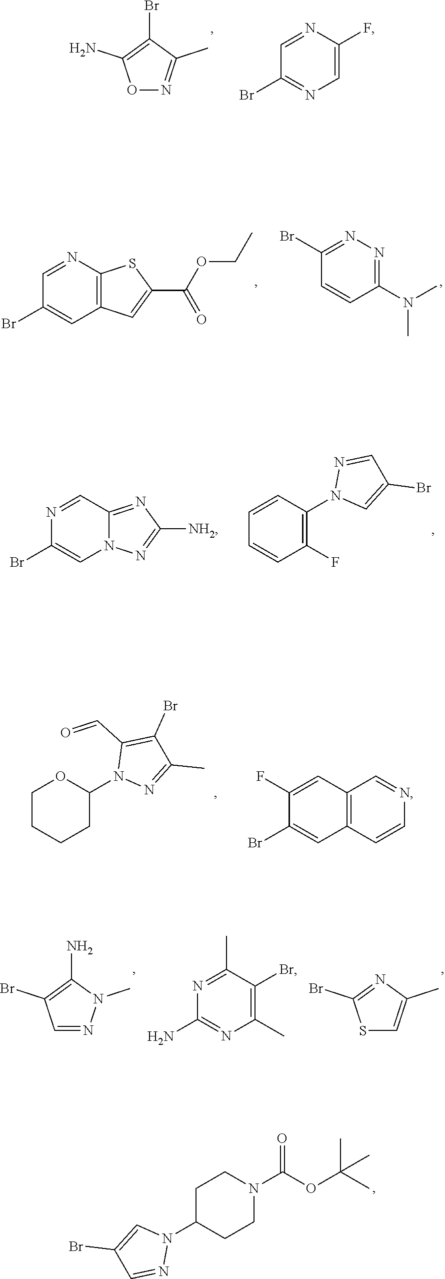

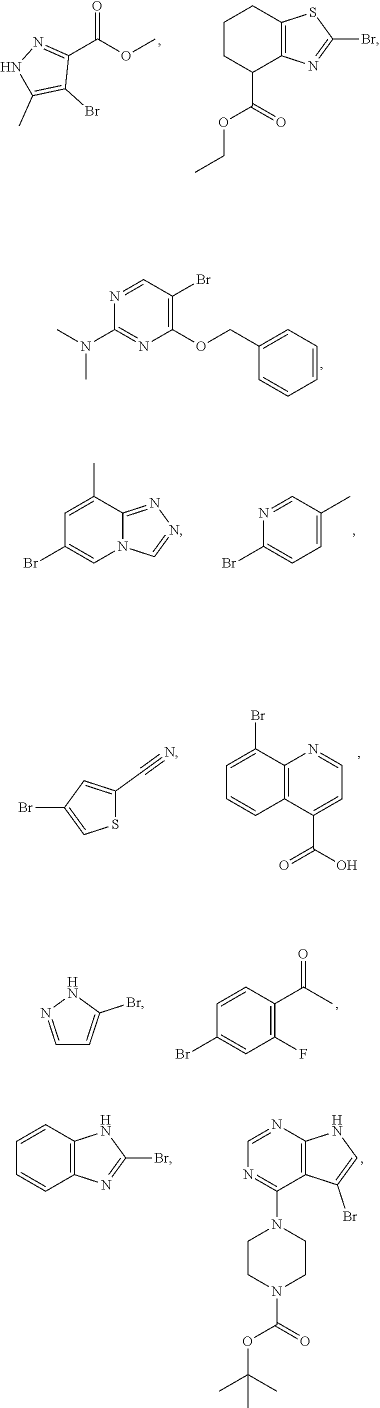

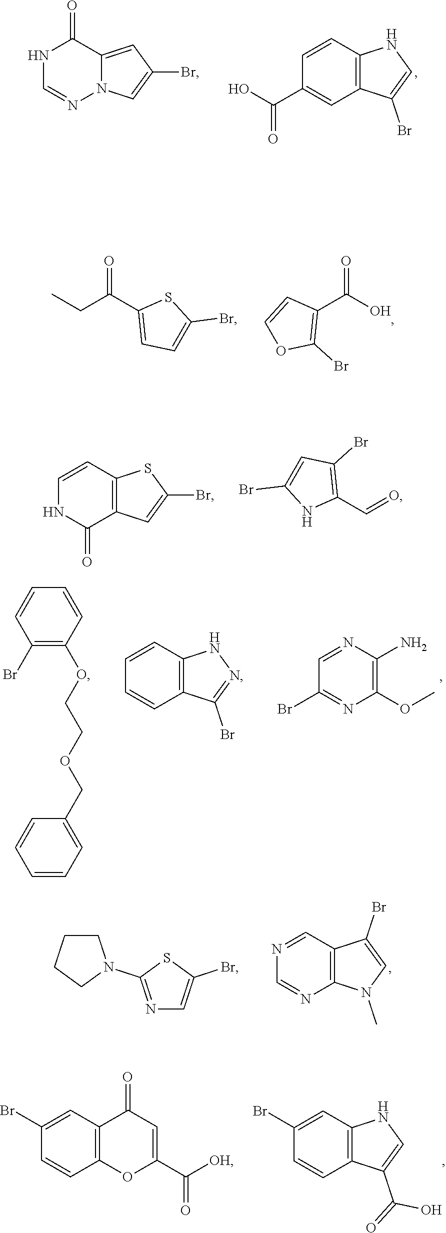

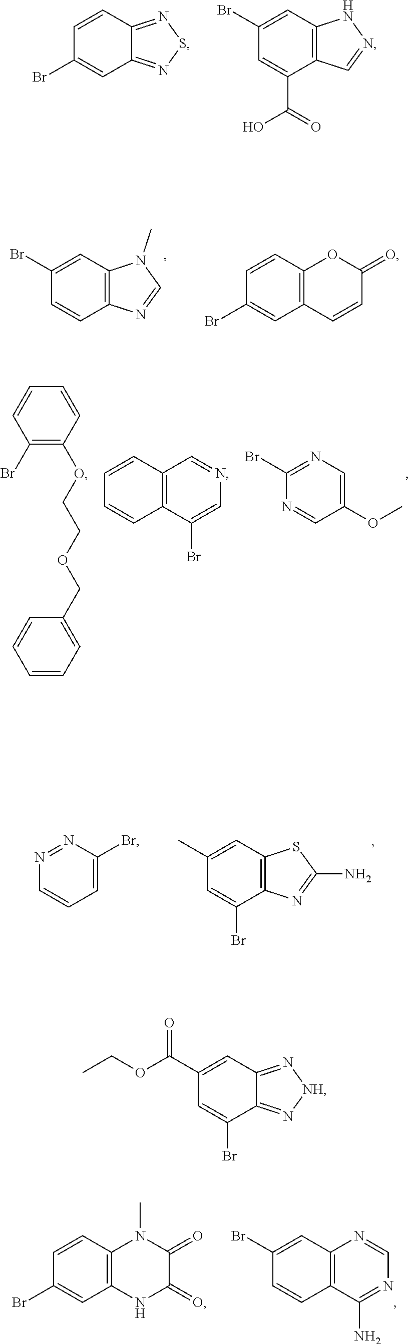

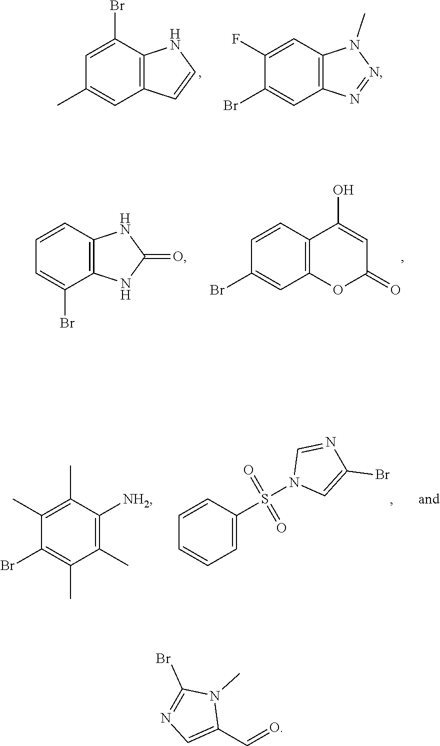

22. The method of any one of claims 1-21, wherein the second molecule comprises an aryl or heteroaryl further comprising a boronic acid, a boronic acid ester, or a tetrafluoroborate salt.

23. The method of any one of claims 1-22, wherein the second molecule comprises a compound of formula (Ib): 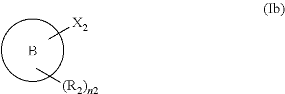
wherein:
B is aryl or heteroaryl;
each R₂ is independently selected from the group consisting of alkyl, alkoxyl, alkenyl, alkynyl, aralkyl, heteroaryl(alkyl), aryl, heteroaryl, halo, haloalkyl, hydroxyl, carboxyl, acyl, ester, amino, amido, cyano, cycloalkyl, and heterocycloalkyl;
n2 is 0, 1, 2, 3, 4, or 5; and
X₂ is selected from the group consisting of N-methylimidodiacetic acid boronic acid ester, tetramethyl N-methyliminodiacetic acid boronic acid ester, pinacol boronic acid ester, boronic acid, or a tetrafluoroborate salt.

24. The method of claim 22 or 23, wherein the second molecule is selected from the group consisting of: 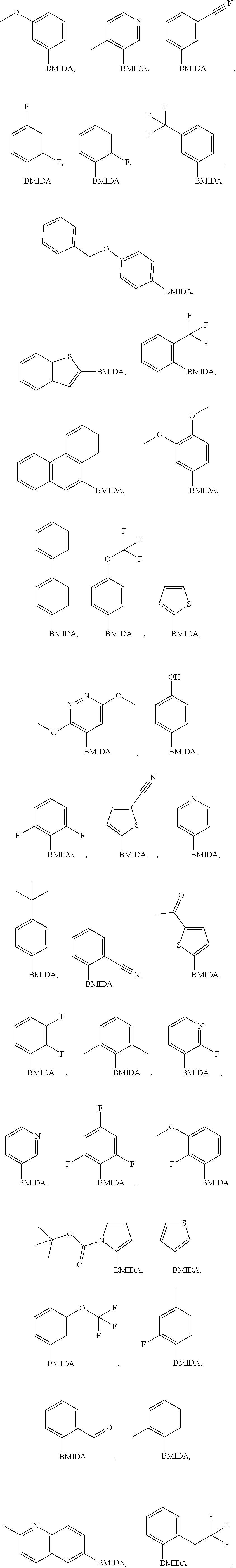 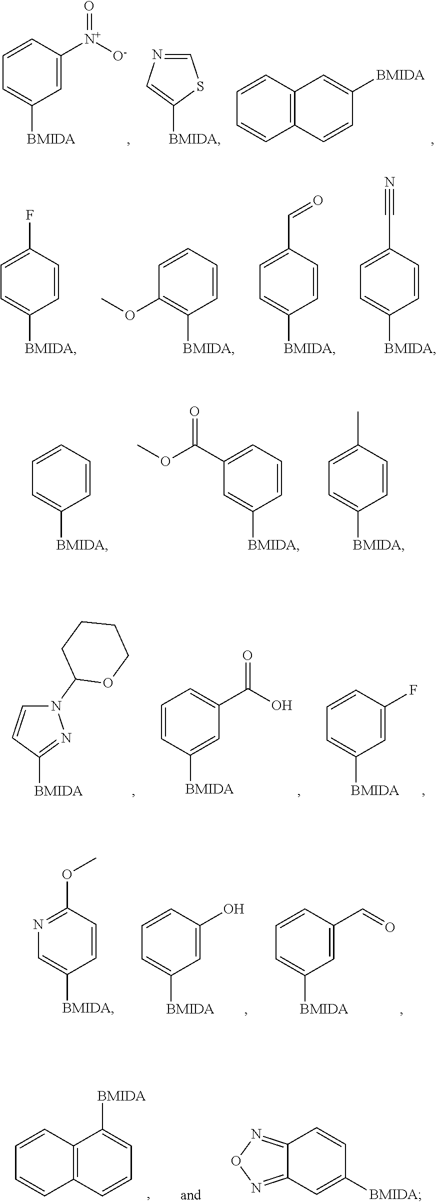
wherein BMIDA is N-methylimidodiacetic acid boronic acid ester.

25. A system, comprising:
a robotic system; and
a computing node comprising a computer readable storage medium having program instructions embodied therewith, the program instructions executable by a processor of the computing node to cause the processor to perform a method comprising the method of any one of claims 1-24.
